(19) United States
(12) Patent Application Publication
(10) Pub. No.: US 2013/0157932 A1
(43) Pub. Date: Jun. 20, 2013

(54) NGAL AND URINARY TRACT INFECTION

(71) Applicants: The Trustees of Columbia University in the City of New York, New York, NY (US); Jonathan Barasch, New York, NY (US); Neal Paragas, New York, NY (US)

(72) Inventors: Jonathan BARASCH, New York, NY (US); Neal Paragas, New York, NY (US)

(73) Assignee: The Trustees of Columbia University in the City of New York, New York, NY (US)

(21) Appl. No.: 13/671,533

(22) Filed: Nov. 7, 2012

Related U.S. Application Data
(63) Continuation-in-part of application No. PCT/US11/35757, filed on May 9, 2011.
(60) Provisional application No. 61/332,477, filed on May 7, 2010.
(60) Provisional application No. 61/347,954, filed on May 25, 2010.

(51) Int. Cl. A61K 38/17; A61K 31/7028; A61K 31/739

(52) U.S. Cl. CPC A61K 38/1709; A61K 31/739; A61K 31/7028
USPC 514/2.8; 514/54; 514/25; 435/29; 435/6.13; 435/6.11; 435/7.1; 435/6.12

(57) ABSTRACT

In some embodiments, the present invention is directed to compositions and methods for the treatment and prevention of urinary tract infections (UTIs) and urosepsis. In some embodiments, the methods of the invention comprise administering to a subject in need thereof a therapeutically effective amount of an agent that stimulates genito-urinary tract epithelial cells to produce NGAL, and/or an NGAL protein or a functional derivative thereof, and optionally also administering to the subject an additional agent useful for treating or preventing UTI or urosepsis. In other embodiments, the present invention also provides methods of screening for agents that stimulate urinary tract epithelial cells to produce NGAL.

This application is a continuation-in-part of International Application No. PCT/US2011/035757, filed on May 9, 2011, which claims the benefit of U.S. Provisional Patent Application No. 61/332,477, filed May 7, 2010, and U.S. Provisional Patent Application No. 61/347,954, filed May 25, 2010, the contents of each of which are hereby incorporated by reference.

For the purposes only of the U.S. and other jurisdictions that permit incorporation by reference, all patents, patent applications and publications cited herein are hereby incorporated by reference in their entireties.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Urinary tract infections or “UTIs” are one of the most prevalent and resource taxing diseases in the U.S., with 13.3% (12.8 million) of all women and 2.3% (2 million) of all men in the U.S. infected annually, resulting in an annual cost to the U.S. healthcare system of around $3.5 billion. In 2000 there were an estimated 11.02 million visits (2.05 million men; 8.97 million women) to a physician's office or hospital related to UTI. Uropathogenic Eschereicia coli or “UPEC” bacteria are involved in 70-95% of all cases of UTI. Many of these UPEC bacteria rely on catecholate-siderophores as their primary iron uptake mechanism.

Neutrophil Gelatinase Associated Lipocalin (NGAL) is a small secreted protein with a molecular weight of about 22 kD, and is a siderophore and iron binding protein. A siderophore is an organic molecule that binds to and chelates iron. Bacteria produce siderophores such as enterochelin. Mammals endogenously produce a siderophore called catechol. Enterochelin has an extremely high affinity for iron, and NGAL has a high affinity for the enterochelin-iron complex.

### SUMMARY OF THE INVENTION

The present invention is based, in part, on certain discoveries which are described more fully in the Examples section of the present application. For example, the present invention is based, in part, on the discovery that in response to infection of the urinary tract with enterochelin-dependent uropathogenic bacteria, epithelial cells of the genitourinary tract secrete NGAL protein, which has bacteriostatic activity and inhibits growth of bacteria. The present invention is also based, in part, on the elucidation of the biochemical pathways that result in the secretion of NGAL protein by epithelial cells of the genitourinary tract in response to a urinary tract infection.

In one embodiment, the present invention provides a method for treating or preventing infection of the urinary tract with a bacterium, such as an enterochelin-dependent uropathogenic bacterium, in a subject, or treating or treating or preventing urosepsis resulting from such an infection, the method comprising administering to the subject a therapeutically effective amount of one or more agents selected from the group consisting of: (a) an agent that stimulates genito-urinary tract epithelial cells of the subject to secrete NGAL protein, (b) NGAL, and (c) a functional derivative thereof, or combinations of one or more thereof. In some embodiments, the bacterium is an enterochelin-dependent bacterium, such as an enterochelin-dependent uropathogenic E. coli or “UPEC” bacterium. In some embodiments the agent that stimulates genito-urinary tract epithelial cells to secrete NGAL protein stimulates the secretion of NGAL by epithelial cells of the kidney, such as epithelial cells of the collecting duct or epithelial cells of the thick ascending limb of Henlé in the kidney. In some embodiments, the agent that stimulates genito-urinary tract epithelial cells of the subject to secrete NGAL protein is an NFκB activator, an activator of a TLR-NFκB pathway (such as an activator of a TLR4-NFκB or TLR11-NFκB pathway), a NRF2 modulator, a HIF modulator, or a non-toxic derivative of either lipid A, lipopolysaccharide, or endotoxin. In some embodiments the agents are administered systemically. In other embodiments the agents are administered locally.

In another embodiment, the present invention provides a method for treating or preventing infection of the urinary tract with a bacterium, such as an enterochelin-dependent uropathogenic bacterium, in a subject, or treating or preventing urosepsis resulting from such an infection, the method comprising stimulating genito-urinary tract epithelial cells of the subject to secrete NGAL protein. In some embodiments, the bacterium is an enterochelin-dependent uropathogenic bacterium, such as an enterochelin-dependent uropathogenic E. coli (UPEC) bacterium. In some embodiments, the genito-urinary tract epithelial cells are kidney epithelial cells, such as epithelial cells of the collecting duct, or epithelial cells of the thick ascending limb of Henlé. In some embodiments the step of stimulating genito-urinary tract epithelial cells to secrete NGAL protein comprises administering to the subject a therapeutically effective amount one or more agent selected from the group consisting of: (a) a non-toxic derivative of lipid A, (b) a non-toxic derivative of lipopolysaccharide, (c) a non-toxic derivative of endotoxin, (d) an activator of the TLR4-NFkB pathway, (e) an activator of the TLR11-NFkB pathway, (f) an NFκB activator, (g) a NRF2 modulator, and (h) a HIF modulator, or a combination of one or more thereof. In some embodiments such agents are administered systemically to the subject. In other embodiments such agents are administered locally to the genitourinary tract of the subject.

In other embodiments, the present invention provides pharmaceutical compositions for use in treating a urinary tract infection or urosepsis, the compositions comprising a therapeutically effective amount of an agent that stimulates genito-urinary tract epithelial cells to secrete NGAL protein, and optionally a therapeutically effective amount of NGAL, or a functional derivative thereof. In some embodiments the agent that stimulates genito-urinary tract epithelial cells to secrete NGAL protein is selected from the group consisting of: (a) a non-toxic derivative of lipid A, (b) a non-toxic derivative of lipopolysaccharide, (c) a non-toxic derivative of endotoxin, (d) an activator of the TLR4-NFkB pathway, (e) an activator of the TLR11-NFkB pathway, (f) an NFκB activator, (g) a NRF2 modulator, and (h) a HIF modulator.

In yet other embodiments, the present invention provides methods of screening for agents that stimulate epithelial cells of the urinary tract, such as kidney epithelia cells (including epithelial cells of the collecting ducts or of the thick ascending limb of Henlé), bladder epithelial cells, and urethral epithelial cells, to produce NGAL mRNA or protein. In some embodiments, such screening methods comprise providing a population of urinary tract epithelial cells, contacting the population of urinary tract epithelial cells with one or more test agents, and testing for production of NGAL mRNA or protein by the urinary tract epithelial cells, thereby identifying agents that stimulate production of NGAL mRNA or protein by the urinary tract epithelial cells. In some such embodiments, the urinary tract epithelial cells may be in vivo, for example in a mouse model. In other embodiments, the urinary tract epithelial cells may be cultured in vitro. Urinary tract epithelial cells that are cultured in vitro may be primary cultures, or may be derived from primary cultures, or may be cell lines, such as established urinary tract epithelial cell lines, including kidney epithelial cell lines, bladder epithelial cells lines, urethral epithelial cell lines, and the like. The test agents may be any suitable test agents, including, but not limited to, libraries of small molecule drugs, libraries of proteinaceous or peptide drugs (including peptidomimetic drugs), libraries of antibodies, libraries of RNA molecules (including, but not limited to, antisense RNAs, siRNAs, shRNAs, and microRNAs, ribozymes), and the like. In addition to libraries of test agents, individual test agents, or smaller populations of test agents, may also be used. Any suitable means may be used to detect NGAL production by the urinary tract epithelial cells. In one embodiment, secreted NGAL protein is detected in cell supernatants. In another embodiment, NGAL protein within the epithelial cells is detected. NGAL protein may be detected using any suitable means. In one embedment, NGAL protein is detected using an antibody to NGAL. The NGAL antibody may be labeled with a detectable moiety, or a secondary antibody that is labeled with a detectable moiety may be used. Suitable detectable moieties may include enzyme subtstrates (such as horseradish peroxidase, alkaline phosphatase, and the like), and fluorescent labels (such as green fluorescent protein, and the like). In one embodiment NGAL protein may be detected in an ELISA assay using an anti-NGAL antibody. In another embodiment NGAL mRNA is detected. NGAL mRNA may be detected using any suitable means, including, but not limited to, in situ hybridization, Northern blotting, PCR, QPCR, and the like. Any suitable probes or primers for detection of NGAL mRNA may be used.

In one embodiment, the present invention provides a method for treating or preventing infection of the urinary tract with a bacterium, such as an enterochelin-dependent uropathogenic bacterium, in a subject. In another embodiment, the present invention is directed to treating or preventing pyelonephritis and cystitis resulting from such an infection.

The methods comprises administering to the subject a therapeutically effective amount of one or more agents selected from the group consisting of: (a) an agent that stimulates α-intercalated cells (α-ICs) of the subject to secrete Neutrophil Gelatinase Associated Lipocalin (NGAL)-Siderocalin (Scn) protein, (b) NGAL-Scn, and (c) a functional derivative thereof, or combinations of one or more thereof.

In some embodiments, the bacterium is an enterochelin-dependent bacterium, such as an enterochelin-dependent uropathogenic E. coli or “UPEC” bacterium. In some embodiments the agent that stimulates α-ICs to secrete NGAL-Scn protein stimulates the secretion of NGAL-Scn. In some embodiments the agents are administered systemically. In other embodiments the agents are administered locally.

In other embodiments, the present invention provides pharmaceutical compositions for use in treating a urinary tract infection or urosepsis, the compositions comprising a therapeutically effective amount of an agent that stimulates genito-urinary tract epithelial cells to secrete NGAL-Scn protein, and optionally a therapeutically effective amount of NGAL-Scn, or a functional derivative thereof.

These and other embodiments of the invention are further described in the following sections of the application, including the Detailed Description, Examples, Claims, and Drawings.

### BRIEF DESCRIPTION OF THE FIGURES

The patent or application file contains at least one drawing executed in color. Copies of this patent or patent application publication with color drawing(s) will be provided by the United States Patent and Trademark Office upon request and payment of the necessary fee.

FIG. 1: The kidney is an exocrine organ which defends the urinary system from infection by secreting NGAL. NGAL has been shown to be a binding protein for gram-negative siderophores such as enterochelin. To establish whether uNGAL can inhibit the growth of a highly pathogenic uropathogenic Escherichia coli strain (CFT073) NGAL activity was investigated both in vitro and in vivo. (a) An in vitro culture of CFT073 UPEC in minimal media (M9), M9 containing iron (1 mM FeC13), M9 and uNGAL (5 μM), and M9 with uNGAL and high iron, showed that the presence of uNGAL can inhibit growth of the UPEC and that inhibition could be reversed by addition of high iron. Bacterial growth was assessed by measuring the optical density at indicated time points. Results from each treatment were compared with the results from Medium alone using unpaired T test. (b) To determine the in vivo role of uNGAL, sibling matched wild-type animals and Ngal globally deleted animals (EIIa-cre) were challenged with a transurethral (TU) injection of CFT073. CFT073 presence in the GU correlated to uNGAL as seen in the Western blot (WB). Ngal deleted animals compared to wild-type (WT) took significantly longer to clear a CFT073 infection—6 days compared to 3 days for a wild type.

FIG. 2: Generation of the NgalloxP/loxP conditional knockout murine model. (a) A loxP site was inserted into intron 1 (light gray arrowhead), a neomycin cassette (light blue box) into intron 5 flanked by FRT (dark gray arrowhead) and loxP sites on the 5′ and 3′ end. Neomycin cassette was excised by breeding the heterozygous NgalloxPneo/+ with a Flippase Recombinase (FLP) mouse to make the Ngalloxp/+. Ngalloxp/+ was bred against C57BL/6 animals to remove the FLP gene. Ngalloxp/+ were subsequently bred against EIIa-Cre mice for site-specific recombination between the intron 1 loxP site and the intron 5 loxP site creating a NgalloxP/+, EIIa-Cre mouse. (b) Genotyping strategy of the Ngal loxP-cre system.

FIG. 3: The distal nephron and the bladder epithelium responds to an uropathogenic infection by secreting uNGAL. (a, b) In situ hybridization demonstrated Ngal mRNA expression in the bladder epithelium (a) and in collecting ducts (CD) of the kidney (b) 24 hours after an acute UTI (10-20 μl of 5×10⁹ CFU/ml CFT073). Ngal mRNA was predominantly expressed in a few CD and was absent all other epithelial tubules and the thin limb of Henle. (c) In order to determine the contribution of uNGAL from the kidney and the bladder, QPCR was done 1 day post-TU and Ngal copy number was determined. Almost 3 log orders greater Ngal copies increase were observed in the kidney compared to the bladder.

FIG. 4: Toll dependence of NGAL expression in Kidney. (a) LPS-insensitive mice, C3H/HeJ have significantly less uNGAL than wild type animals after i.p. challenge with LPS. To determine the origin of uNGAL reciprocal cross-transplants of C3H/HeJ kidneys to C3H/HeOuJ bodies were performed, and vice versa. It was found that C3H/HeJ kidneys produce significantly less Ngal than C3H/HeOuJ animals (p<0.0005). (b) In situ hybridization of these kidneys revealed a standard pattern of LPS induced Ngal expression in the distal epithelia of the kidney in the wild type kidney compared to the knockout kidney while Ngal mRNA was absent in the LPS-insensitive animal. These data confirmed that kidney epithelia derived Ngal is expressed in the TLR:NFkB pathway. (c) Investigation of the receptors involved in the UPEC activation of Ngal expression. C57BL/6, Tlr2, Tlr4, and Tlr11 knockout animals were challenged with an acute UTI (10-20 ul of 5×109 CFU/ml CFT073). Tlr11 showed a similar phenotype to the Ngal KO.

FIG. 5: Box plots of urinary neutrophil gelatinase-associated lipocalin (uNGAL) in patients with a UTI in multicenter study. (a) Patients presenting with gram negative UTIs had abundant uNGAL compared to patients with gram positive infections (2078±3215 vs 592±1242 mg/g creatinine, p=0.01). One patient in the gram negative group had an uNGAL of 16,891 mg/g creatinine, not shown. (b) uNGAL parallel presence of urinary colony forming units (cfu). Patients with >100,000 cfu had significantly more uNGAL compared to patients with 10,000 and 100,000 cfu (2251±3353 mg/g creatinine, 928±1850 mg/g creatinine, p=0.02). Only one patient had <10,000 cfu of bacteria in their urine, and had an uNGAL level of 88 mg/g creatinine One patient with more than 100,000 cfu had an uNGAL of 16,891 mg/g creatinine, not shown. (c) uNGAL concentration trended to increase with rising number of white blood cells. Patients with 11-20 and >30 cells per high powered field (hpf) had significantly more uNGAL compared to patients with 3-5 cells per hpf (1455±1543 mg/g creatinine, 3023±4067 mg/g creatinine, vs 219±309 mg/g creatinine, p=0.002 and <0.001, respectively). uNGAL levels were not significantly different between any other groups. uNGAL in patients with between 6 and 10 cells per hpf and those with between 21 and 30 cells hpf are 1365±2531 mg/g creatinine and 1342±2422 mg/g creatinine One patient in the >30 cells group had an uNGAL of 16,891 mg/g creatinine, not shown.

FIG. 6: NGAL is synthesized by bladder and kidney in a UTI. The distal nephron and the bladder epithelium responds to an uropathogenic infection by secreting uNGAL. (a, b) In situ hybridization demonstrated NGAL mRNA expression in the bladder epithelium (b) and in collecting ducts of the kidney (c) 1 d after an acute UTI (10-20 μl of 5×10⁹ CFU/ml CFT073). NGAL mRNA was predominantly expressed in the CD and was absent all other epithelial. (c) In order to determine the contribution of uNGAL from the kidney and the bladder, QPCR was done 1 d post-TU and Ngal copy number was determined. Almost 3 log orders greater Ngal copy number increase was seen in the kidney (left-hand column) compared to the bladder (right-hand column). (d) Primary culture of Ngal-Luc2/mC kidney medulary tissue and bladder tissue showing the responsiveness of uNGAL to a bacterial infection.

FIG. 7: The kidney is the dominant source of urinary NGAL driven by TLR activation. Toll dependence of NGAL expression in Kidney. Previously it was observed that LPS-insensitive mice, C3HeJ, had significantly less uNGAL than wild type animals after i.p. challenge with LPS. To determine the origin of uNGAL reciprocal cross-transplants were performed of C3H/HeJ kidneys to C3H/HeOuJ bodies, and vice versa. It was found that C3H/HeJ kidneys produce significantly less Ngal than C3H/HeOuJ animals (p<0.0005) (a). These data confirmed that Ngal is expressed in the TLR:NFkB pathway. (b) To further investigate the receptors involved in the UPEC activation of Ngal expression during a UTI, C3HHeN control and C3HHeJ TLR4 mutant animals were challenged with an acute UTI (10-20 μl of 5×10⁹ CFU/ml CFT073). C3H/HeN animals had a lower urinary bacterial burden compared to C3H/HeJ mice. (c) We further found that C3H/HeJ kidney Ngal expression was significantly reduced (˜20 fold) compared to C3H/HeN wild type animals. These results indicate that TLR4:NFkB pathway senses a UTI and activates uNGAL expression. (d) We generated a Ngal conditional KO in order to localize NGAL expressing cells during a UTI. By knocking out Ngal expression in the collecting ducts by breeding the HoxB7-cre recombinase animal with the NgalloxP/loxP animal, it was found that 1 day post-TU these animals had an intermediate phenotype to the global KO (EIIaCre) and the wild type in median CFU/ml urine.

FIGS. 8A-I. Kidney NGAL-Scn inhibited growth of uropathogenic bacteria in a model of cystitis. FIG. 8A. To model human cystitis, we monitored uNGAL-Scn and uCFUs longitudinally for 7 d after we challenged sibling matched wild-type (n=7) and Ngal-Scn^{-/-} (n=6) with a UPEC strain, CFT073 (UPEC) (20 μl of 5×10⁸ CFU ml⁻¹, i.e. approximately 1×10⁷ CFU) by TU inoculation. Significantly delayed UPEC clearance (knockout: 6 d; wild type: 3 d) was found in Ngal-Scn^{-/-} mice. FIG. 8B. Longitudinal urine samples taken over 7 d from both WT and Ngal-Scn^{-/-}. uNGAL-Scn (demonstrated by western blot) correlated with UPEC uCFU. FIG. 8C. UPEC in urine (pH 5.8, blue triangle) grew at a similar growth rate as UPECs grown in M9 at low pH (FIG. 8D, yellow triangle). The addition of uNGAL-Scn (5 μM) inhibited the growth of UPEC (OD₆₀₀) further particularly in the first day of culture (representative data, n=3). FIG. 8D. UPEC grown in minimal medium (M9) at different pHs showed that acidification decreasing growth rates. FIG. 8E. Real time PCR of iron acquisition genes. UPEC were grown in M9 medium and NGAL-Scn (5 μM) was added for 30 min. Note the upregulation of entA, entF, and iha genes in particular which are enterochelin synthetic and receptor proteins (n=5). FIGS. 8F,G. NGAL-Scn double fusion reporter mice were inoculated with UPEC by TU (˜1×10⁷) and imaged after 0, 0.25, 0.5 and 1 d (PhotonIMAGER optical imaging system, Biospace Labs). Kidney luciferase significantly increased compared to baseline during the course of the day (1.365±0.1420, P=0.164 at 0.25 d; 2.353±0.0003, P=0.164 at 0.5 d; 2.932±0.382 fold P<0.0001 at 1 d, n=10). FIGS. 8H-I. To determine the relative expression of uNGAL-Scn in kidney and bladder, we performed in situ hybridization of mice treated by TU with UPEC. Ngal-Scn mRNA was expressed specifically in the collecting ducts and in the thin layer of bladder uroepithelium 1 d after acute cystitis. FIG. 8J. To quantify the relative expression of Ngal-Scn, copy number was determined 1 d post-TU. There were 3 log order higher levels of Ngal-Scn message in kidney compared to bladder.

FIGS. 9A-E: NGAL (Scn) Expression in Pyelonephritis. FIG. 9A. Fluorescently labeled UPEC-GFP (green) targeted α-IC (red) of the collecting duct and bound to its apical surface (marked by v-ATPase staining) one day post-TU (n=4 mice). FIG. 9B. Paraffin in situ hybridization of C3H/HeN kidneys 1 d post TU demonstrated a pattern of Ngal-Scn positive intercalated cells (black arrows) in the distal tubules. FIG. 9C. UPEC-GFP also formed intercellular bacterial colonies in the bladder wall. FIG. 9D. Intercalated cell line Clone C²⁷ grown at high density responded to UPEC by expressing Ngal-Scn (n=4). Expression was inhibited by an NF-κB inhibitor (5 μM)¹. FIG. 9E. Primary Ngal-Luciferase2/mCherry medullary cells responded to co-culture with UPEC (3 h). Treatment with gentamicin at the time of the addition of UPEC blunted NGAL-Luc2 expression (n=3).

FIGS. 10A-D. The kidney is the dominant source of uNGAL-Scn driven by TLR activation in pyelonephritis. TLR4 dependence of kidney NGAL-Scn. FIG. 10A. Lpsⁿ control (n=9) and Lps^{d} mice (n=15) were challenged with acute pyelonephritis (20 μl of 5×10⁸ CFU ml⁻¹ CFT073). uCFU were significantly higher (P=0.0100) and FIG. 10B. kidney Ngal-Scn was significantly reduced (˜20 fold) in Lps^{d} compared with Lpsⁿ mice. FIG. 10C. Lps^{d} kidney demonstrated reduced cytokine and chemokine expression after TU. Some of the factors are known to amplify NGAL-Scn induction through NF-κB (Lpsⁿn=9 and Lps^{d} n=15). FIG. 10D. To validate the origin of uNGAL-Scn we performed reciprocal cross-transplants of Lps^{d} kidneys in Lpsⁿ hosts and vice versa. Lps^{d} kidneys expressed significantly less Ngal-Scn than Lpsⁿ mice (p<0.0005) upon challenge with LPS (1 mg kg⁻¹). Lpsⁿ kidney in the Lps^{d} host demonstrated a 15.6±2.3 fold increase in Ngal-Scn expression compared to the untreated cross transplant, while the Lps^{d} kidney in the Lpsⁿ host demonstrated only a 4.5±2.6 fold increase in Ngal-Scn RNA expression (n=3, each).

FIGS. 11A-F. uNGAL-Scn in patients with urinary infection. The cohort came from a multicenter study of kidney disease and a local clinic. FIG. 11A. uNGAL-Scn levels in patients without kidney disease were compared: Leuk Esterase Negative (LE−), Culture Negative (Cx−), n=523 vs Leuk Esterase Positive (LE+), Culture Positive (Cx+), n=43. Values were log-transformed prior to analysis. Students T test was used to compare the means of each group. FIG. 11B. In another analysis, when patients were not excluded because of other illnesses (n=1635), uNGAL-Scn expression significantly paralleled CFUs in a dose responsive fashion. Urine from patients with LE+ and 10⁴−10⁵ CFUs, and LE+ and >10⁵ CFU compared to LE+ and Cx− patient urine demonstrated significantly different levels of uNGAL-Scn (10⁴-10⁵ vs >10⁵ CFU, P=0.022; 10⁴−10⁵ vs LE+Cx−, P=0.013; >10⁵ CFU vs LE+Cx−, P<0.001). FIG. 11C. In patients presenting with urinary infections (LE+, Cx+n=141) with speciation data, those with Gram− (254.48±275.60 ng ml⁻¹, n=21) had higher levels of uNGAL-Scn than patients with Gram+ bacterial infections (50.00±84.03 ng ml⁻¹ n=5, P=0.05). FIGS. 11D-F. Patients presenting to a walk-in clinic with clinical symptoms of urinary infection had elevated uNGAL-Scn levels (611.0 ng NGAL-Scn±48.2, n=3) which were significantly depressed (P=0.008) after the application of antibiotics (77.67 ng NGAL-Scn±31.29, n=3 (FIG. 11D). Similar data were obtained after uNGAL-Scn levels were normalized by urine creatinine (FIG. 11E). Western blot detection of uNGAL-Scn demonstrated that Gram− infected patients expressed uNGAL-Scn that was sensitive to antibiotic treatment whereas Gram+ patients had substantially less uNGAL-Scn. A number of forms of NGAL-Scn are present including the monomer (25 KDa) from epithelia and complexes from neutrophils (50 KDa and 135 KDa, FIG. 11F).

FIGS. 12A-C. Generation of the Ngal-Scn^{loxP/loxP} conditional knockout murine model. FIG. 12A. A loxP site was inserted into intron 1 (yellow arrowhead), and a neomycin cassette (light blue box) was inserted into intron 5 flanked by FRT (red arrowhead) and loxP sites. The neomycin cassette was excised by crossing Ngal-Scn^{loxPneo/+} with FLP Recombinase (FLP) mice⁴⁶ generating Ngal-Scn^{loxp/+}. Ngal-Scn^{loxp/+} was bred against C57BL/6 animals to remove the FLP gene and then subsequently bred with EIIa-Cre mice for site-specific recombination⁴⁷ between the intron 1 loxP site and the intron 5 loxP site creating Ngal-Scn^{loxP/+}, Ella-Cre mice. FIG. 12B. Genotyping strategy of the Ngal-Scn loxP-cre system. Note that genotyping the targeted allele with L5F and A4R primer pairs amplified only the wild type allele (250 bp) because the PCR parameters do not amplify across the Neomycin gene. FIG. 12C. Recombination in the Cre-deleted allele was authenticated by sequencing the PCR product of Lox2F-A4R primers demonstrating the correct recombinant sequence at the loxP site.

FIGS. 13A-B. Synergy between acidification and NGAL (FIG. 13A) UPEC grown in M9 at pH 5.8 (typical urine pH) demonstrated robust inhibition with the addition of NGAL 5 μM (FIG. 13B) whereas M9 at pH 7 demonstrated reduced (and variable) growth inhibition (n=3).

FIG. 14. UPEC iron acquisition genes are modulated by DFO (5 μM in M9 media; n=5).

FIGS. 15A-D. NGAL-Luc2 signal emanates from kidneys during a UTI. Co-registration of kidney NGAL-Luc2 radiance (FIG. 15A, black circle) with NanoCT imaging in stacked (FIG. 15B) and coronal images (FIG. 15C) 1 d after a TU challenge with UPECs (1×10⁷). The Luc2 signal originated from 3-7 mm below the dorsal surface consistent with the location of the kidney. Images were collected by a three-dimensional reconstruction of NGAL-Luc2 bioluminescence (FIG. 15D).

FIG. 16. TU inoculation of L2mC double fusion reporter mice with heat-killed UPECs (˜1×10⁷) or with PBS. The heat killed bacteria induced kidney NGAL-Scn-Luc2 expression (PBS: 1.18×10⁵ vs Heat Killed Bacteria: 9.84×10⁵ and 5.71×10⁵ average radiance per kidney). Mice were imaged with the PhotonIMAGER optical imaging system (Biospace Labs) 24 h after TU (n=3).

FIGS. 17A-B. C57/BL6 mice challenged with i.p. LPS (1 mg kg⁻¹) were explanted and media collected at 12 h and 24 hr of culture. FIG. 17A. NGAL-Scn was upregulated 2 and 5 fold, 12 h and 24 h after bladder explant from LPS or from Control treated mice (n=10). FIG. 17B. NGAL-Scn was detected by Western blot. We loaded 100% of media conditioned by 6 bladders, demonstrating that the explanted bladders produced nanogram quantities of NGAL-Scn.

FIG. 18. Recovery of bacteria from the kidney and bladder 1 d post-TU inoculation of CFT073 (20 μl of 5×10⁸ CFU ml⁻¹, i.e. approximately 1×10⁷ CFU) in the C57/BL6 cystitis model (n=4), or alternatively 1 d post IP injection of CFT073 in C57/BL6 mice. Note that bacterial counts in the kidney were near or below our limit of detection in the TU inoculation (kidney LOD=10^{2,} green hashed line; bladder LOD=10³ red dotted line). IP inoculation served as a positive control.

FIGS. 19A-B. UPEC-GFP were generated by expression of pGFPuv under control of the E. coli lacZ promoter. FIG. 19A. UPEC-GFP visible on LB agar plates. FIG. 19B. Rod shaped UPEC-GFP were seen under high power (100x).

FIG. 20. Ngal-Scn message was detected in alternating cells in the kidney medulla after exposure of mice to LPS (1 mg kg⁻¹) in vivo.

### DETAILED DESCRIPTION

The present invention is based, in part, on certain discoveries which are described more fully in the Examples section of the present application. For example, the present invention is based, in part, on the discovery that, in response to infection of the urinary tract with enterochelin-dependent uropathogenic bacteria, epithelial cells of the genitourinary tract secrete NGAL protein, which has bacteriostatic activity and inhibits growth of the bacteria. The present invention is also based, in part, on the elucidation of the biochemical pathways that result in the secretion of NGAL protein by the epithelial cells of the genitourinary tract in response to a urinary tract infection.

In some embodiments, the present invention provides methods for treating or preventing infection of the urinary tract or urosepsis in a subject, the methods comprising administering to the subject a therapeutically effective amount of one or more agents selected from the group consisting of: (a) an agent that stimulates genito-urinary tract epithelial cells of the subject to secrete NGAL protein, (b) NGAL, or a functional derivative thereof, and (c) combinations of one or more thereof. In other embodiments, the present invention provides methods for treating or preventing infection of the urinary tract or urosepsis in a subject, the methods comprising stimulating genito-urinary tract epithelial cells of the subject to secrete NGAL protein. In further embodiments, the present invention provides pharmaceutical compositions for use in treating a urinary tract infections and/or urosepsis. These and other aspects of the present invention are described in more detail in this “Detailed Description” section of the application, and also in the Summary of the Invention, Examples, Drawings, and Claims sections of the application.

### Abbreviations and Definitions

The abbreviation “NGAL” refers to Neutrophil Gelatinase Associated Lipocalin. NGAL is also referred to in the art as human neutrophil lipocalin, siderocalin, α-micropglobulin related protein, Scn-NGAL, lipocalin 2, 24p3, superinducible protein 24 (SIP24), uterocalin, and neu-related lipocalin. These alternative names for NGAL may be used interchangeably herein. Unless stated otherwise, the terms “NGAL” and “Ngal” as used herein, includes any NGAL protein, or functional derivative thereof. The terms “functional derivative” or “functional derivatives thereof,” as they are used herein in relation to NGAL, refer to any fragments, variants, mutants or analogs of NGAL that retain the ability to bind to iron, including, but not limited to iron bound to siderophores, and/or retain bacteriostatic activity. Functional derivative of NGAL include, but are not limited to, mutated versions of the NGAL protein, and chemically modified versions of the NGAL protein. Such functional derivatives may have one or more amino acids or other chemical moieties added, removed, or substituted. The term “analog” includes structural equivalentd or mimetics, as understood by those of skill in the art. In some embodiments the NGAL protein is wild-type NGAL, such as wild-type human NGAL. In some contexts, the term NGAL may also be used herein to refer to a nucleotide that encodes an NGAL protein, or a functional derivative thereof, such as a DNA or RNA molecule that encodes an NGAL protein.

The abbreviation “uNGAL” is an abbreviation for urinary NGAL and refers to NGAL that is found in the urine or elsewhere in the genito-urinary tract, or that is expressed by cells of the genito-urinary tract.

The abbreviation “UTI” refers to a urinary tract infection.

The abbreviation “UPEC” refers to a uropathogenic Eschericia coli—a type of bacterium.

The abbreviation “E. coli” refers to a the bacterium Eschericia coli.

The abbreviation “CFU” refers to colony-forming units, for example of a bacterium. The abbreviation “uCFU” refers to urinary colony-forming units, for example of a urinary or urinary tract bacterium.

The abbreviation “TU” refers to trans-urethral.

The abbreviations “IP” and “i.p.” refer to intraperitoneal.

The abbreviation “KO” refers to knock-out or knock-out organism (e.g. mouse).

The abbreviation “CKO” refers to conditional knock-out or conditional knock-out organism (e.g. mouse).

The abbreviation “WT” refers to wild-type.

The abbreviation “GU” refers to genitor-urinary.

The abbreviation “CD” refers to the collecting duct of the kidney.

The abbreviation “TAL” refers to the tall ascending limb of Henlé in the kidney.

The abbreviation “GFR” refers to the glomerular filtration rate of the kidney.

The abbreviation “PCR” refers to polymerase chain reaction.

The abbreviation “QPCR” refers to a quantitative polymerase chain reaction.

The term “urosepsis” is used herein in accordance with its normal meaning in clinical medicine, and refers to bacteremia that is secondary to a UTI.

The abbreviation “AKI” refers to acute kidney injury.

The abbreviation “NRF2” refers to nuclear factor (erythroid-derived 2)-like 2, which is also known as NFE2L2 and Nrf2.

The abbreviation “HIF” refers to hypoxia inducible factor.

The abbreviation “NF-κB” refers to nuclear factor kappa-light-chain-enhancer of activated B cells.

The phrases “pharmaceutically acceptable,” “pharmacologically acceptable,” and “physiologically acceptable” refer to molecular entities and compositions that do not produce an adverse, allergic or other untoward reaction when administered to an animal, such as, for example, a human.

The phrase “pharmaceutically acceptable carrier” as used herein means a material, composition or vehicle, such as a liquid or solid filler, diluent, excipient, solvent or encapsulating material, that can be used in a composition of the invention without adversely affecting the biological activity of the active ingredient(s) of the compostions, such as NGAL. Each carrier should be “acceptable” in the sense of being compatible with other ingredients of the composition, including the active ingredients, such as NGAL, and not injurious to the subject. Some examples of materials which can serve as pharmaceutically-acceptable carriers include, but are not limited to: any and all solvents, dispersion media, coatings, surfactants, antioxidants, preservatives, isotonic agents, absorption delaying agents, salts, preservatives, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredients of the compositions described herein, such as NGAL, its use in the therapeutic or pharmaceutical compositions is contemplated.

The phrase “therapeutically effective amount” refers to an amount of the active ingredient of a compositions described herein, such as NGAL, that is effective to produce beneficial results, particularly with respect to the treatment, prevention or amelioration of UTI or urosepsis, as described herein, in the recipient, such as an animal or a human patient. Such amounts can readily be determined by those of ordinary skill in the art, for example on the basis of published literature, in vitro testing, or by conducting studies in animals or in human subjects.

A “patient”, “recipient”, or “subject” means an animal or organism, such as a warm-blooded animal or organism. Illustrative animals include, without limitation, mammals, for example, humans, non-human primates, pigs, cats, dogs, rodents, horses, cattle, sheep, goats and cows. The invention is particularly suitable for human patients and subjects.

The words “a” and “an” as used herein refers to “one or more”. More specifically, the use of “comprising,” “having,” or other open language in claims that claim a combination or method employing an object, denotes that “one or more of the object” can be employed in the claimed method or combination.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

NGAL

NGAL protein, or functional derivatives thereof, can be manufactured by any suitable method known in the art for manufacture of protein drugs. For example NGAL protein can be made using standard techniques known for the production of recombinant proteins, for example by delivering to a cell, such as a bacterial cell or a mammalian cell, an expression vector containing a nucleotide sequence that encodes an NGAL protein under the control of a suitable promoter, and culturing the cell under conditions in which the protein will be expressed. Nucleotide sequences that encode NGAL proteins are well known in the art. Methods for the large scale culture, isolation, and purification of recombinant proteins are well known in the art and can be used in the manufacture of the NGAL proteins of the present invention. Similarly, methods of producing peptides and proteins synthetically are known in the art and can be used in the manufacture of the NGAL proteins of the present invention.

In certain embodiments, the NGAL proteins, or functional derivatives thereof, may be used as fusion proteins comprising the NGAL protein and one or more additional “tags.” Such additional tags can be fused to the N- or C-terminus of the NGAL proteins, or can in some instances be added at an internal location to the extent that the inclusion of the tag does not adversely affect the function of the NGAL protein. Suitable tags include, but are not limited to glutathione-S-transferase (GST), poly-histidine (H is), alkaline phosphatase (AP), horseradish peroxidase (HRP), and green fluorescent protein (GFP). Other suitable tags will also be apparent to those skilled in the art. The tags may be useful for several applications, including to assist in the isolation and/or purification of the NGAL proteins and/or to facilitate their detection.

Many chemical modifications of proteins are known in the art to be useful for improving the properties of protein-based drugs and such modifications can be used in accordance with the present invention to improve the stability and reduce the immunogenicity of the NGAL proteins of the invention for therapeutic applications. For example, it is well known in the art that the process of covalent attachment of polyethylene glycol polymer chains to another molecule (i.e. PEGylation) can “mask” a proteinaceous agent from the host's immune system, and also increase the hydrodynamic size (size in solution), prolong the circulatory half-life, and improve water solubility of protein-based drugs. Various other chemical modifications are also known and used in the art and can be used in conjunction with the NGAL proteins of the invention.

In some embodiments, it may also be desirable to use a complex containing an NGAL protein and a siderophore, such as enterochelin, or a derivative or variant thereof. Such complexes can readily be prepared using standard methodologies known in the art. For example, an NGAL-siderophore complex can be prepared by mixing NGAL and a siderophore together in a molar ratio of 1:1 (e.g. enterochelin) or 1:3 (e.g. catechol). The mixture can be incubated at room temperature for a suitable time, e.g. 30 minutes, to allow for complex formation. Unbound siderophore can then be removed/separated from the bound siderophore-NGAL complexes using standard separation techniques, such as centrifugation based techniques, filter-based techniques, or other size-based separation techniques. Siderophores that are known in the art include, but are not limited to enterochelin, TRENCAM, MECAM, TRENCAM-3,2-HOPO, parabactin, carboxymycobactin, fusigen, triacetylfusarinine, feriichrome, coprogen, rhodotorulic acid, ornibactin, exochelin, ferrioxamine, desferrioxamine B, aerobactin, ferrichrome, rhizoferrin, pyochelin, pyoverdin. The structures of these compounds are disclosed in Holmes et al., Structure, 2005, 13:29-41 and Flo et al., Nature, 2004, 432: 917-921, the contents of which are hereby incorporated by reference. Several of the above siderophores are known to bind to lipocalins, including NGAL, and complexes of these siderophores and lipocalins are known to be able to sequester iron (see for example, Holmes et al., Structure, 2005, 13:29-41 and Flo et al., Nature, 2004, 432: 917-921; Goetz et al, Molecular Cell, 2002, 10: 1033-1043 and Mori, et al., “Endocytic delivery of lipocalin-siderophore-iron complex rescues the kidney from ischemia-reperfusion injury.” J. Clin Invest., 2005, 115, 610-621). Thus, in some aspects the present invention contemplates the use and/or administration of an NGAL protein together with a siderophore, including, but not limited to, the siderophores listed herein. In preferred aspects the siderophore is selected from the group consisting of enterochelin, pyrogallol, carboxymycobactin, catechol, and variants or derivatives thereof. Any variant or derivative of such siderophores that retains the ability to bind to iron (ideally in a pH insensitive manner) and that retains the ability to bind to NGAL may be used in accordance with the present invention.

Agents that Stimulate Production of NGAL by GU Tract Epithelial Cells

In some embodiments, the present invention provides methods for treating or preventing UTI or urosepsis in a subject comprising administering to the subject an agent that stimulates production of NGAL by epithelial cells of the urinary tract. In other embodiment, the present invention provides compositions that comprise an agent that stimulates production of NGAL by epithelial cells of the urinary tract. Any suitable agent that stimulates the production of NGAL by epithelial cells of the urinary tract may be used in the methods of compositions of the invention.

In some embodiments, the present invention provides methods for treatment or prevention of UTI or urosepsis that comprise administration of an NFκB activator. In other embodiments, the present invention provides compositions that comprise an NFκB activator. Any NFκB modulator that stimulates the expression and/or secretion of NGAL by epithelial cells of the urinary tract can be used in accordance with the present invention, including, but not limited to compounds SRI#22771, SRI#22772, SRI#22773, SRI#22774, SRI#22775, SRI#22776, SRI#22777, SRI#22778, SRI#22779, SRI#22780, SRI#22781, SRI#22782, SRI#22816, SRI#22817, SRI#22818, SRI#22819, SRI#22820, and SRI#22864, as described in Manuvakhova et al., Identification of Novel Small Molecule Activators of Nuclear Factor-κB With Neuroprotective Action Via High-Throughput Screening, Journal of Neuroscience Research, 89:58-72 (2011), the contents of which are hereby incorporated by reference.

In some embodiments, the present invention provides methods for treatment or prevention of UTI or urosepsis that comprise administration of an activator of a TLR-NFκB pathway, such as the TLR4-NFκB pathway or the TLR11-NFκB pathway. In other embodiments, the present invention provides compositions that comprise an an activator of a TLR-NFκB pathway, such as the TLR4-NFκB pathway or the TLR11-NFκB pathway. Any activator of a TLR-NFκB pathway modulator that stimulates the expression and/or secretion of NGAL by epithelial cells of the urinary tract can be used in accordance with the present invention, including, but not limited to NFκB, and the TLR4 activators described in Huang et al., “Synthesis of serine-based glycolipids as potential TLR4 activators,” Org. Biomol. Chem., 2011, 9, 2492-2504, the contents of which are hereby incorporated by reference.

In some embodiments, the present invention provides methods for treatment or prevention of UTI or urosepsis that comprise administration of a lipid A derivative, an endotoxin derivative, or a lipopolysaccharide derivative. In other embodiments, the present invention provides compositions that comprise a lipid A derivative, an endotoxin derivative or a lipopolysaccharide derivative. Any lipid A derivative, endotoxin derivative, or lipopolysaccharide derivative that stimulates the expression and/or secretion of NGAL by epithelial cells of the urinary tract, and is suitable for clinical use (for example, it is not toxic) can be used in accordance with the present invention, including, but not limited to, monophosphoryl Lipid A (as described in Johnson et al., “Characterization of a nontoxic monophosphoryl lipid A,” Rev. Infect. Dis. (1987), 9 Suppl 5:S512-6), E5531 (as described in Kawata et al., “A synthetic non-toxic lipid A derivative blocks the immunobiological activities of lipopolysaccharide.” Br J. Pharmacol. 1999 June; 127(4):853-62), the lipid A derivative described in Santhanam et al., (“Preparation of a Lipid A Derivative That Contains a 27-Hydroxyoctacosanoic Acid Moiety,” Org. Lett., 2004, 6 (19), pp 3333-3336), and Lipid X, Lipid Y, “incomplete lipid A,” or monophosphoryl lipid A (TLC-3) (as described in Takayama et al.,” Separation and characterization of toxic and nontoxic forms of lipid A,” Reviews of Infectious Diseases (1984), 6(4): 439-43), the contents of each of which are hereby incorporated by reference in their entireties.

In some embodiments, the present invention provides methods for treatment or prevention of UTI or urosepsis that comprise administration of a HIF modulator. In other embodiments, the present invention provides compositions that comprise a HIF modulator. Any HIF modulator that stimulates the expression and/or secretion of NGAL by epithelial cells of the urinary tract can be used in accordance with the present invention, including, but not limited to, HIF, HIF prolyl-hydroxylase inhibitors, such as FG-2216 and FG-4592, (See, Bruegge K, Jelkmann W, Metzen E (2007). “Hydroxylation of hypoxia-inducible transcription factors and chemical compounds targeting the HIF-alpha hydroxylases”. Curr. Med. Chem. 14 (17), the contents of which are hereby incorporated by reference in its entirety), deferoxamine, desferoxamine mesylate, Desferal Mesylate®, desferri-exochelin, ciclopirox olamine [3, Loprox®, 6-cyclohexyl-1-hydroxy-4-methyl-2(1H)-pyridone 2-aminoethanol], 8-methyl-pyridoxatin, N-oxaloylglycine (NOG), DMOG (6, dimethyl-oxalylglycine), 3,4-dihydroxybenzoate, or pyridine-2,5-dicarboxylate. Other HIF modulators are described in Nagle et al., Curr. Pharm. Des. 2006; 12(21): 2673-2688, and Semenzathe et al., Drug Discovery Today, 2007, 12(19-20): 853-859, the contents of which are hereby incorporated by reference.

In some embodiments, the present invention provides methods for treatment or prevention of UTI or urosepsis that comprise administration of an NRF2 modulator. In other embodiments, the present invention provides compositions that comprise an NRF2 modulator. Any NRF2 modulator that stimulates the expression and/or secretion of NGAL by epithelial cells of the urinary tract can be used in accordance with the present invention, including, but not limited to, dithiolethione NRF2 modulators, oltipraz, oleane triterpenoid compounds, bardoxolone methyl, and reservatrol.

Urinary Tract Infections & Urosepsis

In some embodiments, the present invention provides compositions and methods for the treatment or prevention of UTI or urosepsis. In some embodiments, the UTI or urosepsis is caused by, or associated with, one or more bacterial species. In some embodiments, the UTI or urosepsis is caused by, or associated with, one or more siderophore-dependent uropathogenic bacteria, such as catecholate-dependent uropathogenic bacteria. In some embodiments, the UTI or urosepsis is caused by, or associated with, one or more enterochelin-dependent uropathogenic bacteria. In some embodiments, the UTI or urosepsis is caused by, or associated with, an E. coli infection.

Pharmaceutical Compositions & Administration

In some embodiments, the present invention provides pharmaceuctical compositions for use in treating or preventing a urinary tract infection or urosepsis. Such compositions comprising a therapeutically effective amount of an agent that stimulates genito-urinary tract epithelial cells to secrete NGAL protein, and/or a therapeutically effective amount of NGAL, or a functional derivative thereof. Examples of agents that stimulate genito-urinary tract epithelial cells to secrete NGAL protein include, but are not limited to derivatives of lipid A, derivatives of lipopolysaccharide, derivatives of endotoxin, activators of the TLR4-NFkB pathway, activators of the TLR11-NFkB pathway, activators of NFκB, NRF2 modulators, and HIF modulators. Each of these agents (including NGAL, or derivatives thereof), may be formulated into a pharmaceutical composition.

The pharmaceutical compositions of the invention include those suitable for oral or parenteral (including intramuscular, subcutaneous and intravenous) administration. Administration of a therapeutically effective amount of any of the agents described herein can be accomplished via any mode of administration suitable for therapeutic agents. One of skill in the art can readily select a suitable mode of administration without undue experimentation. Suitable modes may include systemic or local administration such as oral, nasal, parenteral, transdermal, subcutaneous, topical, intravenous (both bolus and infusion), intraperitoneal, or intramuscular administration modes. In some embodiments, oral or intravenous administration is used. In other embodiments, the compositions of the invention are administered directly to the desired site of action, such as for example, the urinary tract, for example by local injection or local infusion or by use of (e.g. conjugation to) agents useful for targeting proteins or pharmaceuticals to specific tissues, such as antibodies etc. In some embodiments, the compositions of the invention are administered directly to the kidney or elsewhere in the genitourinary tract, for example by transurethral (TU) delivery. In some embodiments the compositions of the invention are administered directly to the genitourinary tract using a catheter or similar medical device. For example, in cases where a catheter is to be inserted into a subject transurethrally, for example in the course of a medical procedure, it may be desirable to deliver the compositions of the invention transurethrally prophylactically to the subject, to prevent or mitigate the effects of any UTI that could otherwise be caused as a result of the medical procedure.

Depending on the intended mode of administration, the agents of the invention may be in solid, semi-solid or liquid dosage form, such as, for example, injectables, tablets, suppositories, pills, time-release capsules, elixirs, tinctures, emulsions, syrups, powders, liquids, gels, creams, suspensions, or the like. In one embodiment the agents of the invention may be formulated in unit dosage forms, consistent with conventional pharmaceutical practices. Liquid, particularly injectable, compositions can, for example, be prepared by dissolution or dispersion. For example, agents of the invention can be admixed with a pharmaceutically acceptable solvent such as, for example, water, saline, aqueous dextrose, glycerol, ethanol, and the like, to thereby form an injectable isotonic solution or suspension.

Parental injectable administration can be used for subcutaneous, intramuscular or intravenous injections and infusions. Injectables can be prepared in conventional forms, either as liquid solutions or suspensions or solid forms suitable for dissolving in liquid prior to injection. One embodiment, for parenteral administration, employs the implantation of a slow-release or sustained-released system, according to U.S. Pat. No. 3,710,795, incorporated herein by reference.

Compositions comprising the agents of the invention can be sterilized and may contain any suitable adjuvants, preservatives, stabilizers, wetting agents, emulsifying agents, solution promoters, salts (e.g. for regulating the osmotic pressure), pH buffering agents, and/or other pharmaceutically acceptable substances, including, but not limited to, sodium acetate or triethanolamine oleate. In addition, the compositions of the invention may also contain other therapeutically useful substances, such as, for example, other iron chelators or other bacteriostatic or antibacterial agents. In some embodiment, the compositions of the invention may comprise on or more additional agents that are useful for the treatment or prevention of UTI or urosepsis, such as bacteriostatic agents and antibiotics that are useful in the treatment of UTI or urosepsis. For example, such an addition agents may be a bacteriostatic agent or antibiotics that is effective to inhibit the growth of uropathogenic E. coli (UPEC) strains, including enterochelin-dependent UPECs.

The methods of treatment provided herein may also comprise treatment with a bacteriostatic agent and/or antibiotic that is useful in the treatment of UTI or urosepsis—in addition to: (a) NGAL, or a functional derivative thereof, or (b) an agent that stimulates the production of NGAL by urinary tract epithelial cells, or (c) any combination thereof. For example, such an additional agent may be a bacteriostatic agent or antibiotic that is effective to inhibit the growth of uropathogenic E. coli (UPEC) strains, including enterochelin-dependent UPECs.

The compositions of the invention can be prepared according to conventional mixing, granulating or coating methods, respectively, and the present pharmaceutical compositions can contain from about 0.1% to about 99%, preferably from about 1% to about 70% of the composition of the invention by weight or volume.

The dose and dosage regimen to be used in accordance with the methods of treatment of the invention can be determined in accordance with a variety of factors including the species, age, weight, sex and medical condition of the subject; the severity of the condition; the route of administration; and the renal or hepatic function of the subject. A person skilled in the art can readily determine and/or prescribe an effective amount of an agent of the invention useful for treating or preventing UTI or urosepsis, for example, taking into account the factors described above. Dosage strategies are also provided in L. S. Goodman, et al., The Pharmacological Basis of Therapeutics, 201-26 (5th ed.1975), which is herein incorporated by reference in its entirety. In one embodiment, compositions of the invention are administered such that the active agent(s) is administered at a dose range of about 1 to about 100 mg/kg body weight, and typically at a dosage of about 1 to about 10 mg/kg body weight, or is administered at a dose that results in a concentration in the range of about 0.1 ng/ml to about 100 ng/ml, e.g., in the range of about 1.0 ng/ml to about 20 ng/ml, in the blood or locally at the site of action, such as in the urinary tract.

Screening Methods

The present invention provides methods of screening for agents that stimulate epithelial cells of the urinary tract, such as kidney epithelial cells (including epithelial cells of the collecting ducts or of the thick ascending limb of Henlé), bladder epithelial cells, and urethral epithelial cells, to produce NGAL mRNA or protein. In some embodiments, such screening methods comprise providing a population of urinary tract epithelial cells, contacting the population of urinary tract epithelial cells with one or more test agents, and testing for production of NGAL mRNA or protein by the urinary tract epithelial cells, thereby identifying agents that stimulate production of NGAL mRNA or protein by the urinary tract epithelial cells. In one embodiment, the present invention provides a method of identifying an agent that stimulates epithelial cells of the urinary tract to produce NGAL mRNA or NGAL protein, the method comprising: (a) providing a test population of urinary tract epithelial cells and a control population of urinary tract epithelial cells, (b) contacting the test population of urinary tract epithelial cells with one or more test agents, (c) contacting the control population of urinary tract epithelial cells with no agent or with one or more negative control agents, and (d) determining the level of NGAL mRNA or NGAL protein in the test population and the control population, or in a culture supernatant thereof, wherein a level of NGAL mRNA or NGAL protein in the test population, or a culture supernatant thereof, that is higher than the level of NGAL mRNA or NGAL protein in the control population, or a culture supernatant thereof, indicates that the test agent is an agent that stimulates production of NGAL mRNA or NGAL protein by the urinary tract epithelial cells. In another embodiment, the present invention provides a method of identifying an agent that stimulates epithelial cells of the urinary tract to produce NGAL mRNA or NGAL protein, the method comprising: (a) providing a population of urinary tract epithelial cells, (b) determining the control level of NGAL mRNA or NGAL protein in the population of urinary tract epithelial cells, or in a culture supernatant thereof, wherein the control level is the level of NGAL mRNA or NGAL protein present prior to contacting the urinary tract epithelial cells with one or more test agents, (c) contacting the urinary tract epithelial cells with one or more test agents, (d) determining the test level of NGAL mRNA or NGAL protein in the population of urinary tract epithelial cells, or in a culture supernatant thereof, wherein the test level is the level of NGAL mRNA or NGAL protein present subsequent to contacting the urinary tract epithelial cells with the one or more test agents, wherein if the test level of NGAL mRNA or NGAL protein exceeds the control level of NGAL mRNA or NGAL protein, the test agent is an agent that stimulates production of NGAL mRNA or NGAL protein by the urinary tract epithelial cells.

In some such embodiments, the urinary tract epithelial cells may be in vivo, for example in a mouse model. In other embodiments, the urinary tract epithelial cells may be cultured in vitro. Urinary tract epithelial cells that are cultured in vitro may be primary cultures, or may be derived from primary cultures, or may be cell lines, such as established urinary tract epithelial cell lines, including kidney epithelial cell lines, bladder epithelial cells lines, urethral epithelial cell lines, and the like. The test agents may be any suitable test agents, including, but not limited to, libraries of small molecule drugs, libraries of proteinaceous or peptide drugs (including peptidomimetic drugs), libraries of antibodies, libraries of RNA molecules (including, but not limited to, antisense RNAs, siRNAs, shRNAs, and microRNAs, ribozymes), and the like. In addition to libraries of test agents, individual test agents, or smaller populations of test agents, may also be used. Any suitable negative controls can be used. For example, the epithelial cells may be contacted with no test agent, or with an inactive agent, such as an agent that is known not to stimulate NGAL production. Any suitable positive control may be used. For example, an agent that is known to stimulate production of NGAL by urinary tract epithelial cells, such as, for example, Lipid A. Any suitable means may be used to detect NGAL production by the urinary tract epithelial cells. In one embodiment, secreted NGAL protein is detected in cell supernatants. In another embodiment, NGAL protein within the epithelial cells is detected. NGAL protein may be detected using any suitable means. In one embedment, NGAL protein is detected using an antibody to NGAL. The NGAL antibody may be labeled with a detectable moiety, or a secondary antibody that is labeled with a detectable moiety may be used. Suitable detectable moieties may include enzyme subtstrates (such as horseradish peroxidase, alkaline phosphatase, and the like), and fluorescent labels (such as green fluorescent protein, and the like). In one embodiment NGAL protein may be detected in an ELISA assay using an anti-NGAL antibody. In another embodiment NGAL mRNA is detected. NGAL mRNA may be detected using any suitable means, including, but not limited to, in situ hybridization, Northern blotting, PCR, QPCR, and the like. Any suitable probes or primers for detection of NGAL mRNA may be used.

Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXAMPLES

### Example 1

The numbers in superscript below refer to the corresponding numbered reference(s) at the end of this Example.

UTIs are one of the most prevalent and resource taxing diseases with 13.3% (12.8 million) of all women and 2.3% (2 million) of men in the USA are infected annually producing an annual cost of $3.5 billion for evaluation and treatment'. In 2000 there were an estimated 11.02 million visits (2.05 million men; 8.97 million women) to a physician's office or hospital with UTI listed as any diagnosis.¹ Uropathogenic E. coli (UPEC) represent 70-95% of all cases of UTI and many of these bacteria rely on catecholate-siderophores as their primary iron uptake mechanism.¹ Urine dipsticks are used to read the biochemical signature of a UTI. Dipsticks recognize the presence of leukocyte esterase and nitrite in the urine. Leukocyte esterase corresponds to pyuria and nitrite reflects the presence of Enterobacteriaceae, which convert urinary nitrate to nitrite.^{3,4} In a review of six studies including women aged 17 to 70 with suspected UTI in primary care settings, positive dipstick findings (nitrite or leukocyte esterase and blood) had sensitivity and specificity of 75 and 66 percent, respectively⁵ and in children they are at best 88 percent sensitive.⁶

Neutrophil Gelantinase Associated Lipocalin (NGAL) is a secreted lipocalin which is markedly upregulated and expressed by the kidney human adults⁷ and children⁸, as well as in mice⁹⁻¹¹, rats, and pigs in proportion to the dose of stimuli such as ischemia-reperfusion (I/R)⁹, hypoxia, drug toxicity, and bacterial infection¹⁰⁻¹³ which typically generate kidney damage. However it has not been clear why this protein is expressed in the urinary system after kidney damage of different types. NGAL is a bacteriostatic protein₁₄ by binding catecholate-siderophore¹⁵ which sequesters iron from bacteria. The studies described in this Example relate to the relationship between NGAL expression by the kidney and lower urinary tract infection.

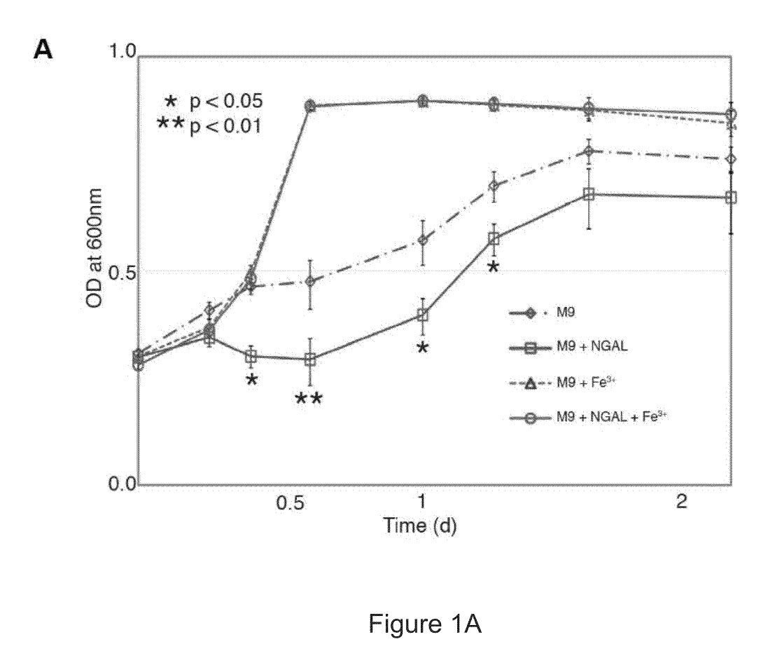

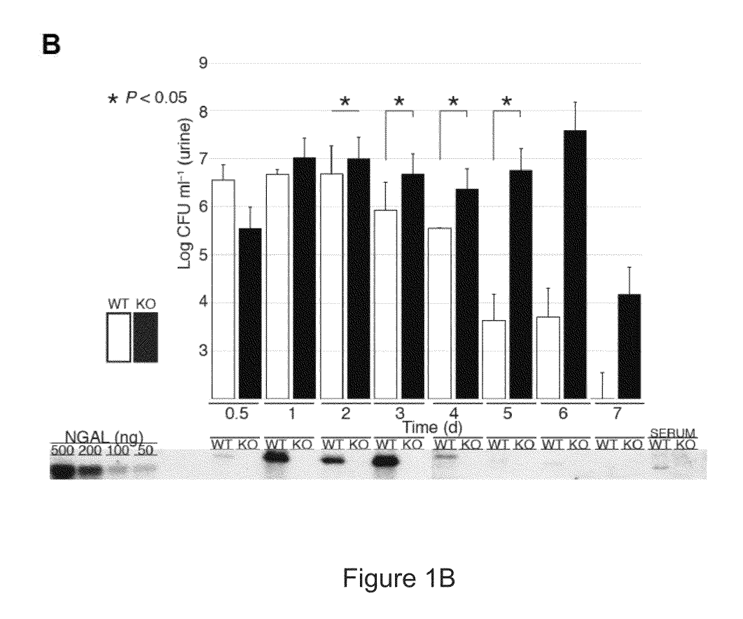

To investigate the relationship between NGAL and UPEC a pyelonephritogenic clinical isolate of uropathogenic Escherichia coli, CFT073, was aliquoted in 96 well plates containing M9 minimal medium supplemented with MgCl₂ and glucose (see green hashed line: FIG. 1a), and bacterial growth was monitored by spectrophotometry. Bacterial growth was significantly inhibited (FIG. 1a) when the UPEC was grown with the addition of mouse (m) NGAL (red line and open red squares: 5 μM), but it could be rescued by the addition of FeCl₃ (blue line with open blue circles: 1 mM). This data is consistent with NGAL's activity in iron scavenging.¹⁵

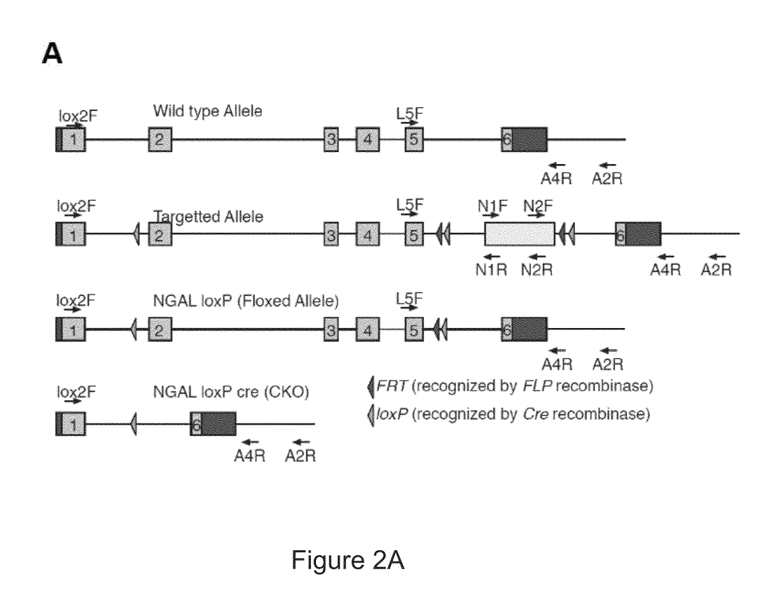

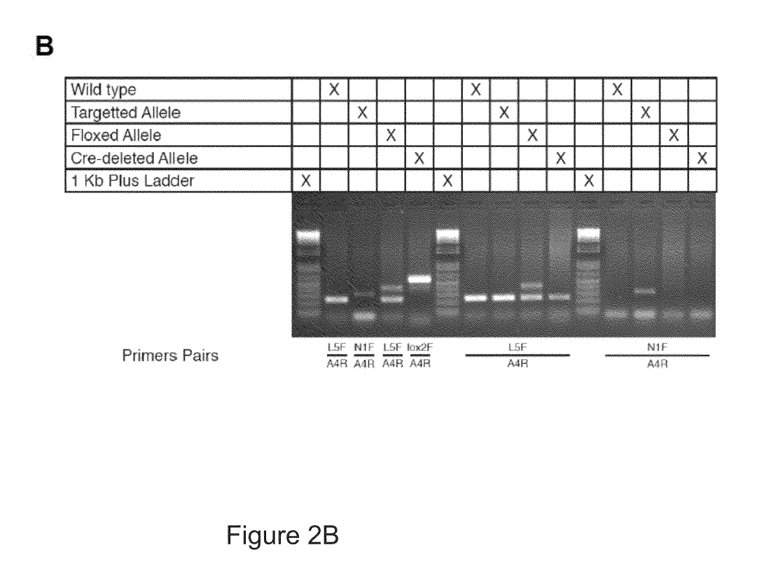

To determine the role NGAL has in an acute UTI in vivo a conditional NGALloxP/loxP animal and tissue specific knockouts were generated (Methods and FIG. 2). The NGALloxP/loxP mouse was bred with EIIa-Cre16 mouse (NGALloxP/loxP, EIIa-Cre) which generated a knock-out of NGAL in all cells. NGAL wild-type (Ngal+/+) mice (white bar) and NgalloxP/loxP, EIIa-Cre (black bars) mice were challenged with a transurethral (TU) infection of the UPEC (10-20 μl of 5×10⁹ CFU/ml CFT073) and urinary (u) NGAL and urinary colony forming units (uCFU) were monitored for one week (FIG. 1b). In Ngal+/+ mice we noted that the secretion of uNGAL mirrored both the onset of the urinary tract infection (UTI), indicated by log order increases in uCFU, and the resolution of the acute phase of the infection (˜day 3-4, FIG. 1b). NGAL+/+ animals with the acute UTI cleared the CFT073 bacteria within 3-5 days after the initial challenge with the UPEC, but animals without a functioning NGAL allele took significantly more time (6 days) than the wild type mice to clear the UPEC (NgalloxP/loxP, EIIa-Cre mice had a log order more uCFU than Ngal+/+ at 2, 3, 4 and 5 days post-TU, p<0.05, UTest: FIG. 1b). Hence, uNGAL is necessary to rapidly clear an acute UTI by an enterochelin dependent UPEC and the NGAL protein itself is sufficient for bacteriostasis.

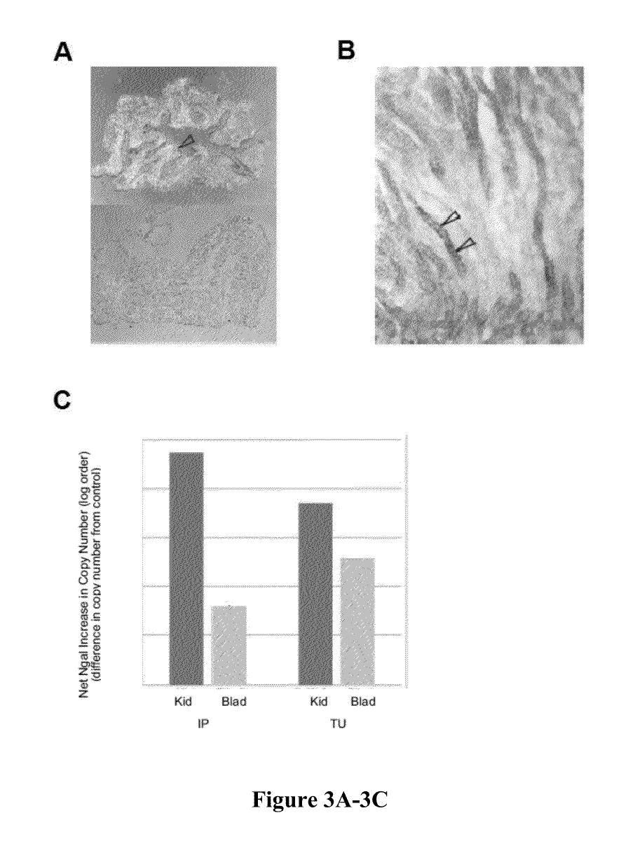

To determine the normal cellular source of uNGAL in response an acute UPEC infection, in situ hybridization was performed in NGAL+/+ kidneys 1 day post-TU injection of the UPEC (5×10⁹ CFU/ml). High levels of NGAL RNA were expressed by bladder epithelium (FIG. 3a) and through microscopic examination of the kidney, NGAL mRNA was identified in the epithelia of the Thick Ascending Limb of Henle (TAL) and the Collecting Ducts (CD)(FIG. 3b). To determine the relative contribution of these organs to uNGAL production, QPCR was performed and the copy number of NGAL per organ 1 day post-TU challenge with the UPEC was determined. IP injection was used as a control to stimulate NGAL expression. With either systemic or transurethral application of bacteria, the kidney was the major contributor to uNGAL as measured by the number of copies of NGAL respective of total RNA (FIG. 3c).

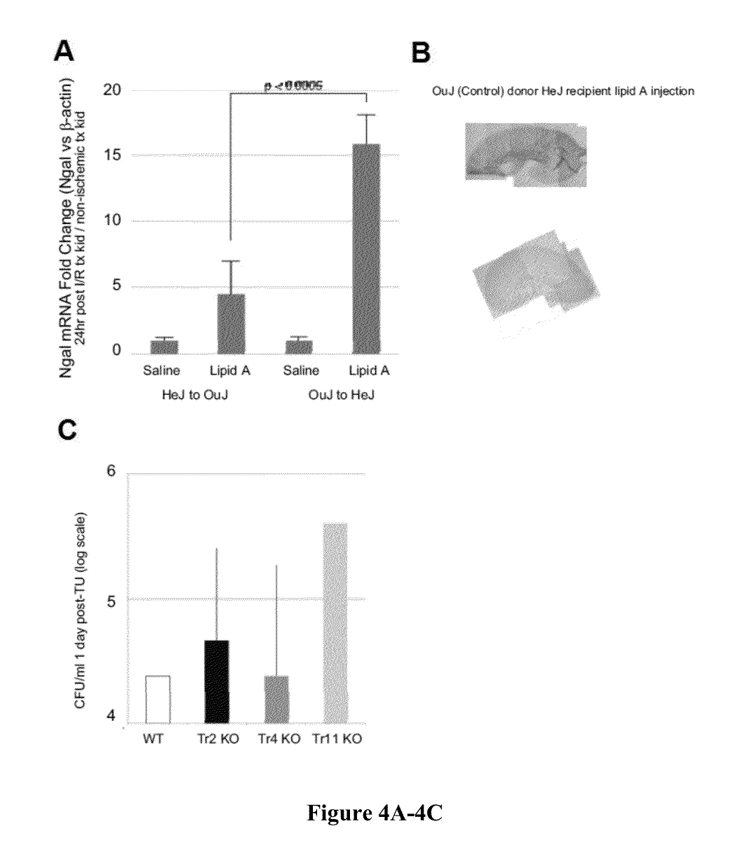

The data showed that both the kidney and the bladder might contribute to the uNGAL pool. NGAL expression was further dissected by selectively knocking out NGAL in different segments of the GU tract. NGAL was first knocked out in the collecting duct (CD) by generating a NGALloxP/loxP, HoxB7/cre19 (NgalloxP/loxP, HoxB7/cre) CKO mouse. HoxB7/cre has been noted to be expressed in the ureteric epithelium of the distal, non-branching medullary collecting ducts and the epithelium of the ureter¹⁹. It was found that the HoxB7 compartment was a major contributor to uNGAL because there was greater than a log order increase in median uCFU after a TU challenge with the UPEC compared to the wild type mice (FIG. 4). These data support findings that NGAL expression was localized after urinary infection and was localized to the collecting duct⁹. Therefore many types of GU epithelia generate uNGAL protein. The combination of in situ hybridization, organ copy number analysis, and segment specific knockouts indicated in vivo that the major source of NGAL RNA is the kidney and the major source of the NGAL protein is also the kidney.

Previous findings⁹ from isolated primary cells revealed that bacterial gram-negative components, such as lipid A, bind to Toll-like receptors (TLRs), such as Toll-like receptor 4 (TLR4), and activate NF-κB⁸ which induces NGAL in vitro. To determine whether uNGAL originated from the kidney in a TLR-dependent fashion, kidney transplantation between Tlr-4-mutant CH3/HeJ mice and wild type CH3/HeOuJ mice was performed. To evaluate the contribution of TLR signaling to NGAL expression a cross-transplant model using TLR mutants and systemic administration of LPS as a positive control was used. The CH3/HeJ kidneys from the LPS-insensitive mice were transplanted into CH3/HeOuJ LPS-sensitive (control) mouse bodies, and vice versa. Two weeks after graft maturation, when uNGAL and sCr had stabilized to normal values, a low dose of lipid A (1 mg/kg of body weight) was administered to induce Ngal expression in the kidney (FIG. 4a). QPCR revealed that the wild type kidney in the Tlr-4-mutant body had a 15.6±2.3 fold increase in Ngal expression while the Tlr-4-mutant kidney in the wild type body had only a 4.5±2.6 fold increase in Ngal expression. It is not surprising that there was some Ngal induction in the knockout (KO) kidney because CH3/HeJ are partial KOs and it has also been previously shown that lipid A can signal through the MyD88-dependent pathway without a functioning Tlr4. These results suggests that TLR4 is the receptor to lipid A in the kidney, and indicate that NGAL is induced by LPS activation of the TLR4::NfKB pathway.

The kidney can also sense a bacterial infection by the presence of necrotic cell debris from endogenous and endogenous origin. It has been shown that TLR4 is activated by heat-shock proteins, fibronectin, hyaluronic acid, heparin sulfate and fibrinogen,²⁰⁻²⁶ suggesting that the kidney can gauge the early onset of a bacterial infection in the blood and the bladder. TLR4 can bind to a myriad of factors, and this single-pass transmembrane receptor can elicit a tightly regulated signal transduction pathway from various molecules.

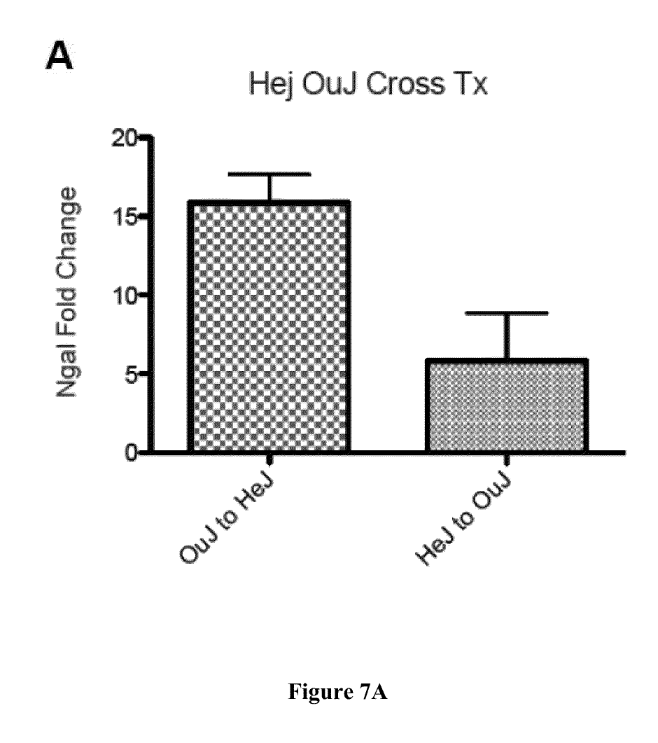

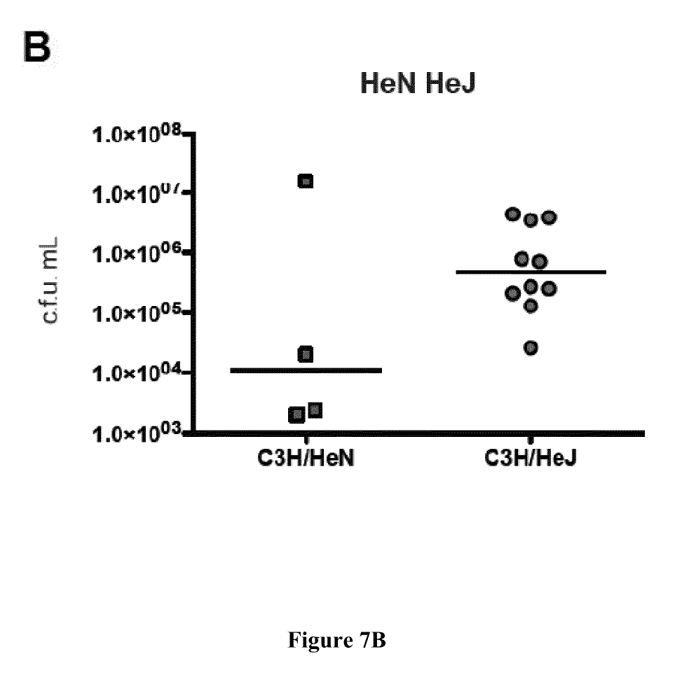

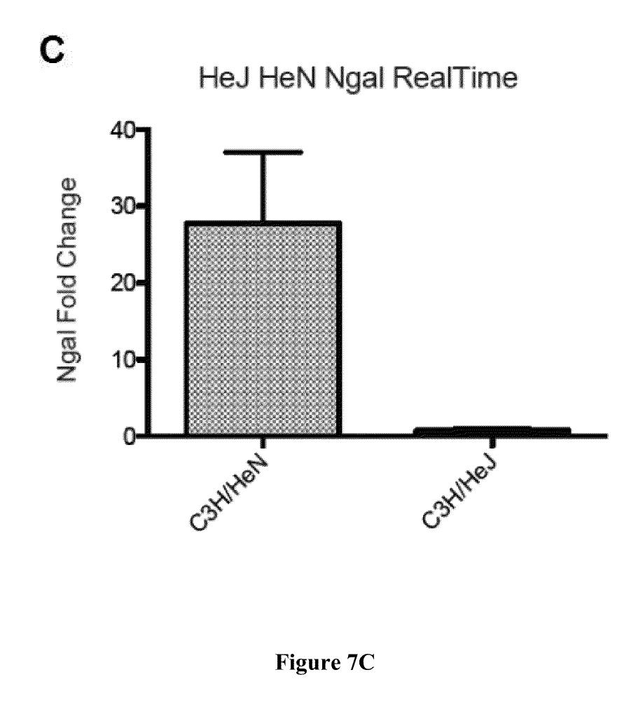

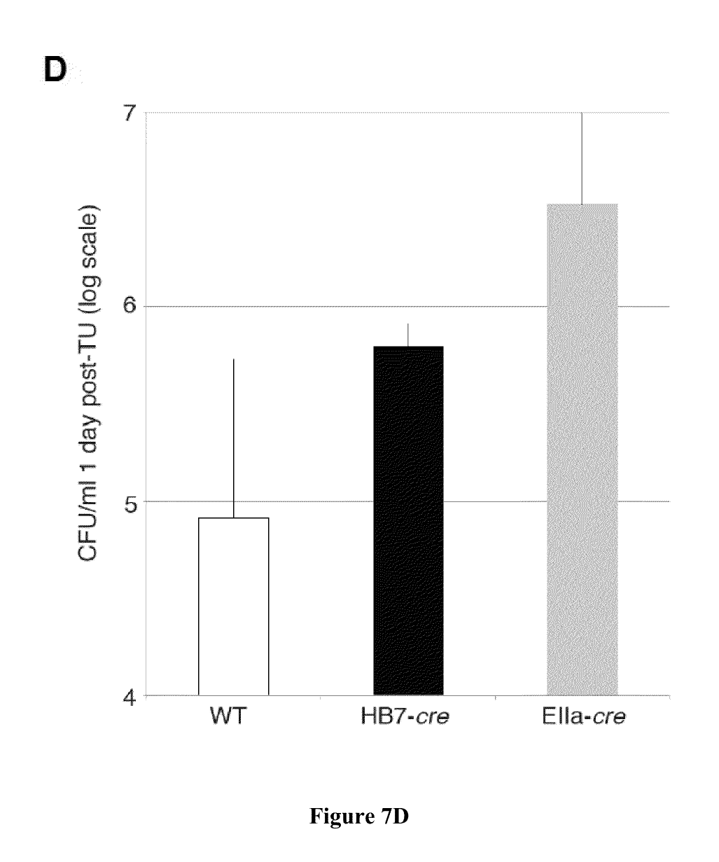

Many of the toll-like receptors are expressed in the kidney epithelium,²⁷ thus to determine which TLR is responsible for inducing NGAL expression in response to a UTI TLR2, TLR4 and TLR11 were challenged with TU injection of CFT073 (FIG. 7(c)). TLR2 and TLR4 are the most expressed in distal and proximal tubules and Bowman's capsule.²⁸ During sepsis and ischemia, both TLR2 and TLR4 are highly upregulated and their spatial distribution changes according to the event. Furthermore immunohistological localization reveals TLR2 and TLR4 in the apical membrane of the proximal tubule.²⁹ TLR11 has been shown to be expressed in the collecting duct and in the bladder epithelium and responds to UPECs.³⁰ However, a difference was observed in uCFU as early as a day after infection with the TLR11 mutant compared to its wild-type littermate (FIG. 7(c)). No differences in uCFUs from the TLR2 and TLR4 mutants were seen. A recent functional genomic study showed that C3H/HeJ bladders markedly expressed Ngal by gene arrays from laser capture microdissected urothelial cells.³¹ Furthermore, CFT073 has been observed to subvert TLR signaling via MyD88-dependent by secreting a TIR-domain protein that may bind directly to MyD88.³² Therefore, CFT073 may induce NGAL expression in the kidney through TLR11 via a MyD88-independent activation of NF-κβ.

The results described in this Example show that kidney epithelia produce NGAL, a bacteriostatic molecule, which can inhibit the growth of a highly pathogenic strain of uropathogenic bacteria (CFT073). NGAL is a secreted molecule shown to be a kidney growth factor,^{33,34} and has been shown to have a high affinity for iron bound catecholate-siderophores¹⁵ and endogenous iron bound catechol,³⁵ thus making it a potent antimicrobial by limiting Escherichia coli's¹⁴ access to iron. However it's role in urinary tract infections (UTI) was previously unknown. These data establish a rationale for the abundant NGAL secretion from the kidney in both aseptic and septic states in which the GU is part of the innate immune defense pathway and its expression is either prophylactic against a potential bacterial infection during an injury or protective against a current bacterial invasion. The results presented here show that NGAL is secreted in response to highly pathogenic strain of uropathogenic E. coli into the urinary space by the kidney epithelia⁹ and that signaling through TLR11 can inhibit bacterial growth.

These studies show that the kidney secretes NGAL in response to an impending bacterial infection. Perhaps the kidney is being primed for bacterial invasion due to the sharp reduction in glomerular filtration rate (urine flow). GFP reduction can occur from cast formation due to ischemia, toxic injury, and inflammation to the kidney while a reduction in urine flow can occur in injuries to the bladder such as obstruction and cancer. It is plausible that this reduction in urine flow from renal damage would make the kidney more vulnerable to bacterial invasion and thus pyelonephritis. Therefore, the kidney expresses a bacteriostatic molecule as a preemptive step to suppress an ascending UPEC from entering the kidney and subsequently entering the bloodstream.

Materials And Methods

Mouse Husbandry.

NgalloxP/loxP, NgalEII-Cre, NgalHoxB7-cre, C57BL6, C3H/HeJ, C3H/HeOuJ, and Tlr11 mice were raised and experimentally used in this study.

Ngal Cre-lox Targeting Construct Generation.

The BAC clone was made recombinogenic by transformation with a plasmid from the Red/ET cloning kit (Gene Bridges, Heidelberg, Germany) and the homology domains were subcloned into a backbone vector by a homologous recombination based Red/ET cloning method. A loxP site was inserted into intron 1, in a 2-step procedure. A loxP-flanked neo selection marker cassette (loxP-neo-loxP) is inserted by homologous recombination and then Cre is expressed in bacterial cells (EL350) to recombine the loxP sites and excise the selection marker, leaving a single loxP site. A neo cassette is inserted in a 2-step procedure into intron 5 using homologous recombinantion to insert a unique restriction site (Bsiw I) and then to ligate a neo cassette by conventional methods.

Electroporation into ES Cells.

The targeting vector was linearized, electroporated and clones selected with neo. Primers, A1, 2, 3 were 3′ of the short homology arm (SA) outside the region used to generate the targeting construct and N1 was located at the 5′ end of the Neo cassette amplify 2.3, 2.4, and 2.4 kb fragments respectively. Control PCR used T1 and T2, which are inside the targeting construct.

Excision of Neo Gene.

F0 mice derived from ES cells are crossed with a ubiquitous FLP deleter (including germ cells) under the control of human ACTB (βactin) promoter (B6; SJLTg(ACTFLPe)9205Dym/J (JAX® Mice Stock #003800).³⁶

The efficiency of FLP-excision of FRT-flanked DNA sequence was reported to 100% in F1 mice (heterozygote βactin-FLPe X heterozygote FRT-disrupted lacZ reporter gene driven by HMGCoA reductase promoter/enhancer sequence). Genotyping was performed in accordance with JAX® (the Jackson Laboratory) protocols. This strain was backcrossed to C57BL/6 for 3 generations and two more generations will be backcrossed also.

NGAL null F0 founder mice are crossed with the Cre deleter strain that expresses Cre at the one-cell stage of preimplantation embryo under the control of adenovirus EIIa promoter B6.FVB-Tg(EIIa-cre)C5379Lmgd/J (JAX® Mice Stock #003724).^{37,38}. The efficiency of Cre-mediated gene rearrangement is >50% in male mice homozygous for the chromosome carrying EIIa-cre transgene X female homozygous in the immunoglobulin light chain kappa locus for loxP-neo-loxP insertional cassette. 50% of F1 showed complete excision and the rest 50% showed partial excision of neo DNA. The complete excision was transmissible through the germ line. Genotyping was performed as per JAX® (the Jackson Laboratory) protocols. This is a congenic strain that has been backcrossed to C57BL/6 for at least 10 generations.

Neutrophil-Specific Cre.

There is an established Cre mouse strain which specifically expresses nuclear Cre in neutrophils and macrophages (B6.129P2-Lyzstml(cre)Ifo/J; JAX® Mice Stock #004781) under control of the endogenous Lysozyme M locus. This knock-in strategy for LysM-cre, rather than random transgene insertion, was especially important for this gene, since demethylation of 3′ enhancer downstream of LysM gene (exon 4) is involved in myeloid specific expression.³⁹ The Cre efficiency was nearly 100% in granulocytes and 83-93% in macrophages of F1 mice double transgenic for LysM-cre X lox1P-flanked beta-polymerase gene, and 75% in neutrophils and 82-91% in macrophages for HIF-1 and VEGF conditionally null mice. The excision of loxP-flanked DNA sequences in renal cells was not examined, but, at least, overall excision frequency was very low in the lung and spleen cells. Mouse lysozyme M gene is found only at low levels in the kidney, perhaps contaminating blood (0.4% of that in mature macrophage). Genotyping was performed in accordance with JAX® (the Jackson Laboratory) protocols. The strain has been backcrossed to C57BL/6 for more than 6 generations.

In Situ Hybridization.

NGAL RNA was detected using digoxigenin-labeled antisense riboprobes generated from cDNAs encoding NGAL (exon 1-7, 566 bp) by linearization with XhoI followed by T7 RNA polymerase. Kidneys were collected in ice-cold PBS and fixed overnight at 4° C. in 4% paraformaldehyde (PFA) in 0.1M phosphate buffer saline (PBS), briefly quenched in 50 mM NH₄Cl, cryoprotected overnight in 30% sucrose PBS and embedded and sectioned (16 μM) in Optimal Cutting Temperature (O.C.T.) compound. The sections were post-fixed in 4% paraformaldehyde (PFA) for 10 min, treated with proteinase K (1 mg/ml for 3 min), acetylated and prehybridized for 2 hrs, and then hybridized at 68-72° C. overnight. The prehybridization and hybridization solution was 50% formamide, 5′ SSC, 5′ Denhardts, 250 mg/ml baker's yeast RNA (Sigma), and 500 mg/ml herring sperm DNA (Sigma). Sections were washed at 72° C. in 5′ SSC for 5-10 minutes, then at 72° C. in 0.2° SSC for 1 hour and then stained overnight (4° C.) with an anti-digoxigenin antibody coupled with alkaline phosphatase (Boehringer-Mannheim), at a 1:5000 dilution in 0.1M Tris-HCl, pH 7.5, 0.15 M NaCl, 1% heat inactivated goat serum. Alkaline phosphatase activity was detected using BCIP, NBT (Boehringer-Mannheim) with 0.25 mg/ml levamisole in a humidified chamber for 1-3 days in the dark. Sections were dehydrated and mounted in Permount (Fisher Scientific).

Western Blot.

Urine and recombinant mouse NGAL protein standards were immunobloted using polyclonal anti-NGAL antibodies (R&D Systems, Minneapolis) and donkey anti-rabbit HRP-labelled IgG antibodies (Jackson Immunoresearch). NGAL protein was semi-quantified by comparison with standards using ImageJ software (NIH).

In situ hybridization and immunohistochemistry. Frozen and paraffin-embedded sections of mouse kidneys were prepared by following standard histological procedures. The paraffin sections were dewaxed and then rehydrated by using Histoclear (Fisher Scientific) and a gradient of ethanol, respectively, before in situ hybridization. A specific digoxigenin-labeled antisense riboprobes was generated from mouse Ngal cDNA (Genbank accession number: NM_{—}008491) by using a Dig-labelling kit (Roche Applied Biosystems), and was hybridized and detected as previously described.⁴⁰ The hybridized sections were counterstained with methyl green, dehydrated and mounted in Permount (Fisher Scientific). Frozen and paraffin-embedded sections were used for immunohistochemical analysis. Anti-mCherry (Clontech) and anti-v-ATPase B1/2 (Santa Cruz Biotechnology) were used at a 1:50 dilution and antigen was localized by HRP-DAB chromogen (R&D Systems) staining.

Real-Time PCR Analysis.

Total RNA was isolated with the mirVANA RNA extraction kit (Ambion), and the first strand cDNA was synthesized by using Superscript III (Invitrogen). Real-time PCR was performed to quantify Ngal mRNA expression in an iCycler MyiQ (Bio-Rad) with a SBR green supermix reagent (Bio-Rad) and Ngal-specific primers (Supplemental Table 1). β-actin was quantified as an internal control. The AACT method was used to calculated fold amplification of transcripts. Total RNA was isolated with the mirVANA RNA extraction kit (Ambion).

Real-Time PCR from C57BL6, Ngal−/−, Myd88−/−, Tlr2−/−, Tlr-4−/− and Tlr-4−/− was performed according to Bio-Rad SYBR GREEN, iCyclerMyiQ protocols. Target genes, including Ngal, β-actin, utilized respectively: Ngal 116 forward primer 5′-ctcagaacttgatccctgcc-3′ (SEQ ID NO.: 1) and NGALa593 reverse 5′-tccttgaggcccagacactt-3′ (SEQ ID NO.: 2); β-actin 415 forward primer 5′-ctaaggccaaccgtgaaaag-3′ (SEQ ID NO.: 3) and β-actin 696 reverse primer 5′-tctcagctgtggtggtgaag-3′(SEQ ID NO.: 4). The AACT method was used to calculated fold amplification of transcripts.

Mouse Urinary Tract Infection.

Female C57BL/6, NgalEII-Cre, NgalHoxB7-cre, C57B6, Tlr2−/−, Tlr-4−/−, and Tlr11−/− mice were used at an age of 8-16 weeks. In short, 10-20 μl of the bacterial suspension (5×10⁹ colony forming units/ml) was placed into the bladder of anesthesized mice through a soft polyethylene catheter. Bacterial tissue counts were obtained after homogenization of organ and serial plating on LB plates. Urinary colony forming units (CFU) were determined by direct collection of urine from the mouse and followed by plating.

Western Blot. NGAL was quantified by western blots, using non-reducing 4-15% tris-HCL gels (Bio-Rad, Laboratories, Inc. Hercules, Calif.) and monoclonal (1:1000; AntibodyShop, Gentofte, Denmark) or rabbit polyclonal antibodies (R&DSystems, Minneapolis) together with standards (0.2-10 ng) of human or mouse recombinant NGAL protein. NGAL was reproducibly detected to 0.4 ng/lane. NGAL expression was quantified using ImageJ software (NIMH).

### REFERENCES FOR EXAMPLE 1

1. Litwin, M. S., et al. Urologic diseases in America Project: analytical methods and principal findings. J Urol 173, 933-937 (2005).
2. Brzuszkiewicz, E., et al. How to become a uropathogen: comparative genomic analysis of extraintestinal pathogenic Escherichia coli strains. Proceedings of the National Academy of Sciences of the United States of America 103, 12879-12884 (2006).
3. McNeeley, S. G., Baselski, V. S. & Ryan, G. M. An evaluation of two rapid bacteriuria screening procedures. Obstet Gynecol 69, 550-553 (1987).
4. Pfaller, M. A. & Koontz, F. P. Laboratory evaluation of leukocyte esterase and nitrite tests for the detection of bacteriuria. J Clin Microbiol 21, 840-842 (1985).
5. Little, P., et al. Dipsticks and diagnostic algorithms in urinary tract infection: development and validation, randomised trial, economic analysis, observational cohort and qualitative study. Health Technol Assess 13, iii-iv, ix-xi, 1-73 (2009).
6. Gorelick, M. H. & Shaw, K. N. Screening tests for urinary tract infection in children: A meta-analysis. Pediatrics 104, e54 (1999).
7. Nickolas, T. L., et al. Sensitivity and specificity of a single emergency department measurement of urinary neutrophil gelatinase-associated lipocalin for diagnosing acute kidney injury. Ann Intern Med 148, 810-819 (2008).
8. Bennett, M., et al. Urine NGAL predicts severity of acute kidney injury after cardiac surgery: a prospective study. Clin J Am Soc Nephrol 3, 665-673 (2008).
9. Paragas, N., Qiu, A., and Barasch, J. Seeing Kidney Disease in Real-time: NGAL Reporter Mouse Detects the Response of Tubular Segments to Cell Stressors In vivo (In Press). Nature medicine (2010).
10. Mishra, J., et al. Identification of neutrophil gelatinase-associated lipocalin as a novel early urinary biomarker for ischemic renal injury. J Am Soc Nephrol 14, 2534-2543 (2003).
11. Mori, K., et al. Endocytic delivery of lipocalin-siderophore-iron complex rescues the kidney from ischemia-reperfusion injury. J Clin Invest 115, 610-621 (2005).
12. Mishra, J., et al. Amelioration of ischemic acute renal injury by neutrophil gelatinase-associated lipocalin. J Am Soc Nephrol 15, 3073-3082 (2004).
13. Barasch, J. & Mori, K. Cell biology: iron thievery. Nature 432, 811-813 (2004).
14. Flo, T. H., et al. Lipocalin 2 mediates an innate immune response to bacterial infection by sequestrating iron. Nature 432, 917-921 (2004).
15. Goetz, D. H., et al. The neutrophil lipocalin NGAL is a bacteriostatic agent that interferes with siderophore-mediated iron acquisition. Mol Cell 10, 1033-1043 (2002).
16. Lakso, M., et al. Efficient in vivo manipulation of mouse genomic sequences at the zygote stage. Proceedings of the National Academy of Sciences of the United States of America 93, 5860-5865 (1996).
17. Zhu, X. D. & Sadowski, P. D. Cleavage-dependent ligation by the FLP recombinase. Characterization of a mutant FLP protein with an alteration in a catalytic amino acid. The Journal of biological chemistry 270, 23044-23054 (1995).
18. Sauer, B. Functional expression of the cre-lox site-specific recombination system in the yeast Saccharomyces cerevisiae. Molecular and cellular biology 7, 2087-2096 (1987).
19. Yu, J., Carroll, T. J. & McMahon, A. P. Sonic hedgehog regulates proliferation and differentiation of mesenchymal cells in the mouse metanephric kidney. Development (Cambridge, England) 129, 5301-5312 (2002).
20. Bulut, Y., et al. Chlamydial heat shock protein 60 activates macrophages and endothelial cells through Toll-like receptor 4 and MD2 in a MyD88-dependent pathway. J Immunol 168, 1435-1440 (2002).
21. Ohashi, K., Burkart, V., Flohe, S. & Kolb, H. Cutting edge: heat shock protein 60 is a putative endogenous ligand of the toll-like receptor-4 complex. J Immunol 164, 558-561 (2000).
22. Vabulas, R. M., et al. HSP70 as endogenous stimulus of the Toll/interleukin-1 receptor signal pathway. J Biol Chem 277, 15107-15112 (2002).
23. Okamura, Y., et al. The extra domain A of fibronectin activates Toll-like receptor 4. J Biol Chem 276, 10229-10233 (2001).
24. Termeer, C., et al. Oligosaccharides of Hyaluronan activate dendritic cells via toll-like receptor 4. The Journal of experimental medicine 195, 99-111 (2002).
25. Johnson, G. B., Brunn, G. J., Kodaira, Y. & Platt, J. L. Receptor-mediated monitoring of tissue well-being via detection of soluble heparan sulfate by Toll-like receptor 4. J Immunol 168, 5233-5239 (2002).
26. Smiley, S. T., King, J. A. & Hancock, W. W. Fibrinogen stimulates macrophage chemokine secretion through toll-like receptor 4. J Immunol 167, 2887-2894 (2001).
27. El-Achkar, T. M. & Dagher, P. C. Renal Toll-like receptors: recent advances and implications for disease. Nat Clin Pract Nephrol 2, 568-581 (2006).
28. Wolfs, T. G., et al. In vivo expression of Toll-like receptor 2 and 4 by renal epithelial cells: IFN-gamma and TNF-alpha mediated up-regulation during inflammation. J Immuno1168, 1286-1293 (2002).
29. Kim, B. S., et al. Ischemia-reperfusion injury activates innate immunity in rat kidneys. Transplantation 79, 1370-1377 (2005).
30. Zhang, D., et al. A toll-like receptor that prevents infection by uropathogenic bacteria. Science 303, 1522-1526 (2004).
31. Reigstad, C. S., Hultgren, S. J. & Gordon, J. I. Functional genomic studies of uropathogenic Escherichia coli and host urothelial cells when intracellular bacterial communities are assembled. The Journal of biological chemistry 282, 21259-21267 (2007).
32. Cirl, C., et al. Subversion of Toll-like receptor signaling by a unique family of bacterial Toll/interleukin-1 receptor domain-containing proteins. Nature medicine 14, 399-406 (2008).
33. Yang, J., et al. An iron delivery pathway mediated by a lipocalin. Mol Cell 10, 1045-1056 (2002).
34. Yang, J., Mori, K., L1, J. Y. & Barasch, J. Iron, lipocalin, and kidney epithelia. Am J Physiol Renal Physiol 285, F9-18 (2003).
35. Bao, G., Clifton, M., Hoette, T., Mori, K., Deng, S., Qiu, A., Viltard, M., Williams, D, Paragas, N, Leete, T, Kulkarni, R., L1, X., Lee, B., Kalandadze, A., Ratner, A., Pizarro, J., Schmidt-Ott, K., Landry, D., Raymond, K., Strong, R. and Barasch, J. Iron Traffics in Circulation Bound to Siderocalin (Ngal) Complexed with a Bacterial Metabolite Called Catechol (In Press). Nat Chem Bio (2010).
36. Rodriguez, C. I., et al. High-efficiency deleter mice show that FLPe is an alternative to Cre-loxP. Nat Genet. 25, 139-140 (2000).
37. Dooley, T. P., Miranda, M., Jones, N. C. & DePamphilis, M. L. Transactivation of the adenovirus EIIa promoter in the absence of adenovirus E1A protein is restricted to mouse oocytes and preimplantation embryos. Development 107, 945-956 (1989).
38. Lakso, M., et al. Efficient in vivo manipulation of mouse genomic sequences at the zygote stage. Proc Natl Acad Sci USA 93, 5860-5865 (1996).
39. Clausen, B. E., Burkhardt, C., Reith, W., Renkawitz, R. & Forster, I. Conditional gene targeting in macrophages and granulocytes using LysMcre mice. Transgenic Res 8, 265-277 (1999).
40. L1, J. Y., et al. Scara5 is a ferritin receptor mediating non-transferrin iron delivery. Developmental cell 16, 35-46 (2009).

### Example 2

The numbers in superscript below refer to the corresponding numbered reference(s) at the end of this Example.

The results presented herein, and in Example 1, demonstrate that specific segments of the kidney epithelia rapidly produce NGAL (siderocalin), which has bacteriostatic activity, blocking growth of uropathogenic bacteria located in the urinary tract, including the bladder. Thus, the kidney plays a role in innate defense. NGAL inhibits the growth of bacteria by capturing at high affinity iron bound to catecholate-siderophores and/or endogenous catechols', making it a potent antimicrobial by limiting E. coli's access to iron. The kidney and bladder detected a urinary tract infection (UTI) in part by segmentally localized expression of Toll-like receptors (TLRs), which trigger NGAL expression. This data establishes a rationale for the abundant NGAL secretion from the kidney in both septic and perhaps in aseptic states, demonstrating that the kidney defends the urinary system via the exocrine delivery of NGAL.

In Humans^{4,5} and mice⁶ secreted lipocalin Neutrophil Gelantinase Associated Lipocalin (NGAL) is markedly upregulated and expressed by the kidney in proportion to the dose of injurious stimuli such as ischemia-reperfusion (I/R)⁶, hypoxia, drug toxicity⁶, and bacterial infection.^{4,6-9} Previously, it was not clear why this protein is expressed in the urinary system after kidney damage of different types. NGAL is a bacteriostatic protein.¹⁰ It binds catecholate-siderophore¹¹ which sequesters iron from bacteria.

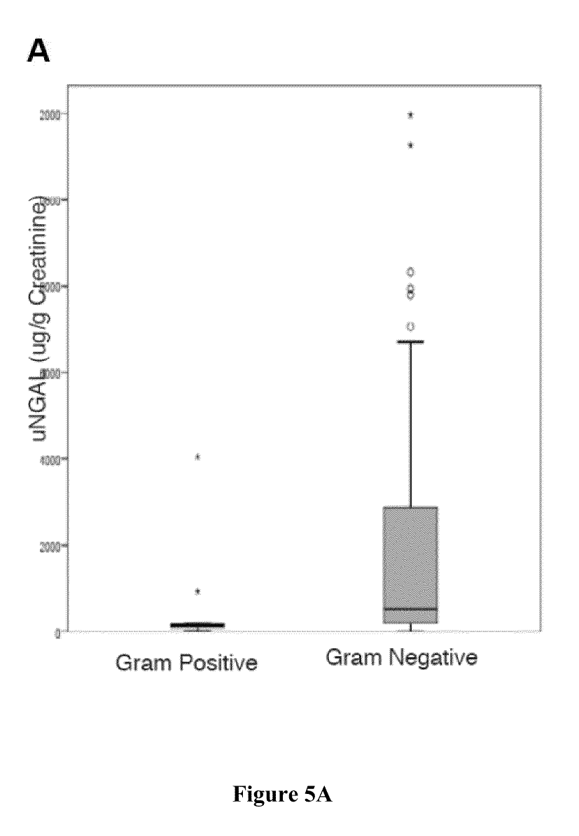

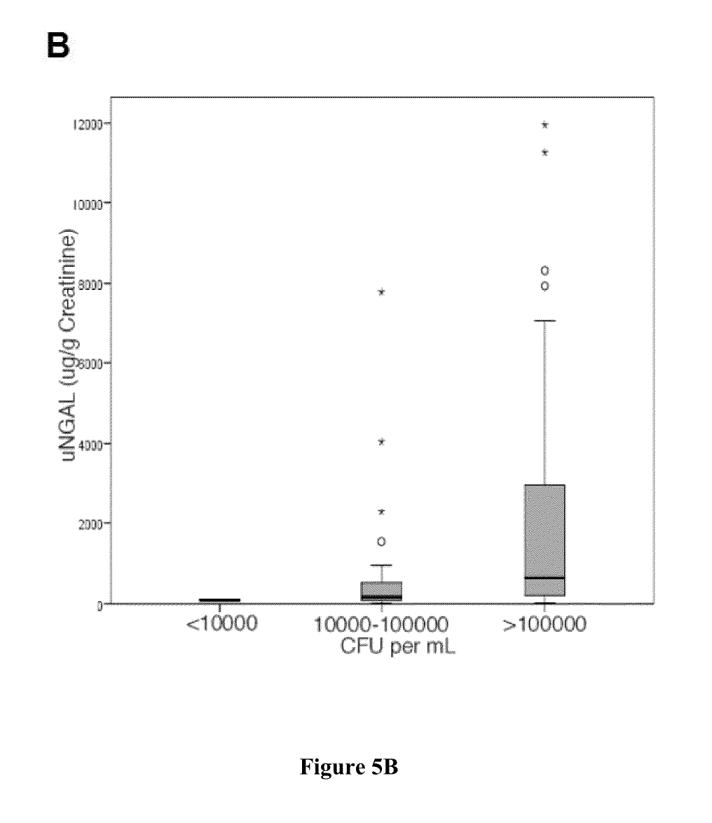

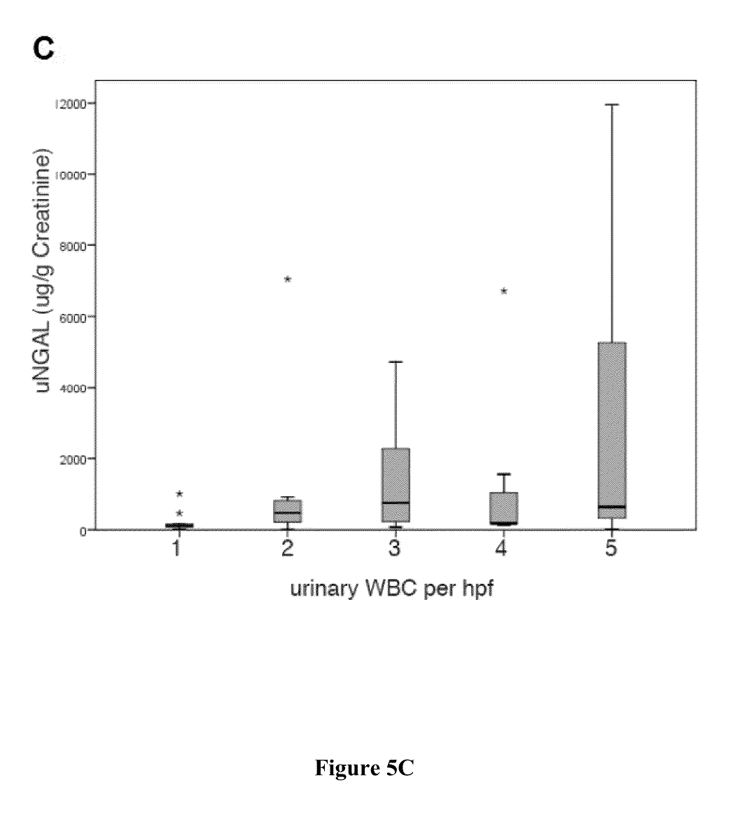

In a large multi-center study it was discovered that patients with UTI caused by gram negative bacteria (n=77) had significantly elevated uNGAL compared to patients with UTI due to gram positive bacteria (n=10) (FIG. 5a, 2078±3215 vs 592±1242 mg/g creatinine, P=0.01). A dose-response relationship was seen between number of colony forming units (CFU) of UTI-causing bacteria and uNGAL levels (FIG. 5b). Patients with more than 105 CFU (n=64) had significantly more uNGAL compared to patients with between 104 and 105 CFU (n=21) (2251±3353 mg/g creatinine, 928±1850 mg/gm creatinine, P=0.02). Consistently, the number of white blood cells in the urine and uNGAL levels were also directly proportional to the amount of uNGAL. Patients with either 11-20 cells per high powered field (hpf) (n=16) or <30 cells per hpf (n=46) had significantly elevated uNGAL levels compared to patients with between 3 to 5 cells per hpf (n=10) (1455±1534 μg/g creatinine in 11-20 cells/hpf, 3023±4067 μg/g creatinine <30 cells/hpf versus 219±309 μg/g creatinine for 3-5 cells/hpf, P=0.002 and <0.001) (FIG. 5c).

To establish the relationship between NGAL and UPEC (uropathogenic Escherichia coli, we grew a pyelonephritogenic clinical UPEC isolate (CFT073) in M9 minimal medium supplemented with MgCl₂ and glucose (green hashed line: FIG. 1a), and monitored bacterial growth by spectrophotometry. Bacterial growth was significantly inhibited (FIG. 1a) when the UPEC was grown with the addition of mouse (m) NGAL (red line and open red squares: 5 μM), but it could be rescued by oversaturating NGAL with the addition of FeCl₃ (blue line with open blue circles: 1 mM). This data is consistent with NGAL's activity in iron scavenging.¹¹

To determine the role NGAL has in an acute UTI in vivo a conditional NgalloxP/loxP animal was generated and used to generate tissue specific knockouts (Methods and). First, a NgalloxP/loxP mouse was bred with an EIIa-Cre12 mouse (NgalloxP/loxP, EIIa-Cre) which generated a knock-out of NGAL in all cells. Ngal wild-type (Ngal+/+) mice (white bar) and NgalloxP/loxP, EIIa-Cre (black bars) mice were challenged with a transurethral (TU) infection of the UPEC (10-20 ul of 5×108 CFU/ml CFT073) and urinary (u) NGAL and urinary colony forming units (c.f.u.) were monitored for one week (FIG. 1b). In Ngal+/+ mice we noted that the secretion of uNGAL mirrored both the onset of the urinary tract infection (UTI), indicated by log order increases in uCFU, and the resolution of the acute phase of the infection (3-4 d, FIG. 1b). uNGAL was detected within 24 h of the application of the UPEC to the bladder. Ngal+/+ mice clear the acute UTI within 3-5 d after the initial challenge of the UPEC, but animals without a functioning Ngal allele took significantly more time (6 days) than the wild type mice to clear the UPEC (NgalloxP/loxP, EIIa-Cre mice had significantly more bacteria than Ngal+/+ at 2, 3, 4 and 5 d post-TU, P<0.05, UTest: FIG. 1b). Hence, uNGAL is necessary to rapidly clear an acute UTI by an enterochelin dependent UPEC and importantly that the protein itself is sufficient for bacteriostasis.

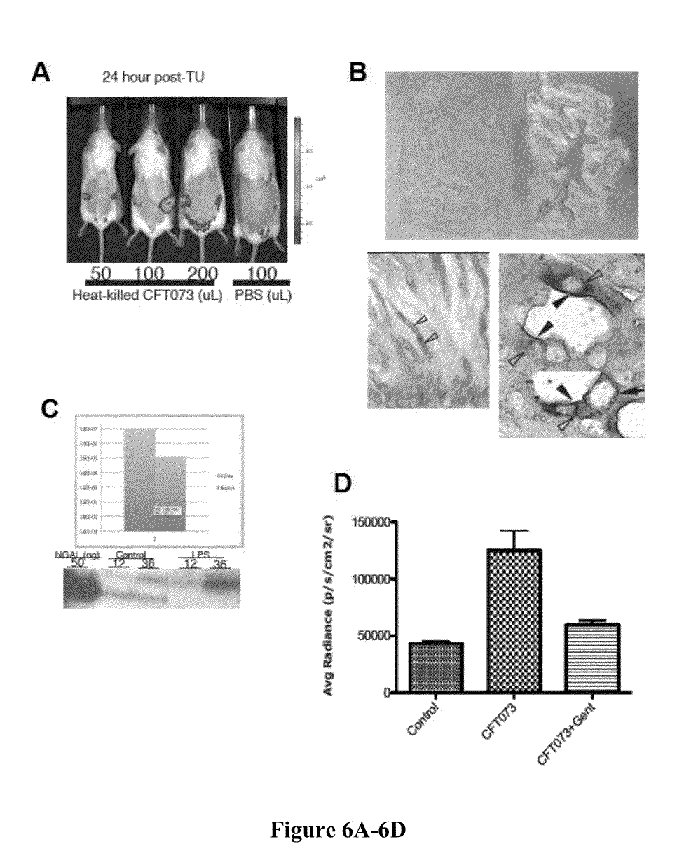

A striking feature of the GU infection was the distant response of the kidney to an acute bladder event. To evaluate this TU injection was performed using heat-killed CFT073 (108 CFU/ml) into the mouse bladder of the NGAL bioluminescent reporter animal.⁶ TU volumes ranging from 50-200 uL of bacterial detritus activated NGAL-luc2/mC expression in the bladder, ureter and the kidney. Quantitative analysis of NGAL-luc2/mC signal from the kidney revealed a 2.2 fold increase in NGAL-luc2/mC expression compared to PBS control (FIG. 6a). To determine the relative contribution of these organs to uNGAL QPCR was performed and the copy number of Ngal per organ 1 d post-TU challenge with the UPEC was quantified. Intraperitoneal (IP) injection was used as a control to stimulate Ngal expression. With either systemic or gastrourogenital application of bacteria, the kidney was the major contributor to uNGAL as measured by the number of copies of NGAL respective of total RNA (FIG. 6b). Furthermore a two fold increase of Ngal expression was observed without the presence of bacteria in the organ. To determine the cellular source of uNGAL in response an acute UPEC infection, in situ hybridization was performed in Ngal+/+ kidneys 1 d post-TU injection of the UPEC (5×109 CFU/ml). High levels of Ngal RNA were expressed by bladder epithelium (FIG. 6c) and through microscopic examination of the kidney tissue, Ngal mRNA was identified in epithelia of the Thick Ascending Limb of Henle (TAL) and the Collecting Ducts (CD)(FIG. 6d). Co-staining of the Ngal positive GU epithelial cells with anti-LPS antibody revealed that cells directly in contact with the UPEC were expressing Ngal. UPEC adherence to distal epithelial cells has was previously observed by Chassin et al to be specific to intercalated cells (ICs) of the collecting duct (CD).¹³ In the present experiments it was observed that a subset of these ICs, alpha-IC (A-IC), specifically recognize the bacteria and express Ngal (FIG. 6c). To evaluate whether the effect on the CD epithelia by bacteria may be a direct interaction, primary kidney cells isolated from Ngal-Luc2/mC reporter mice⁶ were used. As shown in FIG. 6a, NGAL-Luc2 expression in kidney cells was markedly upregulated (FIG. 6d) over 24 hours after an initial innoculum of bacteria. Thus, many types of GU epithelium generate Ngal RNA and the urogenital system plays an essential role in limiting the growth of UPECs via expression of the urinary bacteriostatic molecule NGAL.

Because the data showed that both the kidney and the bladder might contribute to the uNGAL pool, Ngal expression was further studied by selectively knocking out Ngal in the NGAL expressing segments of the kidney.⁶ We deleted Ngal in the collecting duct (CD) by generating a NgalloxP/loxP, HoxB7/cre14 (NgalloxP/loxP, HoxB7/cre) CKO. HoxB7/cre has been noted to be expressed in the ureteric epithelium of the distal, non-branching medullary collecting ducts and continues into the epithelium of the ureter.¹⁴ We found that the HoxB7 compartment was a major contributor to uNGAL because uNGAL protein was decreased several-fold. Moreover there was greater than a log order increase in median uCFU after a TU challenge with the UPEC compared to the wild type mice (FIG. 7a). These data support findings that Ngal expression was localized after urinary infection was localized in the collecting duct.⁶ To examine whether the bladder epithelia also contributed to uNGAL, we incubated explanted bladders from mice with UTIs and collected conditioned media. Although we detected NGAL, it was only a fraction seen in the urine over the same period (not shown). Therefore many types of GU epithelia generate uNGAL protein, but the kidney is the major contributor during a UTI. A combination of in situ hybridization, organ copy number, and segment specific knockouts, indicated in vivo that the major source of Ngal RNA is the kidney and the major source of NGAL protein is the kidney.

Previous findings⁶ and data from isolated primary cells in FIG. 1d, revealed that bacterial gram-negative components such as lipid A bind to TLRs, such as TLR4, and activate NF-κB⁸ which induce Ngal in vitro. To determine whether uNGAL originated from the kidney, we performed kidney transplantation between Tlr-4-mutant CH3/HeJ mice and wild type CH3/HeOuJ mice. TLRs are receptors for bacterial infection. TLRs^{2,4,5,11} have been assumed to be expressed in the GU.¹⁵ To localize these receptors in situ hybridization was performed to map which segments were capable of signaling via which toll-like receptor. To evaluate the contribution of TLR signaling to NGAL expression, a cross-transplant model was utilized using TLR mutants and systemic administration of LPS as a positive control. The CH3/HeJ kidneys from the LPS-insensitive mice were transplanted into CH3/HeOuJ LPS-sensitive (control) mouse bodies, and vice versa. Two weeks after graft maturation, when uNGAL and sCr had stabilized to normal values, a low dose of lipid A (1 mg/kg of body weight) was administered to induce Ngal expression in the kidney (FIG. 7b). QPCR revealed that the wild type kidney in the Tlr-4-mutant body had a 15.6±2.3 fold increase in Ngal expression while the Tlr-4-mutant kidney in the wild type body had only a 4.5±2.6 fold increase in Ngal expression. It is not surprising that there was some Ngal induction in the knockout kidney because CH3/HeJ are partial KOs, and it has also been previously shown that lipid A can signal through the MyD88-dependent pathway without a functioning Tlr4ref. These results suggests that TLR4 is the receptor to lipid A in the kidney, and indicate that NGAL is induced by LPS activation of the TLR4::NfKB pathway.

To examine the roles of TLR's in the expression of NGAL in a UTI, TU experiments were performed on C3H/OuJ and C3H HeJ TLR4 mutants to establish the signaling pathway for uNGAL expression during an acute urinary tract infection. uNGAL expression and uCFU was measured.

The results of the study described in this Example, and those described in Example 1, demonstrate that NGAL expression is stimulated by activation TLRs located in different segments of the urogenital tract, and that UTI activates NGAL in different segments. Thus, the kidney is an exocrine organ that senses the presence of UPECs via Toll-like receptors and secretes uNGAL into the urinary space to suppress the infection.

### Methods

Mouse husbandry. NgalloxP/loxP, NgalEII-Cre, NgalHoxB7-cre, C57BL6, C3H/HeJ, C3H/HeOuJ, C3H/HeN, Tlr2, Tlr4, Tlr5, Tlr11, MyD88, Ticam1, and Ngal-Luc2/mC mice were raised and used in this study.

Ngal Cre-lox Targeting Construct Generation. The BAC clone was made recombinogenic by transformation with a plasmid from the Red/ET cloning kit (Gene Bridges, Heidelberg, Germany) and the homology domains were subcloned into a backbone vector by homologous recombination based Red/ET cloning method. A loxP site was inserted into intron 1, in a 2-step procedure. A loxP-flanked neo selection marker cassette (loxP-neo-loxP) is inserted by homologous recombination and then Cre is expressed in bacterial cells (EL350) to recombine the loxP sites and excise the selection marker, leaving a single loxP site. A neo cassette is inserted in a 2-step procedure into intron 5 using homologous recombinantion to insert a unique restriction site (Bsiw I) and then to ligate a neo cassette by conventional methods.

Electroporation into ES Cells.

The targeting vector was linearized, electroporated and clones selected with neo. Primers, A1, 2, 3 were 3′ of the short homology arm (SA) outside the region used to generate the targeting construct and N1 was located at the 5′ end of the Neo cassette amplify 2.3, 2.4, and 2.4 kb fragments respectively. Control PCR used T1 and T2, which are inside the targeting construct.

Excision of neo gene F0 mice derived from ES cells are crossed with a ubiquitous FLP deleter (including germ cells) under the control of human ACTB (βactin) promoter (B6; SJLTg(ACTFLPe)9205Dym/J (JAX® Mice Stock #003800)16.

The efficiency of FLP-excision of FRT-flanked DNA sequence was reported to 100% in F1 mice (heterozygote βactin-FLPe X heterozygote FRT-disrupted lacZ reporter gene driven by HMGCoA reductase promoter/enhancer sequence). Genotyping was performed in accordance with JAX® (the Jackson Laboratory) protocols. This strain was backcrossed to C57BL/6 for 3 generations.

Ngal null F0 founder mice were crossed with the Cre deleter strain that expresses Cre at the one-cell stage of preimplantation embryo under the control of adenovirus EIIa promoter B6.FVB-Tg(EIIa-cre)C5379Lmgd/J (JAX® Mice Stock #003724).12,17 The efficiency of Cre-mediated gene rearrangement >50% in male mice homozygous for the chromosome carrying EIIa-cre transgene X female homozygous in the immunoglobulin light chain kappa locus for loxP-neo-loxP insertional cassette. 50% of F1 showed complete excision and the rest 50% showed partial excision of neo DNA. The complete excision was transmissible through the germ line. Genotyping was performed in accordance with JAX® (the Jackson Laboratory) protocols. This is a congenic strain that has been backcrossed to C57BL/6 for at least 10 generations.

Neutrophil-specific Cre. There is an established Cre mouse strain which specifically expresses nuclear Cre in neutrophils and macrophages (B6.129P2-Lyzstml(cre)Ifo/J; JAX® Mice Stock #004781) under control of the endogenous Lysozyme M locus. This knock-in strategy for LysM-cre, rather than random transgene insertion, was especially important for this gene, since demethylation of 3′ enhancer downstream of LysM gene (exon 4) is involved in myeoild specific expression.¹⁸ The Cre efficiency was nearly 100% in granulocytes and 83-93% in macrophages of F1 mice double transgenic for LysM-cre X lox1P-flanked beta-polymerase gene, and 75% in neutrophils and 82-91% in macrophages for HIF-1 and VEGF conditionally null mice. The excision of loxP-flanked DNA sequences in renal cells was not examined, but, at least, overall excision frequency was very low in the lung and spleen cells. Mouse lysozyme M gene is found only at low levels in the kidney, perhaps contaminating blood (0.4% of that in mature macrophage). Genotyping was performed in accordance with JAX® (the Jackson Laboratory) protocols. The strain has been backcrossed to C57BL/6 for more than 6 generations.

In Situ Hybridization.

NGAL RNA was detected using digoxigenin-labeled antisense riboprobes generated from cDNAs encoding Ngal (exon 1-7, 566 bp) by linearization with XhoI followed by T7 RNA polymerase. Kidneys were collected in ice-cold PBS and fixed overnight at 4° C. in 4% paraformaldehyde (PFA) in 0.1M phosphate buffer saline (PBS), briefly quenched in 50 mM NH4Cl, cryoprotected overnight in 30% sucrose PBS and embedded and sectioned (16 μM) in Optimal Cutting Temperature (O.C.T.) compound. The sections were post-fixed in 4% PFA for 10 min, treated with proteinase K (1 mg/ml for 3 min), acetylated and prehybridized for 2 hrs, and then hybridized at 68-72° C. overnight. The prehybridization and hybridization solution was 50% formamide, 5′ SSC, 5′ Denhardts, 250 mg/ml baker's yeast RNA (Sigma), and 500 mg/ml herring sperm DNA (Sigma). Sections were washed at 72° C. in 5′ SSC for 5-10 minutes, then at 72° C. in 0.2° SSC for 1 hour and then stained overnight (4° C.) with an anti-digoxigenin antibody coupled with alkaline phosphatase (Boehringer-Mannheim), at a 1:5000 dilution in 0.1M Tris-HCl, pH 7.5, 0.15 M NaCl, 1% heat inactivated goat serum. Alkaline phosphatase activity was detected using BCIP, NBT (Boehringer-Mannheim) with 0.25 mg/ml levamisole in a humidified chamber for 1-3 days in the dark. Sections were dehydrated and mounted in Permount (Fisher Scientific).

Bioluminescence and Fluorescence Imaging of Living Ngal-Luc2/mC Reporter Mice.

Ngal-Luc2/mC reporter mice were injected ip with 150 mg/kg of D-luciferin (Caliper Life Sciences) in PBS (pH 7.0). Ten minutes later, the mice are anesthesized (2.5% isofluorane) and a whole body image was acquired for 30s using the Xenogen IVIS optical imaging system (Xenogen Corp., Almeda, Calif.) with an open excitation filter and an open emission filter for luminescence and fluorescence, respectively. Regions of interest (ROIs) were drawn on the dorsal side of the animal and quantified by using Living Image Software version 3.119. Counts in the ROIs were detected by a CCD camera digitizer and were converted to physical units of radiance in photons/s/cm2/steradian¹⁹.

Western Blot. Urine and recombinant mouse NGAL protein standards were immunoblotted using polyclonal anti-NGAL antibodies (R&D Systems, Minneapolis) and donkey anti-rabbit HRP-labelled IgG antibodies (Jackson Immunoresearch). NGAL protein was semi-quantified by comparison with standards using ImageJ software (NIH).

In Situ Hybridization and Immunohistochemistry.

Frozen and paraffin-embedded sections of mouse kidneys were prepared by following standard histological procedures. The paraffin sections were dewaxed and then rehydrated by using Histoclear (Fisher Scientific) and a gradient of ethanol, respectively, before in situ hybridization. A specific digoxigenin-labeled antisense riboprobes was generated from mouse Ngal cDNA (Genbank accession number: NM_{—}008491) by using a Dig-labelling kit (Roche Applied Biosystems), and was hybridized and detected as previously described²⁰. The hybridized sections were counterstained with methyl green, dehydrated and mounted in Permount (Fisher Scientific). Frozen and paraffin-embedded sections were used for immunohistochemical analysis. Anti-mCherry (Clontech) and anti-v-ATPase B1/2 (Santa Cruz Biotechnology) were used at a 1:50 dilution and antigen was localized by HRP-DAB chromogen (R&D Systems) staining.

Real-Time PCR Analysis.

Total RNA was isolated with the mirVANA RNA extraction kit (Ambion), and the first strand cDNA was synthesized by using Superscript III (Invitrogen). Real-time PCR was performed to quantify Ngal mRNA expression in an iCycler MyiQ (Bio-Rad) with a SBR green supermix reagent (Bio-Rad) and Ngal-specific primers. β-actin was quantified as an internal control. The AACT method was used to calculated fold amplification of transcripts.

Isolation and culture of primary cells. Whole kidneys were dissected from perfused Luc2/mC di-fusion reporter mice (8-12 weeks of age) and kidney cells dispersed by collagenase (2 mg/ml; Sigma), followed by culture (1×10⁵/well in 24-well plates; Falcon) in DMEM/F12 medium supplemented with 10% FBS, 1% penicillin-streptomycin, and 46 mg/l L-Valine for 24 hours.

Primary cells were treated for 24 hours with 10⁴ CFU/ml uropathogenic E. coli (CFT073) and in some cases with 100 μg/ml gentamicin. Alternatively, primary cells were treated with Lipid A and in some cases with NF-kB inhibitors, MG132 (Cayman Chemical), and Analogues 27, 30, and 3121 and Analogue 3022. The Luciferase substrate (Dual-Glo™ Luciferase Assay System; Promega) was added and luminescence from Luc2 and fluorescence from mC (excitation of 500-550 nm and emission of 575-650 nm) were imaged in a Xenogen IVIS optical imaging system.

Total RNA was isolated with the mirVANA RNA extraction kit (Ambion).

Real-Time PCR from C57BL6, Ngal−/−, Myd88−/−, C3H/HeJ, CeH/HeOuJ, Tlr2−/−, Tlr-4−/− and Tlr11−/− was performed according to Bio-Rad SYBR GREEN, iCyclerMyiQ protocols. Target genes, including Ngal, β-actin, utilized respectively: Ngal 116 forward primer 5′-ctcagaacttgatccctgcc-3′ SEQ ID NO.: 1) and NGALa593 reverse 5′-tccttgaggcccagacactt-3′ (SEQ ID NO.: 2); β-actin415 forward primer 5′-ctaaggccaaccgtgaaaag-3′(SEQ ID NO.: 3) and β-actin 696 reverse primer 5′-tctcagctgtggtggtgaag-3′ ((SEQ ID NO.: 4). The AACT method was used to calculated fold amplification of transcripts.

Mouse Urinary Tract Infection.

We used female C57BL/6, NgalEII-Cre, NgalHoxB7-cre, MyD88−/−, C57B6, Trif, C3H/HeJ, CeH/HeOuJ, Tlr2−/−, Tlr-4−/−, and Tlr11−/− mice at an age of 8-16 weeks. In short, we placed 10-20 μl of the bacterial suspension (5×10⁹ colony forming units/ml) into the bladder of anesthesized mice through a soft polyethylene catheter. We obtained bacterial tissue counts after homogenization of organ and serial plating on LB plates. Urinary colony forming units (CFU) were determined by direct collection of urine from the mouse and followed by plating.

Human Data.

This is a cross sectional analysis derived from a study of the utility of uNGAL to discriminate patients with acute kidney injury who presented to an emergency department at three different hospital sites. All patients presenting to the ED at the three different sites who were admitted to the hospital were approached for participation in this study. A total of 2457 patients were enrolled. Patients were consented, and a sample of their urine collected. The urine was centrifuged for 10 minutes at 12,000 rpm, the supernatant collected and frozen at −80° C. Patients who were less than 18 years of age, in end-stage renal disease, had a hospital stay less than 24 hours, or already on hemodialysis were excluded. The data presented here is a cross-sectional analysis of this data to investigate relationships between uNGAL and ascending infections of the urinary tract. Patients were assigned to a group based on urinary studies and culture results done in the emergency department. Patients in this analysis were identified as having UTI, which is defined as positive urine culture of a non-contaminate organism. Patients with a UTI secondary to urinary tract obstruction were excluded as this has been shown to elevate uNGAL levels independently of UTI status (unpublished data).

SPSS version 16.0 was used for all human data analysis (SPSS, Chicago, Ill.). All continuous data were log-transformed prior to analysis and presented as non-log-transformed values. T-test for unequal variances was used for comparisons.

Stressors.

Lipid A was obtained from Alexis Biochemical.

Western Blot.

NGAL was quantified by western blots, using non-reducing 4-15% tris-HCL gels (Bio-Rad, Laboratories, Inc. Hercules, Calif.) and monoclonal (1:1000; AntibodyShop, Gentofte, Denmark) or rabbit polyclonal antibodies (R&D Systems, Minneapolis) together with standards (0.2-10 ng) of human or mouse recombinant NGAL protein. NGAL was reproducibly detected to 0.4 ng/lane.

### REFERENCES FOR EXAMPLE 2

1. Bao, G., Clifton, M., Hoette, T., Mori, K., Deng, S., Qiu, A., Viltard, M., Williams, D, Paragas, N, Leete, T, Kulkarni, R., Li, X., Lee, B., Kalandadze, A., Ratner, A., Pizarro, J., Schmidt-Ott, K., Landry, D., Raymond, K., Strong, R. and Barasch, J. Iron traffics in circulation bound to a siderocalin (Ngal)-catechol complex. Nat Chem Biol 6, 7 (2010).
2. Litwin, M. S., et al. Urologic diseases in America Project: analytical methods and principal findings. J Urol 173, 933-937 (2005).
3. Brzuszkiewicz, E., et al. How to become a uropathogen: comparative genomic analysis of extraintestinal pathogenic Escherichia coli strains. P Natl Acad Sci USA 103, 12879-12884 (2006).
4. Mori, K., et al. Endocytic delivery of lipocalin-siderophore-iron complex rescues the kidney from ischemia-reperfusion injury. J Clin Invest 115, 610-621 (2005).
5. Nickolas, T. L., et al. Sensitivity and specificity of a single emergency department measurement of urinary neutrophil gelatinase-associated lipocalin for diagnosing acute kidney injury. Ann Intern Med 148, 810-819 (2008).
6. Paragas, N., et al. The Ngal reporter mouse detects the response of the kidney to injury in real time. Nature medicine 17, 216-222 (2011).
7. Mishra, J., et al. Amelioration of ischemic acute renal injury by neutrophil gelatinase-associated lipocalin. J Am Soc Nephrol 15, 3073-3082 (2004).
8. Mishra, J., et al. Identification of neutrophil gelatinase-associated lipocalin as a novel early urinary biomarker for ischemic renal injury. J Am Soc Nephrol 14, 2534-2543 (2003).
9. Barasch, J. & Mori, K. Cell biology: iron thievery. Nature 432, 811-813 (2004).
10. Flo, T. H., et al. Lipocalin 2 mediates an innate immune response to bacterial infection by sequestrating iron. Nature 432, 917-921 (2004).
11. Goetz, D. H., et al. The neutrophil lipocalin NGAL is a bacteriostatic agent that interferes with siderophore-mediated iron acquisition. Mol Cell 10, 1033-1043 (2002).
12. Lakso, M., et al. Efficient in vivo manipulation of mouse genomic sequences at the zygote stage. P Natl Acad Sci USA 93, 5860-5865 (1996).
13. Chassin, C., et al. Renal collecting duct epithelial cells react to pyelonephritis-associated Escherichia coli by activating distinct TLR4-dependent and -independent inflammatory pathways. Journal of immunology (Baltimore, Md.: 1950) 177, 4773-4784 (2006).
14. Yu, J., Carroll, T. J. & McMahon, A. P. Sonic hedgehog regulates proliferation and differentiation of mesenchymal cells in the mouse metanephric kidney. Development (Cambridge, England) 129, 5301-5312 (2002).
15. El-Achkar, T. M. & Dagher, P. C. Renal Toll-like receptors: recent advances and implications for disease. Nat Clin Pract Nephrol 2, 568-581 (2006).
16. Rodriguez, C. I., et al. High-efficiency deleter mice show that FLPe is an alternative to Cre-loxP. Nature Genetics 25, 139-140 (2000).
17. Dooley, T. P., Miranda, M., Jones, N. C. & DePamphilis, M. L. Transactivation of the adenovirus EIIa promoter in the absence of adenovirus E1A protein is restricted to mouse oocytes and preimplantation embryos. Development (Cambridge, England) 107, 945-956 (1989).
18. Clausen, B. E., Burkhardt, C., Reith, W., Renkawitz, R. & Forster, I. Conditional gene targeting in macrophages and granulocytes using LysMcre mice. Transgenic Res 8, 265-277 (1999).
19. Rice, B. W., Cable, M. D. & Nelson, M. B. In vivo imaging of light-emitting probes. J Biomed Opt 6, 432-440 (2001).
20. Li, J. Y., et al. Scara5 is a ferritin receptor mediating non-transferrin iron delivery. Developmental cell 16, 35-46 (2009).
21. Xie, Y., et al. Identification of N-(quinolin-8-yl)benzenesulfonamides as agents capable of down-regulating NFkappaB activity within two separate high-throughput screens of NFkappaB activation. Bioorg Med Chem Lett 18, 329-335 (2008).
22. Gong, G., et al. Discovery of novel small molecule cell type-specific enhancers of NF-kappaB nuclear translocation. Bioorg Med Chem Lett 19, 1191-1194 (2009).
23. Zhu, X. D. & Sadowski, P. D. Cleavage-dependent ligation by the FLP recombinase. Characterization of a mutant FLP protein with an alteration in a catalytic amino acid. The Journal of biological chemistry 270, 23044-23054 (1995).
24. Sauer, B. Functional expression of the cre-lox site-specific recombination system in the yeast Saccharomyces cerevisiae. Molecular and cellular biology 7, 2087-2096 (1987).

### Example 3

The kidney is the principal regulator of internal homeostasis, clearing metabolic products, excess salts and water and secreting erythropoietin and vitamin D2 into the blood. Here a novel function of the kidney tubule, the rapid excretion of large amounts of Neutrophil Gelatinase Associated Lipocalin (NGAL), also called Siderocalin (Scn), is described in response to urinary tract infections (UTI). NGAL-Scn has been shown to inhibit the growth of selected laboratory strains of bacteria by capturing specific types of catecholate-siderophores, reducing their access to iron. Nonetheless, the functional activity of urinary NGAL-Scn (uNGAL-Scn) has remained uncertain because it is activated by both infectious and non-infectious stimuli in different parts of the kidney. In addition, pathogenic bacteria express many different types of siderophores that are not recognized by NGAL-Scn.

To examine the function of NGAL-Scn, a UTI model with a pathogenic bacterium was utilized. For the first time an example of molecular cross-talk at the host-pathogen interface as a result of segmental expression of TLR4 was found. When the origin of the kidney NGAL-Scn was sought, it was found that NGAL-Scn predominantly originated from kidney epithelia, rather than from bladder, and within the kidney NGAL-Scn was synthesized by a specialized cell in the nephron, called the alpha-intercalated cell. In fact, GFP-expressing bacteria demonstrated direct binding to the apical domain of these cells. This cell is of great interest, because while there are multiple types of intercalated cells, the alpha cell acidifies the urine. Given that acidification also inhibits bacterial growth, a new paradigm emerges from this work, indicating that the alpha intercalated cell is not only a regulator of acid-base balance but additionally it is a sensor of uropathogenic bacteria and an immune effector which secretes H⁺ and NGAL-Scn.

To test this idea directly, the growth of uropathogenic bacteria was measured in vivo in NGAL-Scn^{-/-} and wild type mice and it was found that NGAL-Scn was a critical protein of bacteriostasis, despite the fact that uropathogenic CFT073 express many different siderophores. In fact, bacteria exposed to NGAL-Scn upregulated a variety of iron transport genes, implying that the bacteria were starved for iron. Further, when the minimal growth media was acidified to mimic urine pH, a stronger inhibitory effect of NGAL-Scn was found, implying that the two products of the alpha cell worked together to inhibit bacterial growth.

These data demonstrate that the kidney is an integral part of the response not only to pyelonephritis, but to cystitis as well, and that the expression of NGAL-Scn is critical for the response. Hence, NGAL-Scn differs from the better known urinary antimicrobial peptides and proteins by its rapid and intensive induction from specialized cells, and acts to inhibit a specific nutrient pathway. These data establish a rationale for abundant NGAL-Scn secretion from the kidney in both septic and aseptic states, demonstrating that the kidney defends the urinary system from pathogenic bacteria via the exocrine delivery of NGAL-Scn.

It was demonstrated that the kidney was the dominant source of the uNGAL-Scn after aseptic ischemic injury to the kidney. In Paragas et al, Nature Medicine 2011, it was claimed that to be a useful “biomarker” NGAL-Scn must meet a number of criteria: (1), the protein must originate from injured cells; (2), there should be a dose-dependent response to damage; (3), the expression of the biomarker should be rapid; (4), and reversible when the acute phase of injury has terminated; (5), the expression of the protein should be conserved across many patient populations and various animal models; (6), and importantly, the biomarker should be a critical component of organ pathophysiology. Here data from the multicenter human observational studies were included, showing that NGAL-Scn responds in a dose dependent manner to the UTI. Further by creating NGAL-Scn ko, NGAL-Luc2 reporter mice, cross-transplant techniques with TLR mutants, it is now shown that in a septic injury to the kidney, NGAL-Scn is a critical component of organ pathophysiology serving to significantly blunt the growth of uropathogenic bacteria at the acute phase of a urinary tract infection by novel mechanisms.

Without being bound by theory, these findings will be of great interest to biomedical scientists working in the field of acute kidney injury because the data explain its abundant expression by the kidney and they further add to the utility of NGAL as a biomarker. The data will also be of interest to scientists who discovered the antimicrobial activity of NGAL-Scn using lab strains rather than pathogenic bacteria. The data can lead to new methods for treating urinary tract infections by the delivery of excess NGAL-Scn into the urinary system.

### Example 4

The numbers between parentheses below refer to the corresponding numbered reference(s) at the end of this Example.

The Kidney Defends the Urinary System from Infection by Secreting NGAL-Scn

Here we describe a novel mechanism that defends the urinary system from infection. Neutrophil Gelatinase Associated Lipocalin (NGAL)-Siderocalin (Scn) is the well known biomarker of kidney stress resulting from ischemia, sepsis, or nephrotoxins (1), but its activity in the urinary system is unexplored. NGAL-Scn is known to inhibit bacterial growth by binding catecholate-siderophores (2, 3), but whether it has an antimicrobial activity in vivo against urinary pathogens which express several types of siderophores is unknown. Moreover, in kidney injury, NGAL-Scn derived from specialized α-intercalated cells (α-ICs) (1), which have an undocumented relationship to kidney defense. To examine the function of NGAL-Scn, we used uropathogenic E. coli (UPEC) in two murine models. In cystitis, there was rapid induction of kidney NGAL-Scn despite the apparent lack of invasion of the upper tracts. In pyelonephritis, bacteria entered the nephron and directly bound to α-ICs, which, in turn, synthesized NGAL-Scn by a TLR4-dependent mechanism. In vivo, NGAL-Scn was essential to rapidly clear infection, likely by starving bacteria of iron. These data provide a rationale for NGAL-Scn expression in kidney diseases and demonstrate that specialized kidney cells defend the lower urinary system from pathogenic bacteria by the exocrine delivery of NGAL-Scn.

Urinary tract infections (UTIs) are one of the most prevalent and resource-taxing diseases in the USA with 13.3% (12.8 million) of women and 2.3% (2 million) of men infected annually (4). In 2000, there were approximately 11 million diagnoses of UTI (4), with uropathogenic E. coli (UPEC) representing 70-95% of these cases (5).

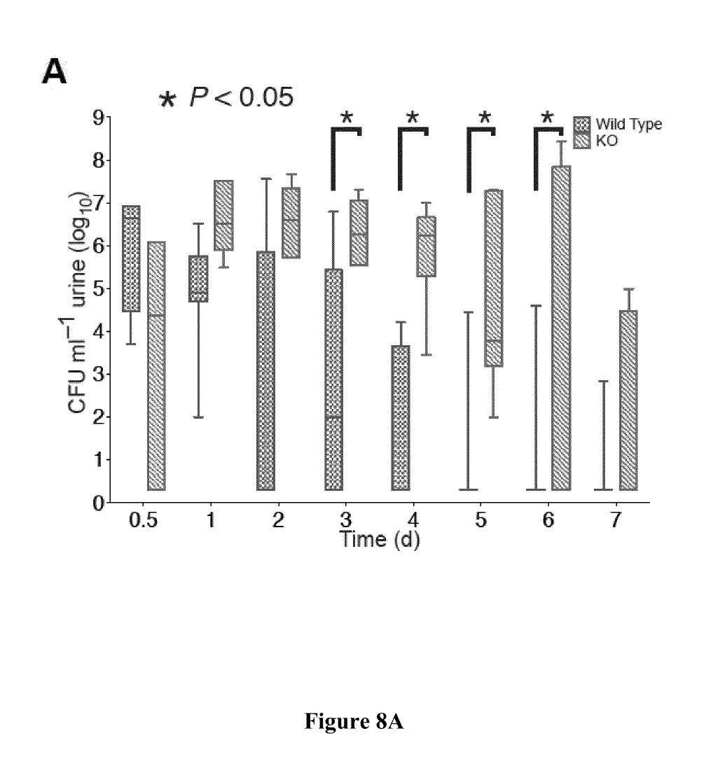

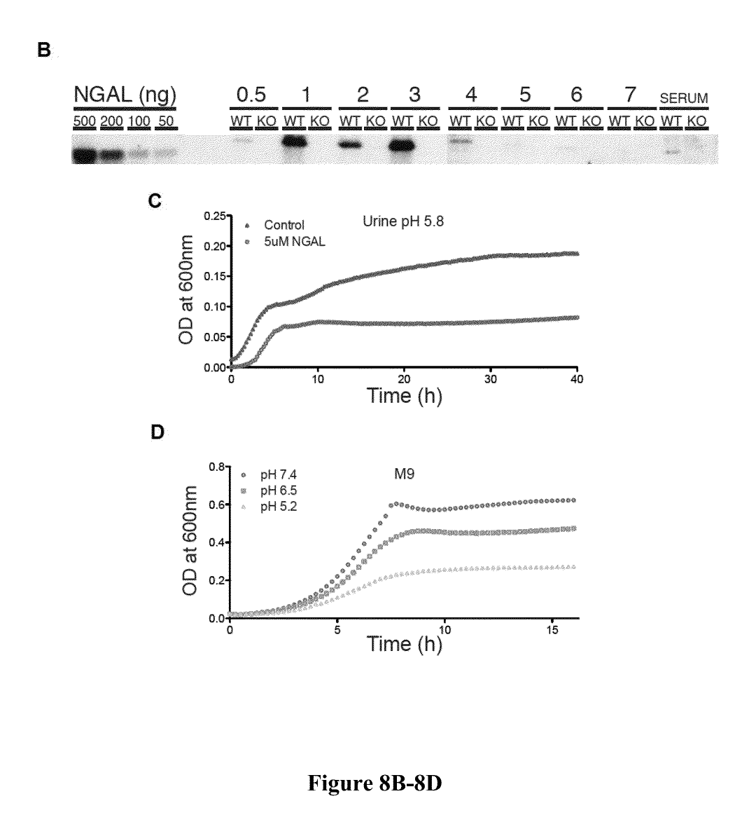

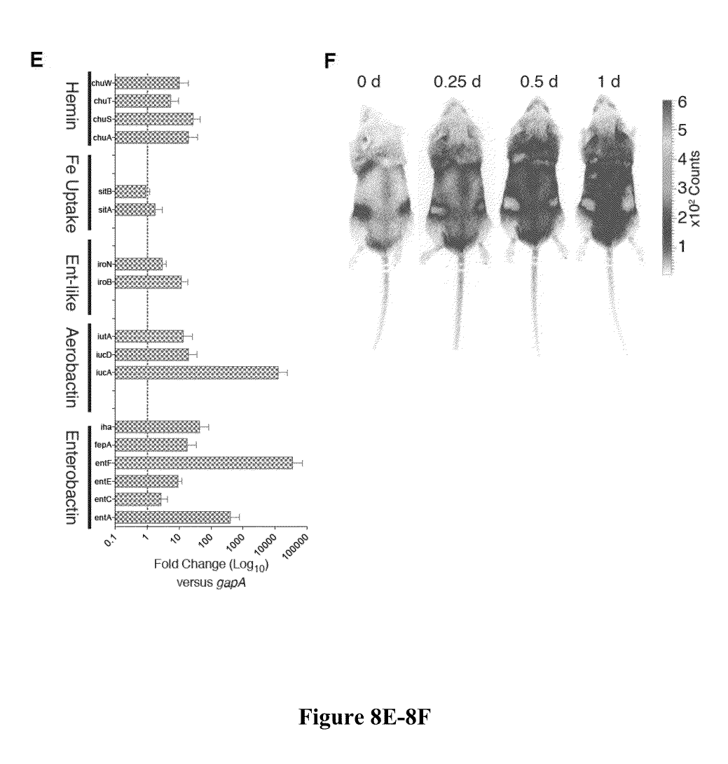

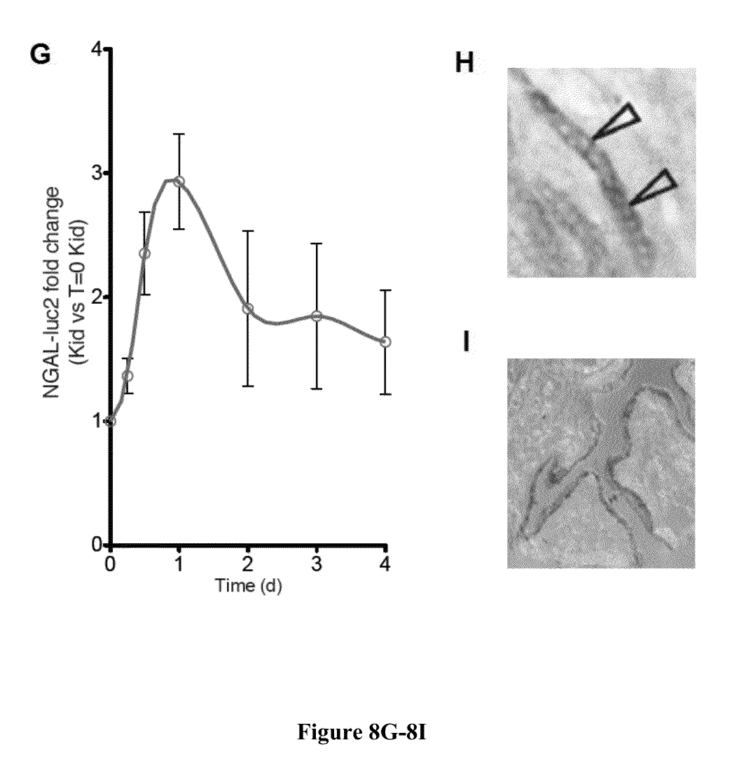

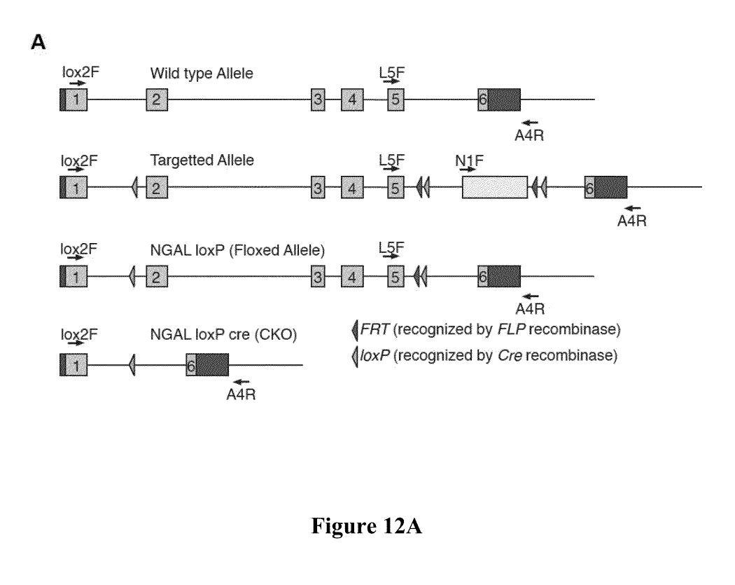

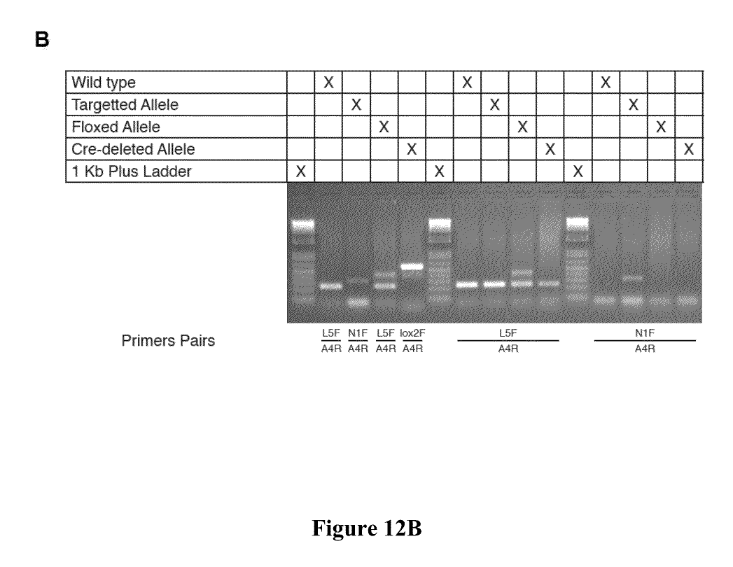

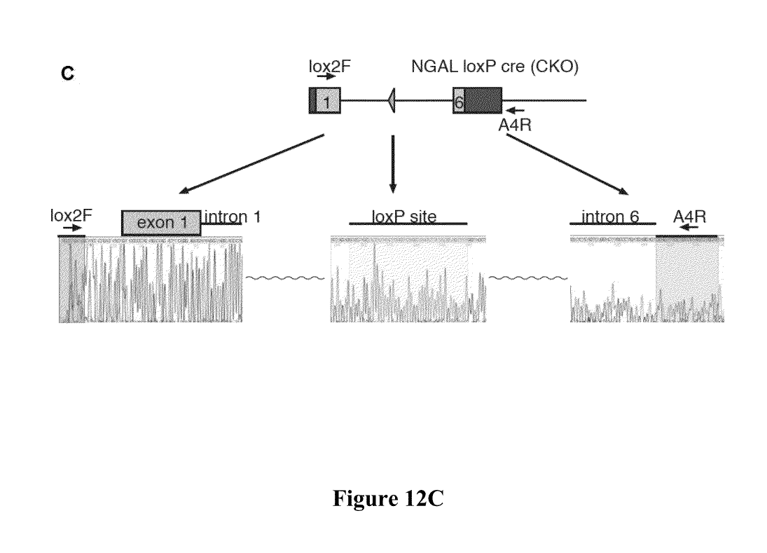

To determine the role of urinary (u) NGAL-Scn in UPEC induced acute cystitis, we created mice lacking NGAL-Scn. Ngal-Scn^{loxP/loxP} mice were generated and mated with EIIa-Cre mice (6) to generate a global knockout (Ngal-Scn^{-/-}) (Methods and FIG. 12). We challenged these mice with a small volume of a highly pyelonephritogenic UPECs (7) (CFT073 (8, 9), 20 μl of 5×10⁸ CFU ml⁻¹) by transurethral (TU) catheterization, and then longitudinally monitored uNGAL-Scn and urinary colony forming units (uCFU) for one week (FIG. 8A). In Ngal-Scn^{-/-} mice (blue checkered bar; n=7), uNGAL-Scn mirrored both peak uCFU levels (1-3 d) and the time to resolution of the acute infection (4-5 d, FIG. 8A, B). Ngal-Scn^{-/-} mice (red hashed bar; n=6) in contrast required a significantly longer time (>6 d) to resolve the infection; higher uCFU were identified in Ngal-Scn^{-/-} mice on the 3^{rd}-6^{th} day post-TU (P<0.05, Mann Whitney Test: FIG. 8A) implicating NGAL-Scn in the urogenital response to infection.

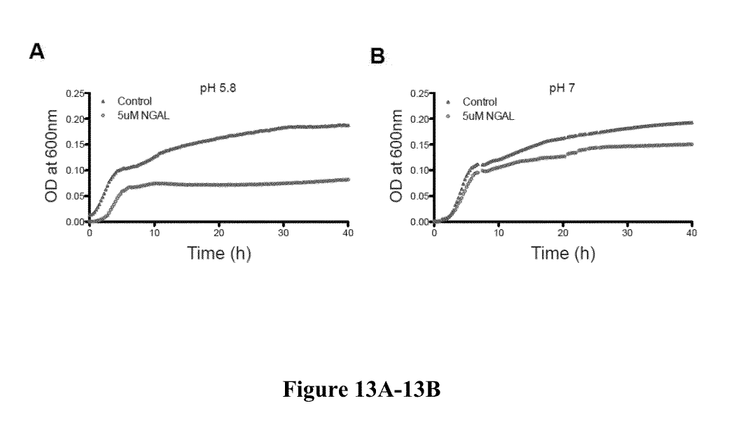

Next, we tested whether NGAL-Scn was sufficient to inhibit urinary bacterial growth. We used UPECs grown to log phase in an iron restricted minimal media (M9), and then subsequently transferred the bacteria into urine or M9. Growth in human urine (<pH 6.0) was similar to growth in acidified M9 (<pH 6.0; FIG. 8C, D). The addition of NGAL-Scn (5 μM, approximately a 5 fold molar excess compared to urine Fe) greatly inhibited bacterial growth in both urine (n=3 each; FIG. 8C red line) and M9 particularly in acidified M9 (FIG. 13 n=3 pH 5.2).

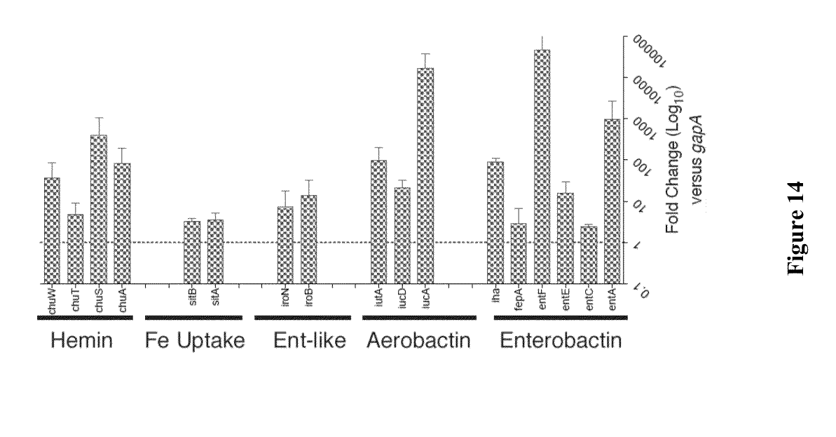

To determine whether NGAL-Scn induced bacterial iron starvation, we measured a series of iron acquisition systems (9, 10) that are regulated by iron load via fur, including catecholates enterochelin (ent genes) and salmochelin (iro genes), the hydroxamate aerobactin (iuc genes) (11, 12), their receptors, fepA, iroN (13-15), and iutA, respectively, and additionally the heme scavenging chu receptors (15). We found that the addition of NGAL-Scn (5 μM) to UPEC in M9 rapidly upregulated enterochelin regulon genes including synthetic enzymes (e.g. entA, and entF (16), 396.2 and 36294.5 fold) and receptors (e.g. fepA and iroN (17), 18.0 and 207 fold), aerobactin pathway genes including synthetic enzymes (e.g. iucA and D, 12568.5 and 19.0 fold) and receptors (e.g. iutA, 13.4 fold) and heme pathway genes (e.g. chuS, 26.9 fold), indicating that NGAL-Scn induced iron starvation and the widespread activation of compensatory pathways (n=3 FIG. 8E). In order to confirm the physiologic relevance of the growth conditions, we added small amounts of iron to M9 to match the urinary concentration (806 nM; Table 1 and Materials and Methods) and found that NGAL-Scn still activated enterochelin (e.g. EntF 2410 fold) and aerobactin genes (e.g. iucD and iutA, 16970 and 1.9 fold). Inhibition of iron uptake with DFO produced similar changes in gene expression (50 μM, FIG. 14), confirming the notion that NGAL-Scn induced iron-starvation. In sum, uNGAL-Scn is expressed within hours of the onset of infection in vivo, and it is required to limit bacterial growth during the early phases of acute cystitis, most likely by blocking iron acquisition.

TABLE 1
Iron Concentrations

| Bacterial Preparation of Mouse NGAL-Scn | | |
|---|---|---|
| NGAL-Scn concentration | 1.80 | mM |
| Fe concentration | 1.34 | μM |
| Fe in Media | | |
| M9 minimal media | 0.124 | μM |
| Human urine | 0.68 | μM |
| Mouse urine | 0.806 | μM |

| | |
|---|---|
| Molar Ratios of NGAL-Scn::Fe | |
| Bacterial Preparation of NGAL-Scn (5 μM) | 1343 |
| NGAL-Scn (5 μM) + M9 minimal media (undiluted) | 39.3 |
| NGAL-Scn (5 μM) + Human urine (undiluted) | 7.31 |
| NGAL-Scn (5 μM) + Mouse urine (undiluted) | 6.18 |

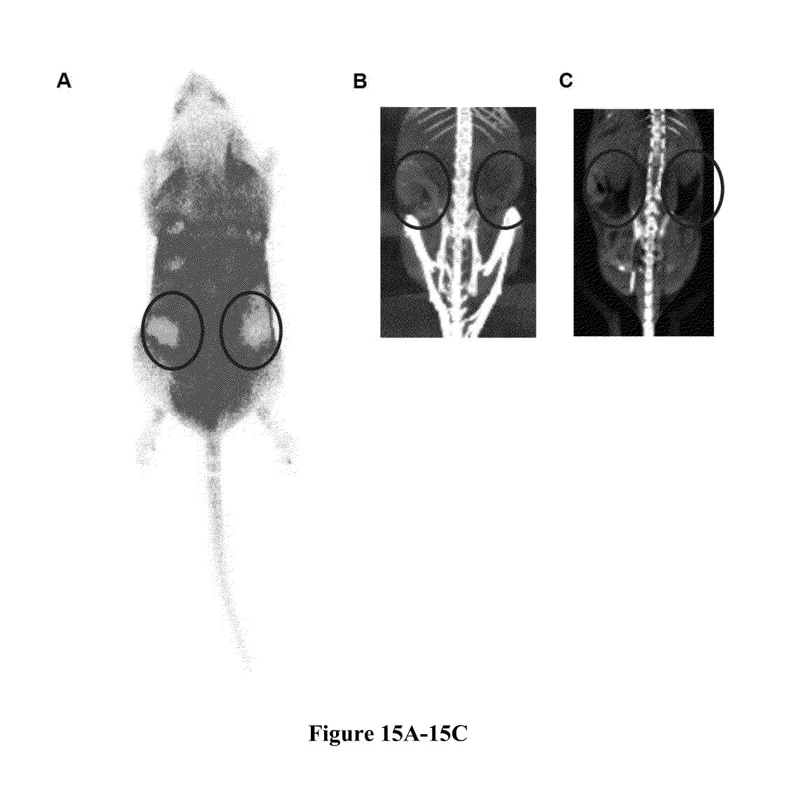

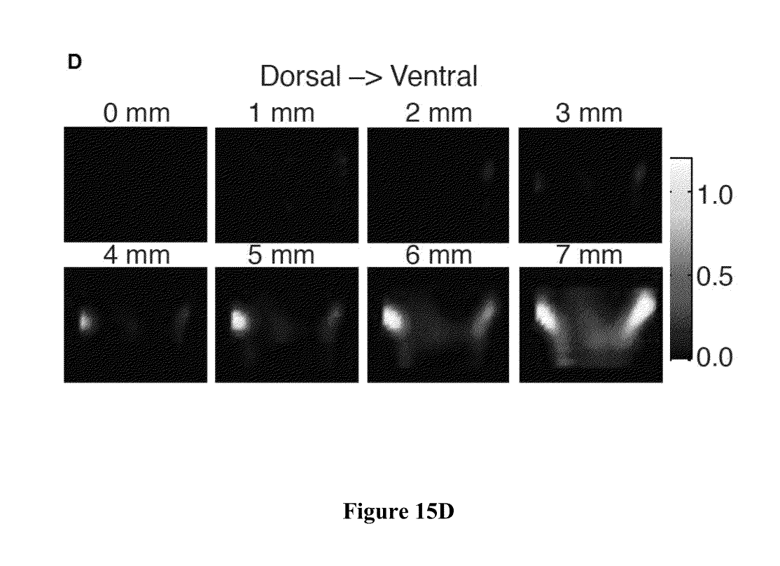

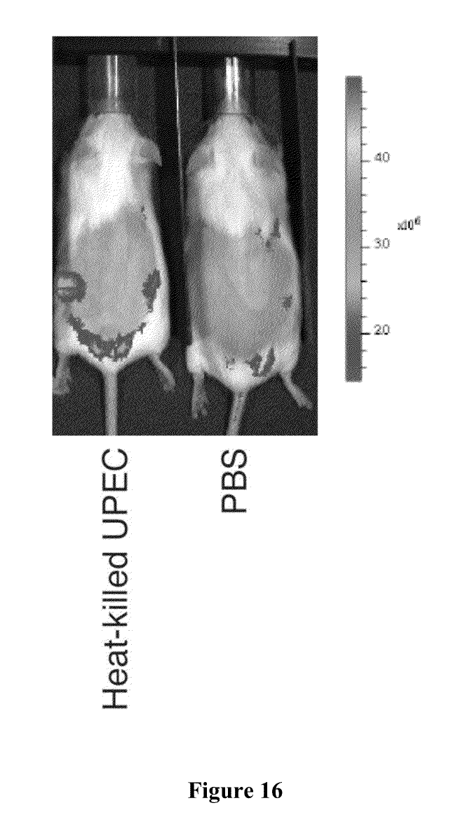

Cystitis is generally considered a disease of localized infection and inflammation (7), but since uNGAL-Scn has been reproducibly associated with kidney injury in humans (18-20) and in mice (1) as a result of both systemic septic (1, 18, 21-23) and aseptic injuries (1), we examined its anatomic source in mice with cystitis. UPECs were introduced into a bioluminescent reporter mouse NGAL-Luciferase2/mCherry (L2mC) (1) and images of NGAL-Scn expression were collected. A striking feature was the rapid response of the kidney to cystitis (within 0.25 d, FIG. 8F), followed by gradual resolution over 1-3 days. Kidney luminescence was 2.932±0.382 fold higher (n=10, P<0.0001) at 1 d compared to baseline (FIG. 8G). Three-dimensional analysis of bioluminescence coupled with nano-CT demonstrated that Ngal-Scn was expressed 4-7 mm deep from the dorsal surface within the kidney fossa (FIG. 15). These data were reproduced even when minute volumes of heat killed UPECs were introduced by TU into the mouse bladder (1×107 CFU; FIG. 16).

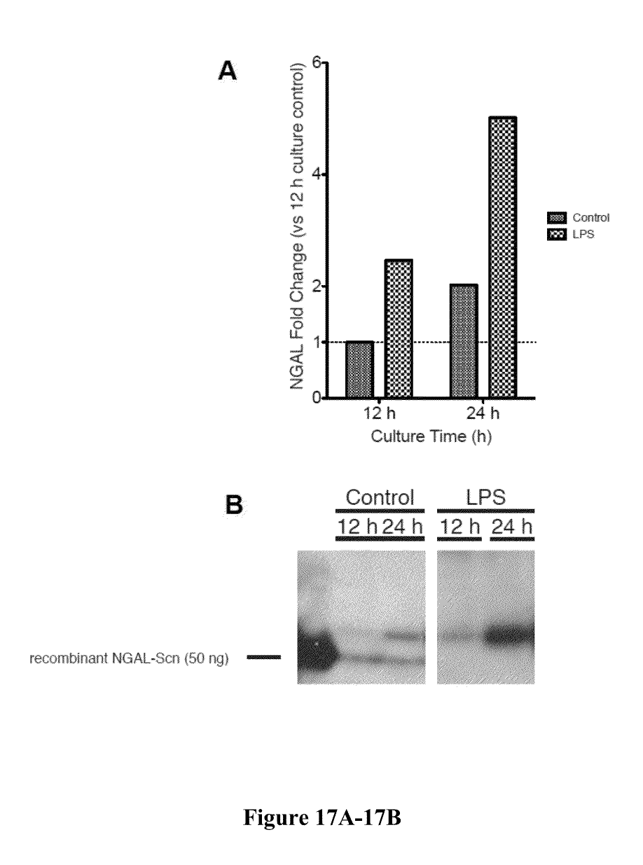

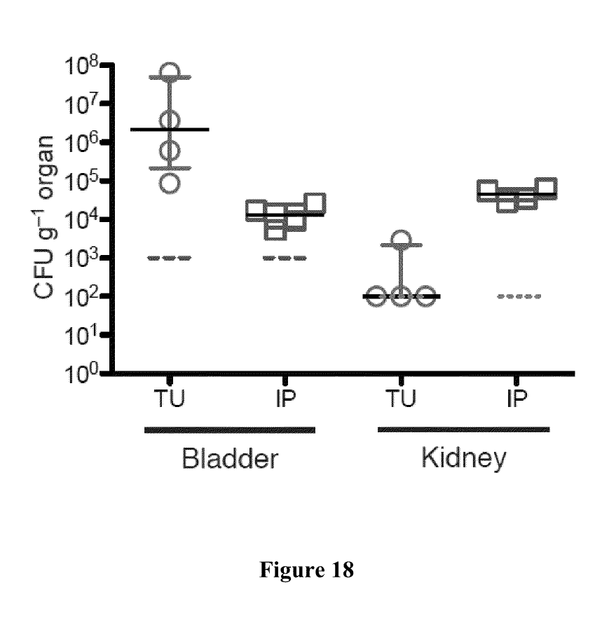

Because both the bladder and kidney might contribute to uNGAL-Scn expression, we performed in situ hybridization 1 d post-TU challenge with UPEC (20-30 μl of 5×10⁸ CFU ml⁻¹ i.e. ˜1×10⁷ total CFU). We found Ngal-Scn message in collecting duct epithelia (FIG. 8H) (1) and in a thin luminal layer of bladder epithelia (FIG. 8I), but the majority of Ngal-Scn transcripts were found in the kidney (FIG. 8J). Moreover, in a controlled experiment, bladders explanted from mice treated with LPS (1 mg kg⁻¹) secreted approximately 10 ng per bladder over 12 h (FIG. 17), a small percentage of even a single collection of urine (250 ng ul⁻¹) from the treated mouse. In sum, while many types of genitourinary epithelia can express uNGAL-Scn in response to cystitis, the kidney was a dominant source of NGAL-Scn, even though kidney bacteria fell at, or below, our limit of detection (10² CFU kidney⁻¹; FIG. 18), and an inoculum less than 50 μl is not likely to generate gross vesico-ureteral reflux (VUR) (24). Moreover, heat-killed bacteria, which are not predicted to ascend (24), were equally effective at inducing uNGAL-Scn in our cystitis model (FIG. 16). Hence, in acute cystitis it appears that bacterial components or a few ascending bacteria reach the kidney, interact with duct cells (24), and trigger NGAL-Scn expression by kidney epithelia.

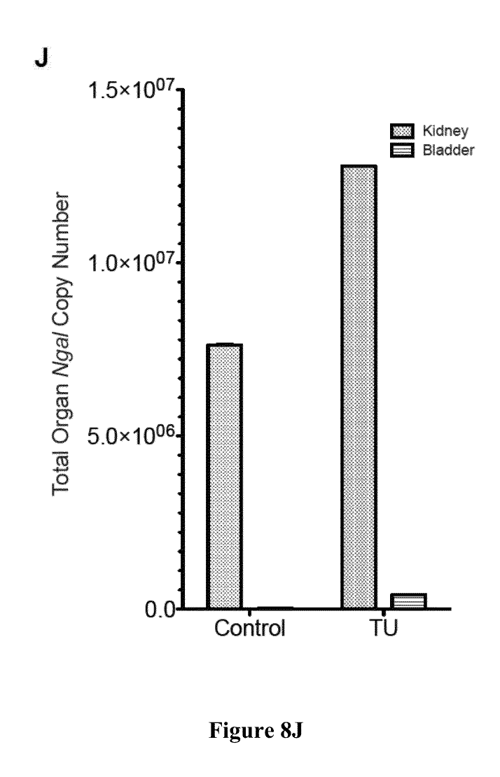

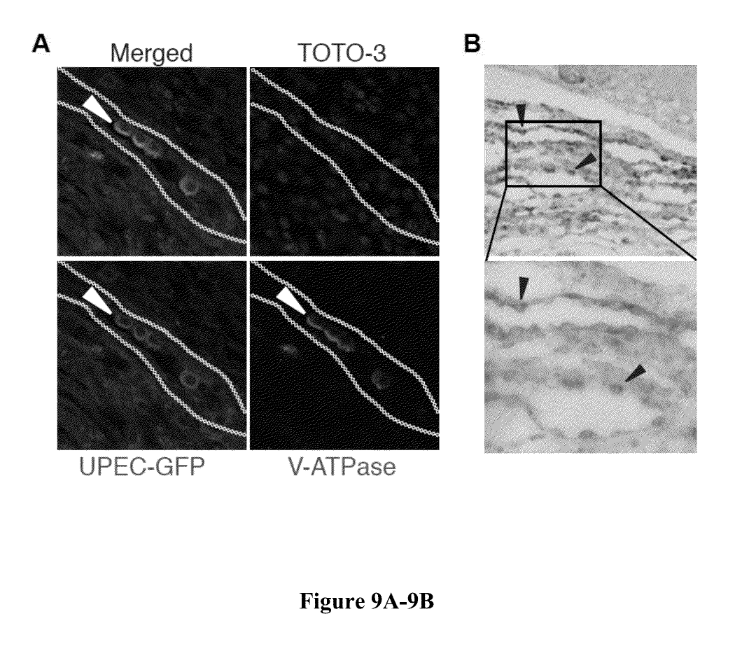

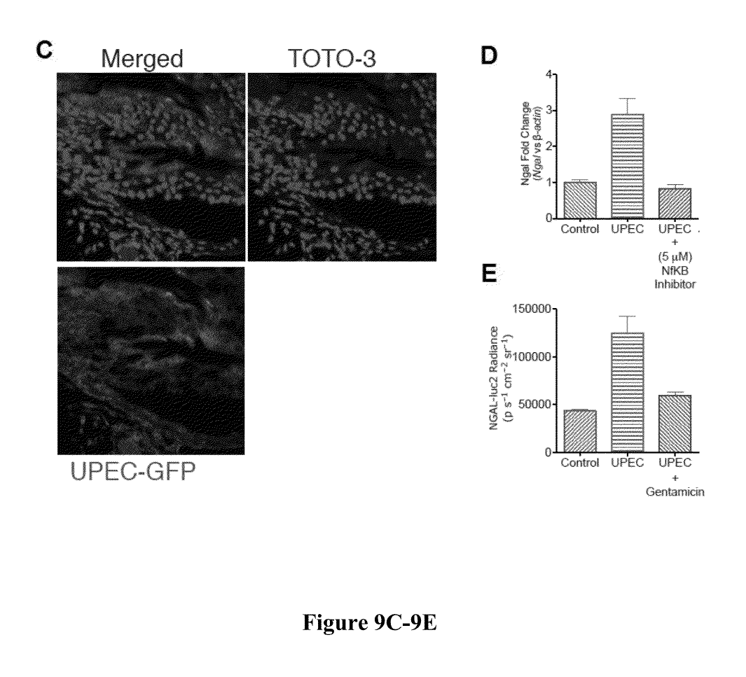

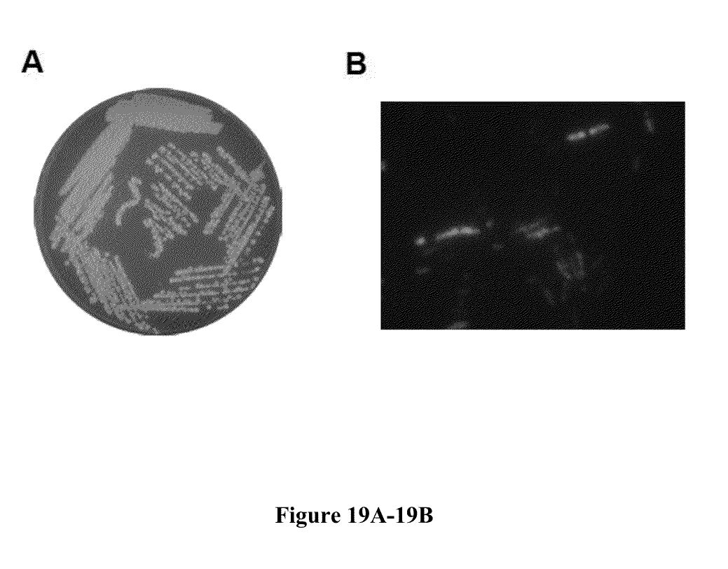

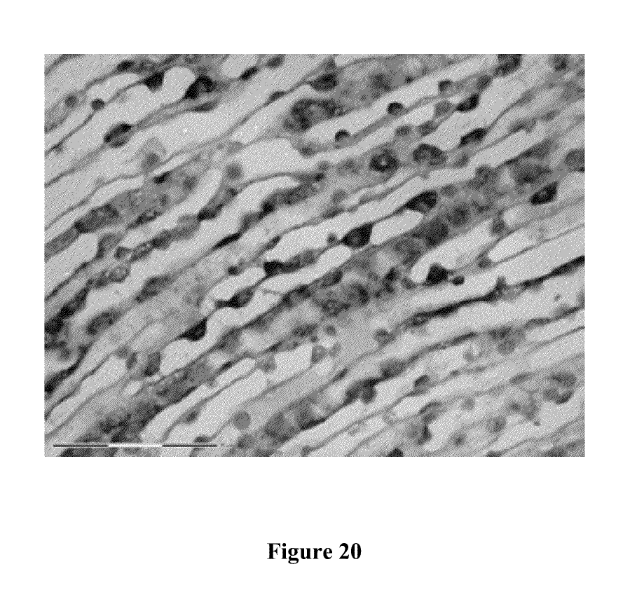

To test whether a direct interaction between bacteria and collecting duct cells was responsible for NGAL-Scn expression, we turned to C3H/HeN mice which are susceptible to UPEC mediated pyelonephritis (25). Using UPECs expressing GFP under control of the E. coli lac promoter (26), we found that UPEC-GFP bound to bladder epithelia, as well as entered the collecting ducts and specifically adhered to α-ICs with apically located V-ATPases (FIG. 9A, C and FIG. 19), resulting in the expression of Ngal-Scn (FIG. 9B). To examine the interaction of UPEC and α-IC cells further, we utilized an intercalated cell line which has characteristics of α-ICs (27). These cells responded to heat killed UPEC (approximately 5×10⁶ CFU) by expressing and secreting NGAL-Scn through an NF-κB sensitive pathway (inhibitor 5 μM) (28) (FIG. 9D). In addition, primary kidney cells isolated from Ngal-Luc2/mC reporter mice (1) also responded to UPECs by up regulating NGAL-Luc2 expression, while antibiotics reversed the induction (FIG. 9E) (1, 29, 30). Taken together, these data indicate that bacterial-epithelial interactions can drive IC expression of NGAL-Scn.

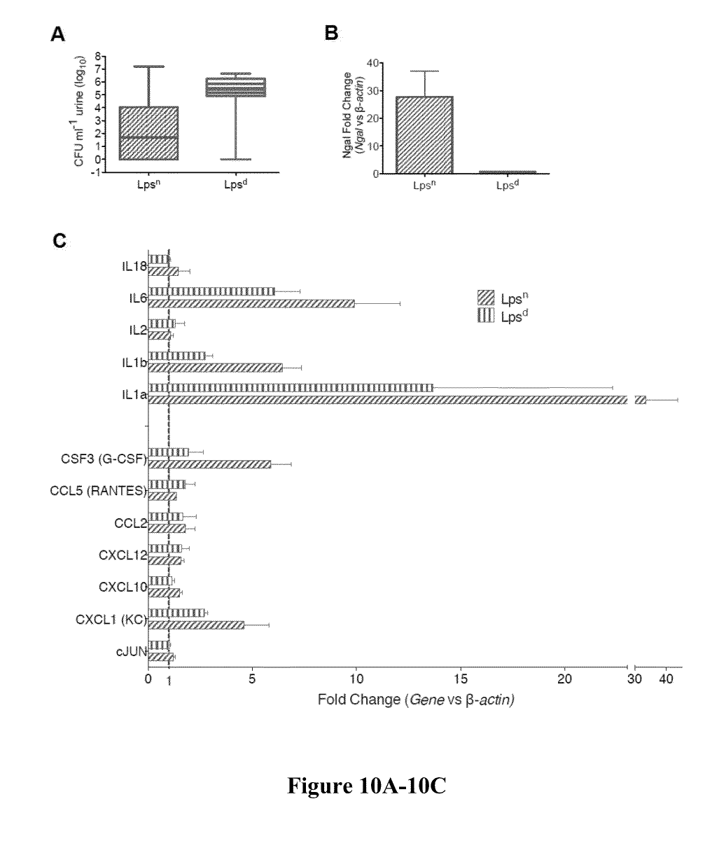

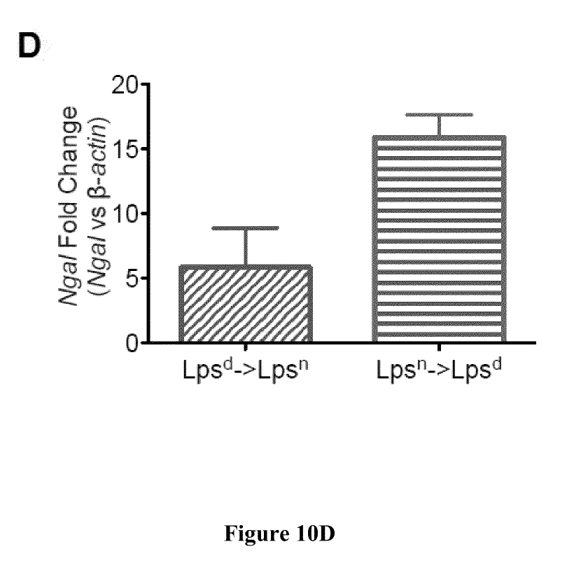

To identify elements of the pathway that detect UPEC, we evaluated whether IC could respond to a low dose of LPS (i.p. 1 mg kg⁻¹), a TLR4 agonist. LPS induced NGAL-Scn expression in IC in C3H/HeN mice (FIG. 19) in the same pattern as the pyelonephritis model (FIG. 9B). To assess the role of TLR4 further, we inoculated LPS-responsive C3H/HeN (Lps“) and C3H/HeOuJ (Lps”) mice and LPS-defective C3H/HeJ (Lps^{d}) mice by TU with 200 of 5×10⁸ CFU ml⁻¹ (n=15). We found that after 24 h, Lps^{d} (which have a mutation in the TLR4 receptor (31)) had higher urinary bacterial counts (FIG. 10A) and expressed much lower levels of kidney Ngal-Scn (FIG. 10B) and cytokines that amplify NGAL-Scn (e.g. IL-1β) (32) than paired Lpsⁿ (n=8) controls (FIG. 10C). To confirm that kidney expressed TLR4 was critical, we cross-transplanted Lps^{d} kidneys into nephrectomized Lpsⁿ controls and vice versa. Two weeks after graft maturation, when uNGAL-Scn and serum creatinine had normalized, we administered low dose LPS (1 mg kg⁻¹; FIG. 10D) and found that Lpsⁿ kidneys in Lps^{d} hosts had greater increases in Ngal-Scn (15.6±2.3 fold; n=3) than did Lps^{d} kidneys in Lpsⁿ hosts (4.5±2.6 fold; n=3) compared to untreated cross transplants. Limited induction in Lps^{d} kidneys was not surprising, because the mutation does not fully ablate TLR4 signaling, and wild type cells can invade the cross-transplanted kidney (1). Taken together, these results indicate that TLR4-dependent pathways are essential to regulate kidney NGAL-Scn probably by both direct NF-κB signaling (FIG. 9D) and indirectly as a result of cytokine signaling (FIG. 10C). Concurrently, TLR4 modulates the burden of urinary infection, by regulating the expression of NGAL-Scn in kidney epithelia.

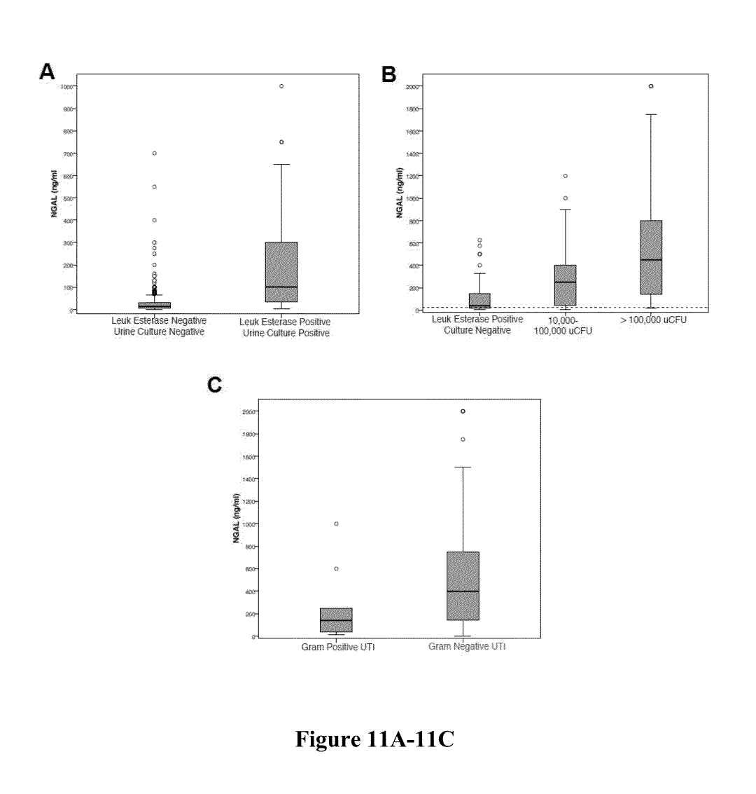

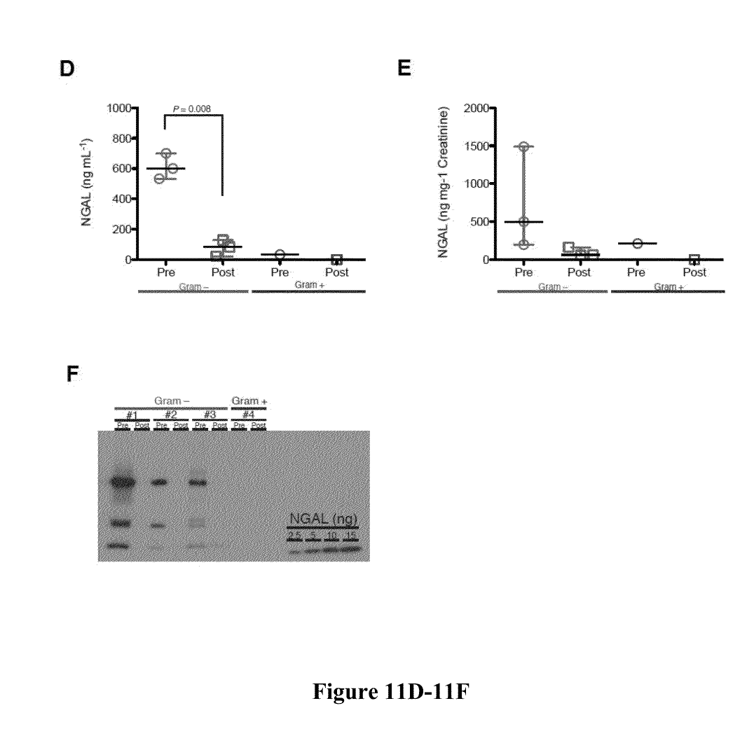

To determine the relevance of urinary NGAL-Scn to human infections, we analyzed a cohort of patients (n=1635) presenting to Emergency Departments in New York City and in Berlin (20). We identified a subset of patients without renal disease (n=651; see Materials and Methods), who were urine leukocyte esterase (LE⁺) and urine culture (Cx⁺) positive. These patients expressed significantly elevated uNGAL-Scn (P<0.0001) compared to LE⁻, Cx⁻ patients (237.4±289.53 ng ml⁻¹ n=43; vs 28.14±54.67 ng ml⁻¹, n=517, respectively; FIG. 11A). In fact, a dose-responsive relationship between uNGAL-Scn and increasing evidence of cystitis or pyelonephritis was found in an analysis of the entire cohort (LE⁺ and Cx⁻<LE⁺ with 10⁴-10⁵ CFUs<LE⁺ with >10⁵ CFU (P=0.013: LE⁺Cx⁻ vs LE⁺ with 10⁴-10⁵ CFU; P<0.001: LE⁺Cx⁻ vs LE⁺ with >10⁵CFU; P=0.022: 10⁴-10⁵ CFU vs>10⁵ CFU; FIG. 11B). Where data were available, we observed that uNGAL-Scn tended to be higher in patients presenting with Gram-negative (254.48±275.60 ng ml⁻¹ n=21) compared with Gram-positive infections (50.00±84.03 ng ml⁻¹, n=5, P=0.05, FIG. 11C). Moreover, in a limited separate study, when antibiotics were given to LE⁺ and Cx⁺ patients with dysuria, urgency, and hematuria, uNGAL-Scn levels fell back to normal by day 3 of therapy (n=3; P=0.008; FIGS. 11D, E and F). In contrast, a patient with a Gram-positive infection did not have elevated uNGAL-Scn levels (FIG. 11C, F).

In sum, both bladder and kidney epithelia responded to a small inoculum of bacteria as well as to overt upper tract infection. In fact, given that TLR4 mutants neither reduced their bacterial burden, nor expressed epithelial NGAL-Scn, it appears that ligands of TLR4 must reach the kidney to active this immune defense. We suspect that the process of reflux is variable in cystitis as a function of background (C57BL6 vs C3H) or bacterial burden, perhaps accounting for variable NGAL-Scn levels found in humans with urinary infections. Consequently, we suggest that cystitis and the first phase of pyelonephritis are distinguished only by the size of the kidney inoculum and the degree of NGAL-Scn induction (e.g. 10-fold higher in pyelonephritis).

Having ascended to the kidney, bacteria or their ligands directly adhered to IC (26). While there are multiple types of intercalated cells, we identified these bacterial sensors as α-ICs which secrete NGAL-Scn and H⁺ by apical ATPases (1). Indeed, bacterial growth was limited by both acidification (pH 4.5-6.0; FIG. 8C, D) (33-36) and by NGAL-Scn, which binds enterochelin even at pH 4.0 (2, 3). In this light, our data identify an unexpected innate immune response: α-IC cells not only regulate acid-base homeostasis (37) but additionally serve as antimicrobial effectors, secreting two factors (H⁺ and NGAL-Scn) which are likely to play synergistic roles in suppressing cystitis and pyelonephritis. Indeed α-IC also tonically secrete RNase7, a non-selective antimicrobial peptide (38).

While NGAL-Scn is best characterized with E. coli sHB101 and H9049, which depend solely on Ent (29), UPEC CFT073 expresses multiple mechanisms of iron capture (39, 40). Yet the surprising inhibitory activity of NGAL-Scn in vivo and in vitro implied a dominant role for Ent in the growth of UPEC. Hence, unlike the better known antimicrobial peptides (41) (i) NGAL-Scn is intensely upregulated in both septic and aseptic injuries of the kidney, providing a general “biomarker” (1, 20) of kidney injury that (ii) targets a specific pathway of iron acquisition rather than broad antimicrobial activities, yet (iii) is critical in defense against complex urinary pathogens.

We conclude that the kidney acts as an “exocrine organ” that senses the presence of damage—UPECs via TLR receptors—whereupon it secretes uNGAL-Scn from specialized cells to defend the urogenital tract from both pyelonephritis and cystitis.

### Methods

Mouse Husbandry.

Ngal-Scn^{loxP/loxP}, Ngal-Scn^{EII-Cre} C57BL/6, C3H/HeJ, C3H/HeOuJ, C3H/HeN, and Ngal-Luc2/mC mice were generated and analyzed by approved protocols.

Generation of Ngal-Sce^{loxP/loxP}

We created a targeting vector to delete exons 2-5 (a span of 2.1 Kb) because this region contains important caliceal amino acids 158,159,160,161. Using a C57BL/6J library (RPC)-23; CHOR1) and bacterial recombineering, a single LoxP was inserted into intron 1 and FRT-loxP-neo-FRT-loxP was inserted into intron 5. The targeting construct was 14.2 kb consisting of a (5′) 7.8 kb long homology arm, a loxP in intron 1, exons 2-5, a 2 kb pGK-neo cassette flanked by FRT-loxP-neo-FRT-loxP and finally a (3′) 2.3 kb short homology arm. A third loxP site provided a backup in case FLP was inefficient. The targeting vector was electroporated and ES clones were selected with neomycin and validated by PCR. 13 heterozygous F1 pups carrying targeted alleles, Ngal-Scn^{+/loxP-flp} were generated from F0 mice, and crossed with the FLP deleter (βactin promoter-FLP B6; SJLTg(ACTFLPe)9205Dym/J; JAX Mice Stock#003800) which had been backcrossed to C57BL/6 for 5 generations to reduce genetic heterogeneity. The offspring βactin-flp; Ngal-Sce^{loxP/+} mice were mated with C57BL/6 to eliminate βactin-flp and then brother-sister mating followed to produce Ngal-Scn^{loxP/loxP} mice. The Ngal-Scn allele was deleted by breeding the Ngal-Scn^{loxP/loxP} to EIIa-Cre mouse (B6.FVB-Tg(EIIa-cre) C5379Lmgd/J, JAX Mice Stock #003724) (6, 42).

Imaging of Living Ngal-Scn-Luc2/mC Reporter Mice.

Ngal-Luc2/mC reporter mice (1) were injected i.p. with 150 mg/kg of D-luciferin (Caliper Life Sciences) in PBS (pH 7.0) anesthesized (2.5% isofluorane) and imaged for 30 s using the PhotonIMAGER optical imaging system (Biospace Labs) with open excitation and emission filters for luminescence and fluorescence, respectively. Regions of interest (ROIs) were quantified using bundled photoacquisition software (BioSpace Labs). A CCD camera digitizer measured the ROIs and counts were converted to physical units of radiance in photons/s/cm2/steradian.

3D Image Analysis and CT imaging of Ngal-Scn-Luc2/mC Reporter Mice.

Ngal-Luc2/mC reporter mice¹ were immobilized on an optical imaging bed and placed into a 4-view module to capture multi-angle images of the optical signal (dorsal, ventral and both lateral views of an entire animal) at five wavelength bands of 50 nm width between 550 nm and 720 nm. The image acquisition time was 120 seconds for each wavelength band. 3D image reconstruction utilized an expectation-maximization (EM) method for the 3D image reconstruction (42). This algorithm utilizes a light propagation model based on simplified spherical harmonics (SP3) equations of third-order (43). After optical imaging, the immobilized animal was transferred to a NanoSPECT/CT camera (Bioscan, Washington, D.C.). CT scans were performed at standard frame resolution using a tube voltage of 45 kVp, 1000 ms/projections, 240 projections/rotation. Each acquisition was approximately 4 min. The CT data was reconstructed using InVivoScope post-processing software (Bioscan).

Kidney Ischemia and Cross Transplantation

Surgical cross-transplants (1, 44) were monitored for two weeks until serum creatinine stabilized to 0.2 mg dL⁻¹ and uNGAL-Scn was undetectable prior to ip challenge with LPS (1 mg kg⁻¹).

Urinary Tract Infections.

Female C57BL/6, Ngal-Scn^{-/-}, C3H/HeJ, C3H/HeN and C3H/HeOuJ mice at an age of 8-16 weeks were used. We placed 20 μl of a bacterial suspension or heat killed bacteria (1×10⁷ CFU) into the bladder of anesthesized mice through a soft polyethylene catheter (Intramedic, 0.61 mm outer diameter). CFUs in kidney homogenates or in urine (collected directly from mice) were quantified by serial dilution on LB agar plates. Datasets and samples were also obtained according to IRB protocols with informed consent (20) from the Experimental and Clinical Research Center, Charité-Universitätsmedizin, Max Delbruck Center for Molecular Medicine and Helios Clinic, Berlin, Germany (20) and Columbia University Medical Center. Analyses utilized SPSS version 16.0. Continuous data were log-transformed prior to analysis but presented as non-log-transformed values. T-test for unequal variances was used for comparisons (Welsh's T-test). Prism 5 was used for all other data analysis (GraphPad Software).

Inhibition of Bacterial Growth In Vitro

A single colony of CFT073 was selected from a plate and grown in M9 to log phase. Bacteria were pelleted and resuspended in either M9 or urine and monitored in a 96 well plate on a Tecan 200 Promicroplate reader for up to 72 h. Notably when CFT073 were grown to log phase in LB and then transferred to M9 or urine there was more variability in our results probably from iron carry-over (38).

NGAL-Scn Protein Production

Recombinant protein was produced in BL21 E. coli transformed with Ngal-Scn cDNA lacking 29aa signal sequence (pGEX-4T-3-vector) and grown for 16 h at 37° C. in TB supplemented with 150 μM iron to inhibit endogenous production of enterochelin. IPTG (0.2 mM final concentration) was added for 5 h. Bacterial pellets were lysed by sonication in lysis buffer, followed by Triton-X 100 (0.5%) treatment for 30 min on ice. Supernatant was collected after high speed centrifugation and filter sterilized (0.45 μm). NGAL-Scn-GST was purified by binding to Glutathione Sepharose beads followed by cleavage of the GST tag with thrombin. Released protein was fractionated by a Sephacryl S100HR column. Enterochelin:Fe capture by NGAL-Scn was tested (Emc Microcollections gmbh) at a 3:1 Enterochelin:Fe::Ngal-Scn ratio. The complex was washed 5 times on a 10 k centriprep centrifugal filter and binding detected by its coloration, which was lacking in the absence of additional Enterochelin or NGAL-Scn protein.

Isolation and Culture of Cells

Luc2/mC di-fusion reporter mice (8-12 weeks of age) were perfused with PBS and kidney cells were isolated with collagenase (2 mg ml⁻¹; Sigma), and cultured (1×10⁵ well⁻¹ in 24-well plates; Falcon) in DMEM/F12 medium supplemented with 10% FBS, 1% penicillin-streptomycin, and 46 mg/l L-Valine for 24 h. The cells were treated for 24 h either with 5 μl of 10⁹ CFU ml⁻¹, i.e. approximately 5×10⁶ CFU E. coli CFT073 heat killed by boiling (30 min) or with Lipid A (“LPS” 4 μg/ml) and NF-κB inhibitor, Analogue 31 (5 μM) (28). Luciferase substrate (Dual-Glo Luciferase Assay System; Promega) was added and luminescence from Luc2 and fluorescence from mC (excitation 500-550 nm and emission 575-650 nm) were imaged in a Xenogen IVIS optical imaging system.

Rabbit intercalated cells Clone C were obtained from S. Vijayakumar (University of Rochester), maintained at 32° C., and then seeded on Corning Transwell #3412 at a density of 5×10⁵ cells/cm² (high density) in DMEM/F12 50:50 (Mediatech Cellgro, MT10090CV) with 10% heat inactivated FBS (Invitrogen), 1% Penicillin-Streptomycin, 20 mg/L Hydrocortisone, and an Insulin, Transferrin, and Selenium supplement (Lonza) at 40° C. (to inactivate the T-antigen). Cells were serum starved prior to treatments and RNA was extracted using an Ambion kit (AM1560) with DNAse digestion.

Iron Levels

Pooled C3H/HeN urine (n=15) was collected by clean catch and centrifuged for 10 min at 12,000 rpm. Urine, protein, and media Fe concentration were measured by a Graphite Furnace Atomic Absorption Spectrophotometer (GFAAS), model Analyst 800 (Perkin Elmer) by the Trace Metals Core Facility at the Columbia University Mailman School of Public Health.

Western Blot

Urines were analyzed using non-reducing 4-15% tris-HCL gels (Bio-Rad, Laboratories, Inc. Hercules, Calif.) and monoclonal human (1:1000; Enzo Lifesciences, BPD-HYB-211-01-02) or mouse antibodies (1:1000, R&D Systems, AF1857). NGAL-Scn was reproducibly detected to 0.4 ng/lane. NGAL-Scn protein was semi-quantified by comparison with mouse or human NGAL-Scn protein standards (0.2-10 ng) using Image-J software (NIH).

In Situ Hybridization and Immunohistochemistry.

Ngal-Scn RNA was detected using digoxigenin-labeled antisense riboprobes (Roche Applied Biosystems) from cDNAs encoding Ngal-Scn (exon 1-7, 566 bp) by linearization with XhoI followed by T7 RNA polymerase as previously described45. Frozen and paraffin-embedded sections were used for immunohistochemical analyses. Anti-vATPase B1/2 (Santa Cruz Biotechnology 1:50) and nuclear stains DAPI and TOTO3 (1:1000) were used on frozen sections.

Real-Time PCR Analysis.

Total RNA was isolated with the mirVANA (for eukaryotic cells) or ribopure (for bacteria) RNA extraction kits (Ambion). First strand cDNA was synthesized with Superscript III (Invitrogen). Real-time PCR was performed in a 7500 Fast (Applied Biosystems) with a SYBR green supermix reagent (Fisher) and primers (Table 2) using β-actin (eukaryotic cells) and gapA (for bacteria) as internal controls. Fold amplification of transcripts was measured by the AACT method.

TABLE 2
List of PCR and qPCR primers

| Primer | Ngal-Scn flox/flox |
|---|---|
| Name | genotyping primers |
| lox2F | 5′- AAGACTCAACTCAGAACTTGATCC -3′ |
| A4R | 5′- GAGGAAGCTTGGACAGGAATCTGG -3′ |
| L5F | 5′- ACGACAACATCATCTTCTCTGTCC -3′ |
| N1F | 5′- TGCGAGGCCAGAGGCCACTTGTGTAGC -3′ |
| Ngal-ScnR | 5′- GTCCTTCTCACTTTGACAGAAGTCAGG -3′ |
| Ngal-ScnF | 5′- CACATCTCATGCTGCTCAGATAGCCAC -3′ |
| Gene | |
| Symbol | Q-PCR primers |
| Ngal-Scn | |
| Forward | 5′- CTCAGAACTTGATCCCTGCC -3′ |
| Reverse | 5′- TCCTTGAGGCCCAGAGACTT -3′ |
| bact | |
| Forward | 5′- CTAAGGCCAACCGTGAAAAG -3′ |
| Reverse | 5′- TCTCAGCTGTGGTGGTGAAG -3′ |
| gapA | |
| Forward | 5′- AAGTTGGTGTTGACGTTGTCGC -3′ |
| Reverse | 5′ - AGCGCCTTTAACGAACATCG -3′ |
| chuA | |
| Forward | 5′- AGCGTGTTGAGATTGTTCGC -3′ |
| Reverse | 5′- AAACCACTGCTTTGTCCTTCCTGC- 3′ |
| chuS | |
| Forward | 5′- CTGTTTCTCAATCAATGGGCCAGTG -3′ |
| Reverse | 5′- TATGCCACCGACAATACCGATATGG -3′ |
| chuT | |
| Forward | 5′- AGCTGGACTTTTAGCGTAACGGCTG -3′ |
| Reverse | 5′- CGACATCTTATCCAGGAGAAACCGC -3′ |
| chuW | |
| Forward | 5′- GCCACTGGCCCCTGATTGTGAA -3′ |
| Reverse | 5′- AATCCGCAAGAAAATGGCCCG -3′ |
| entA | |
| Forward | 5′- TAGCTGAAACGGAGCGACTGGACG -3′ |
| Reverse | 5′- ACGTCGGCGGTGCGTTTAACCT -3′ |
| entC | |
| Forward | 5′- CCAAAGCGCAGGGCATCAAA -3′ |
| Reverse | 5′- AAAACAAGCTTCCGCACGCCG -3′ |
| entE | |
| Forward | 5′- AGCGGGGATGATTTCCTCAACAC -3′ |
| Reverse | 5′- GCTGAGGATTTTACTGCCACGCC -3′ |
| entF | |
| Forward | 5′- CGCTGTTCGGTCCGGTAGTCAA -3′ |
| Reverse | 5′- TGAACTGGCCCTGTTCCCGGAT -3′ |
| fhuA | |
| Forward | 5′- ACGGCCAAAGCCAGAATAAC -3′ |
| Reverse | 5′- TTACCGTAAAGCACGGAAACCG -3′ |
| iroB | |
| Forward | 5′- CCGGTCTGGATTCCGAAGCTGGTTA -3′ |
| Reverse | AGACCATCTGGTGGAGTTTGCCG -3′ |
| iroN | |
| Forward | 5′- ATTACCAAACGTCCCACCAACG -3′ |
| Reverse | 5′- AAACGCGTGGTAAGAGCATCAC -3′ |
| iucA | |
| Forward | 5′- CCCATACGCAACAGGCAATTGATG -3′ |
| Reverse | 5′- CACCCTGCGCCTAAGTCTCATGA -3′ |
| iucD | |
| Forward | 5′- CTTCAGCGAAGAAATGGCAGACCA -3′ |
| Reverse | 5′- TGATATACCGGTCGTGATGCAAACC -3′ |
| sitB | |
| Forward | 5′- GGCGTTACAGAAAAACCTGGTTGCC -3′ |
| Reverse | 5′- ATGGGTATGTTGCGTATCGCCAAA -3′ |
| sitA | |
| Forward | 5′- GCACTCACCTGCTCGATCGCAT -3′ |
| Reverse | 5′- TCTCATCCATAACCAAGCCTGGTGC -3′ |

Taqman Genes

| Gene | |
|---|---|
| Symbol | Assay ID |
| Aco1 | Mm00801417_m1 |
| Actb | Mm01205647_g1 |
| Arnt | Mm00507836_m1 |
| Ccl2 | Mm00441242_m1 |
| Ccl5 | Mm01302427_m1 |
| Cd14 | Mm00438094_g1 |
| Cebpa | Mm00514283_s1 |
| Cebpb | Mm00843434_s1 |
| Clec4e | Mm00490873_m1 |
| Csf3 | Mm00438334_m1 |
| Cxcl1 | Mm01354329_g1 |
| Cxcl10 | Mm00445235_m1 |
| Cxcl12 | Mm00445552_m1 |
| Epas1 | Mm00438717_m1 |
| Fth1 | Mm00850707_g1 |
| Ftl1 | Mm03030144_g1 |
| Hamp | Mm00519025_m1 |
| Havcr1 | Mm00506686_m1 |
| Heph | Mm00515970_m1 |
| Hif1a | Mm00468869_m1 |
| Hsf2 | Mm00434027_m1 |
| Hspa1a | Mm01159846_s1 |
| Icam1 | Mm00516023_m1 |
| Ikbkb | Mm01222247_m1 |
| Il18 | Mm00434225_m1 |
| Il1a | Mm00439621_m1 |
| Il1b | Mm00434228_m1 |
| Il1r1 | Mm00434237_m1 |
| Il2 | Mm00434256_m1 |
| Il6 | Mm00446191_m1 |
| Jun | Mm00495062_s1 |
| Keap1 | Mm00497268_m1 |
| Lcn2 | Mm01324470_m1 |
| Nfe2l2 | Mm00477784_m1 |
| Nfkbia | Mm00477798_m1 |
| Nfkbib | Mm00455849_m1 |
| Nfkbil1 | Mm00447990_m1 |
| Nfrkb | Mm00555264_m1 |
| Pglyrp1 | Mm00437150_m1 |
| Ptgs2 | Mm00478377_g1 |
| Rel | Mm01239661_m1 |
| Rela | Mm00501346_m1 |
| Scara5 | Mm00512272_m1 |
| Slc11a2 | Mm00435363_m1 |
| Slc40a1 | Mm00489837_m1 |
| Spn | Mm00493383_s1 |
| Tfrc | Mm00441941_m1 |
| Ticam2 | Mm01260003_m1 |
| Tlr11 | Mm01101924_s1 |
| Tlr2 | Mm00442346_m1 |
| Tlr4 | Mm00445273_m1 |
| Tlr5 | Mm00546288_s1 |
| Tlr9 | Mm00446193_m1 |
| Tnf | Mm00443258_m1 |
| Tnfrsf1a | Mm00441883_g1 |
| Trfr2 | Mm00443703_m1 |

### REFERENCES

Paragas, N., et al. The Ngal reporter mouse detects the response of the kidney to injury in real time. Nature medicine 17, 216-222 (2011).
2. Goetz, D. H., et al. The neutrophil lipocalin NGAL is a bacteriostatic agent that interferes with siderophore-mediated iron acquisition. Mol Cell 10, 1033-1043 (2002).
3. Bao, G., Clifton, M., Hoette, T., Mori, K., Deng, S., Qiu, A., Viltard, M., Williams, D, Paragas, N, Leete, T, Kulkarni, R., L1, X., Lee, B., Kalandadze, A., Ratner, A., Pizarro, J., Schmidt-Ott, K., Landry, D., Raymond, K., Strong, R. and Barasch, J. Iron traffics in circulation bound to a siderocalin (Ngal)-catechol complex. Nat Chem Biol 6, 7 (2010).
4. Litwin, M. S., et al. Urologic diseases in America Project: analytical methods and principal findings. J Urol 173, 933-937 (2005).
5. Brzuszkiewicz, E., et al. How to become a uropathogen: comparative genomic analysis of extraintestinal pathogenic Escherichia coli strains. P Natl Acad Sci USA 103, 12879-12884 (2006).
6. Lakso, M., et al. Efficient in vivo manipulation of mouse genomic sequences at the zygote stage. P Natl Acad Sci USA 93, 5860-5865 (1996).
7. Gupta, K., et al. International clinical practice guidelines for the treatment of acute uncomplicated cystitis and pyelonephritis in women: a 2010 update by the infectious diseases society of america and the European society for microbiology and infectious diseases. Clin Infect Dis 52, e103-120 (2011).
8. Mobley, H. L., et al. Pyelonephritogenic Escherichia coli and killing of cultured human renal proximal tubular epithelial cells: role of hemolysin in some strains. Infect Immun 58, 1281-1289 (1990).
9. Welch, R. A., et al. Extensive mosaic structure revealed by the complete genome sequence of uropathogenic Escherichia coli. P Natl Acad Sci USA 99, 17020-17024 (2002).
10. Garcia, E. C., Brumbaugh, A. R. & Mobley, H. L. T. Redundancy and specificity of Escherichia coli iron acquisition systems during urinary tract infection. Infect. Immun., IAI.01222-01210 (2011).
11. Carbonetti, N. H., et al. Aerobactin-mediated iron uptake by Escherichia coli isolates from human extraintestinal infections. Infect Immun 51, 966-968 (1986).
12. Johnson, J. R. Virulence factors in Escherichia coli urinary tract infection. Clinical microbiology reviews 4, 80-128 (1991).
13. Buchanan, S. K., et al. Crystal structure of the outer membrane active transporter FepA from Escherichia coli. Nature structural biology 6, 56-63 (1999).
14. Russo, T. A., et al. The Siderophore receptor IroN of extraintestinal pathogenic Escherichia coli is a potential vaccine candidate. Infect Immun 71, 7164-7169 (2003).
15. Alteri, C. J., Hagan, E. C., Sivick, K. E., Smith, S. N. & Mobley, H. L. Mucosal immunization with iron receptor antigens protects against urinary tract infection. PLoS Pathog 5, e1000586 (2009).
16. Ehmann, D. E., Shaw-Reid, C. A., Losey, H. C. & Walsh, C. T. The EntF and EntE adenylation domains of Escherichia coli enterobactin synthetase: Sequestration and selectivity in acyl-AMP transfers to thiolation domain cosubstrates. Proceedings of the National Academy of Sciences 97, 2509-2514 (2000).
17. Russo, T. A., et al. IroN Functions as a Siderophore Receptor and Is a Urovirulence Factor in an Extraintestinal Pathogenic Isolate of Escherichia coli. Infect Immun 70, 7156-7160 (2002).
18. Mori, K., et al. Endocytic delivery of lipocalin-siderophore-iron complex rescues the kidney from ischemia-reperfusion injury. J Clin Invest 115, 610-621 (2005).
19. Nickolas, T. L., et al. Sensitivity and specificity of a single emergency department measurement of urinary neutrophil gelatinase-associated lipocalin for diagnosing acute kidney injury. Ann Intern Med 148, 810-819 (2008).
20. Nickolas, T. L., et al. Diagnostic and prognostic stratification in the emergency department using urinary biomarkers of nephron damage: a multicenter prospective cohort study. J Am Coll Cardiol 59, 246-255 (2012).
21. Mishra, J., et al. Amelioration of ischemic acute renal injury by neutrophil gelatinase-associated lipocalin. J Am Soc Nephroi 15, 3073-3082 (2004).
22. Mishra, J., et al. Identification of neutrophil gelatinase-associated lipocalin as a novel early urinary biomarker for ischemic renal injury. J Am Soc Nephroi 14, 2534-2543 (2003).
23. Barasch, J. & Mori, K. Cell biology: iron thievery. Nature 432, 811-813 (2004).
24. Hung, C.-S., Dodson, K. W. & Hultgren, S. J. A murine model of urinary tract infection. Nat. Protocols 4, 1230-1243 (2009).
25. Hagberg, L., et al. Difference in susceptibility to gram-negative urinary tract infection between C3H/HeJ and C3H/HeN mice. Infect Immun 46, 839-844 (1984).
26. Chassin, C., et al. Renal collecting duct epithelial cells react to pyelonephritis-associated Escherichia coli by activating distinct TLR4-dependent and -independent inflammatory pathways. Journal of immunology (Baltimore, Md.: 1950) 177, 4773-4784 (2006).
27. van Adelsberg, J., Edwards, J. C., Takito, J., Kiss, B. & al-Awqati, Q. An induced extracellular matrix protein reverses the polarity of band 3 in intercalated epithelial cells. Cell 76, 1053-1061 (1994).
28. Xie, Y., et al. Identification of N-(quinolin-8-yl)benzenesulfonamides as agents capable of down-regulating NFkappaB activity within two separate high-throughput screens of NFkappaB activation. Bioorg Med Chem Lett 18, 329-335 (2008).
29. Flo, T. H., et al. Lipocalin 2 mediates an innate immune response to bacterial infection by sequestrating iron. Nature 432, 917-921 (2004).
30. Berger, T., et al. Lipocalin 2-deficient mice exhibit increased sensitivity to Escherichia coli infection but not to ischemia-reperfusion injury. P Natl Acad Sci USA 103, 1834-1839 (2006).
31. Poltorak, A., et al. Defective LPS signaling in C3H/HeJ and C57BL/10ScCr mice: mutations in Tlr4 gene. Science 282, 2085-2088 (1998).
32. Cowland, J. B., Sorensen, O. E., Sehested, M. & Borregaard, N. Neutrophil gelatinase-associated lipocalin is up-regulated in human epithelial cells by IL-1 beta, but not by TNF-alpha. Journal of immunology (Baltimore, Md.: 1950) 171, 6630-6639 (2003).
33. Kaye, D. Antibacterial Activity of Human Urine. Journal of Clinical Investigation 47, 2374-& (1968).
34. Asscher, A. W., Sussman, M., Waters, W. E., Davis, R. H. & Chick, S. Urine as a Medium for Bacterial Growth. Lancet 2, 1037-& (1966).
35. Carlsson, S., Wiklund, N. P., Engstrand, L., Weitzberg, E. & Lundberg, J. O. N. Effects of pH, nitrite, and ascorbic acid on nonenzymatic nitric oxide generation and bacterial growth in urine. Nitric Oxide-Biol Ch 5, 580-586 (2002).
36. Shohl, A. T. & Janney, J. H. The growth of Bacillus coli in urine at varying hydrogen ion concentrations. J Urology 1, 211-229 (1917).
37. Schwartz, G. J., Barasch, J. & Alawqati, Q. Plasticity of Functional Epithelial Polarity. Nature 318, 368-371 (1985).
38. Semsey, S., et al. Genetic regulation of fluxes: iron homeostasis of Escherichia coli. Nucleic Acids Res 34, 4960-4967 (2006).
39. Snyder, J. A., et al. Transcriptome of uropathogenic Escherichia coli during urinary tract infection. Infect Immun 72, 6373-6381 (2004).
40. Hagan, E. C., Lloyd, A. L., Rasko, D. A., Faerber, G. J. & Mobley, H. L. Escherichia coli global gene expression in urine from women with urinary tract infection. PLoS Pathog 6, e1001187 (2010).
41. Zasloff, M. Antimicrobial peptides, innate immunity, and the normally sterile urinary tract. J Am Soc Nephrol 18, 2810-2816 (2007).
42. Dooley, T. P., Miranda, M., Jones, N. C. & DePamphilis, M. L. Transactivation of the adenovirus EIIa promoter in the absence of adenovirus E1A protein is restricted to mouse oocytes and preimplantation embryos. Development (Cambridge, England) 107, 945-956 (1989).
43. Klose, A. D. & Larsen, E. W. Light transport in biological tissue based on the simplified spherical harmonics equations. J Comput Phys 220, 441-470 (2006).
44. Zhang, Z., et al. Improved techniques for kidney transplantation in mice. Microsurgery 16, 103-109 (1995).
45. Li, J. Y., et al. Scara5 is a ferritin receptor mediating non-transferrin iron delivery. Developmental cell 16, 35-46 (2009).
46. Zhu, X. D. & Sadowski, P. D. Cleavage-dependent ligation by the FLP recombinase. Characterization of a mutant FLP protein with an alteration in a catalytic amino acid. The Journal of biological chemistry 270, 23044-23054 (1995).
47. Sauer, B. Functional expression of the cre-lox site-specific recombination system in the yeast Saccharomyces cerevisiae. Molecular and cellular biology 7, 2087-2096 (1987).
48. Poltorak A, He X, Smirnova I, Liu M Y, Van Huffel C, Du X, Birdwell D, Alejos E, Silva M, Galanos C, Freudenberg M, Ricciardi-Castagnoli P, Layton B, Beutler B.), Defective LPS signaling in C3H/HeJ and C57BL/10ScCr mice: mutations in Tlr4 gene., Science. 1998 Dec. 11; 282(5396):2085-8.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

What is claimed is:

1. A method for treating or preventing a bacterial infection of the urinary tract, or urosepsis associated therewith, in a subject, the method comprising administering to the subject a therapeutically effective amount of one or more agents selected from the group consisting of: (a) an agent that stimulates genito-urinary tract epithelial cells to produce NGAL, (b) an NGAL protein, and (c) a functional derivative of an NGAL protein.

2. The method of claim 1, wherein the subject is a human.

3. The method of claim 1, wherein the bacterial infection is an infection with a catecholate siderophore-dependent bacterium.

4. The method of claim 1, wherein the bacterial infection is an infection with an enterochelin-dependent bacterium.

5. The method of claim 1, wherein the bacterial infection is an infection with an enterochelin-dependent E. coli bacterium.

6. The method of claim 1, further comprising administering to the subject an additional bacteriostatic or antibiotic agent.

7. The method of claim 1, wherein the genito-urinary tract epithelial cells are kidney epithelial cells.

8. The method of claim 7, wherein the kidney epithelial cells are collecting duct epithelial cells or epithelial cells of the thick ascending limb of Henlé.

9. The method of claim 1, wherein the agent that stimulates genito-urinary tract epithelial cells to produce NGAL is an NFκB activator, a NRF2 modulator, or a HIF modulator.

10. The method of claim 1, wherein the agent that stimulates genito-urinary tract epithelial cells to produce NGAL is an activator of a TLR-NFκB pathway.

11. The method of claim 10, wherein the activator of a TLR-NFκB pathway is an activator of a TLR4-NFκB pathway.

12. The method of claim 10, wherein the activator of a TLR-NFκB pathway is an activator of a TLR11-NFκB pathway.

13. The method of claim 1, wherein the agent that stimulates genito-urinary tract epithelial cells to produce NGAL is a non-toxic derivative of either lipid A, lipopolysaccharide, or endotoxin.

14. The method of claim 1, wherein the agent that stimulates genito-urinary tract epithelial cells to produce NGAL, and/or the NGAL or functional derivative thereof, are administered systemically.

15. The method of claim 1, wherein the agent that stimulates genito-urinary tract epithelial cells to produce NGAL, and/or the NGAL or functional derivative thereof, are administered locally to the genitourinary tract.

16. A method for treating or preventing bacterial infection of the urinary tract, or urosepsis associated therewith, in a subject, the method comprising stimulating genito-urinary tract epithelial cells of the subject to produce NGAL.

17. The method of claim 16, wherein the subject is a human.

18. The method of claim 16, wherein the bacterial infection is an infection with a catecholate siderophore-dependent bacterium.

19. The method of claim 16, wherein the bacterial infection is an infection with an enterochelin-dependent bacterium.

20. The method of claim 16, wherein the bacterial infection is an infection with an enterochelin-dependent E. coli bacterium.

21. The method of claim 16, wherein the genito-urinary tract epithelial cells are kidney epithelial cells.

22. The method of claim 21, wherein the kidney epithelial cells are collecting duct epithelial cells or epithelial cells of the thick ascending limb of Henlé.

23. The method of claim 16, wherein the step of stimulating genito-urinary tract epithelial cells to produce NGAL comprises administering to the subject a therapeutically effective amount one or more agent selected from the group consisting of: (a) a therapeutically effective derivative of lipid A, (b) a therapeutically effective derivative of lipopolysaccharide, (c) a therapeutically effective derivative of endotoxin, (d) an activator of the TLR4-NFkB pathway, (e) an activator of the TLR11-NFkB pathway, (f) an NFκB activator, (g) a NRF2 modulator, and (h) a HIF modulator.

24. The method of claim 23, wherein the agent is administered systemically.

25. The method of claim 23, wherein the agent is administered locally to the genitourinary tract.

26. A pharmaceutical composition for use in treating or preventing a bacterial urinary tract infection, or urosepsis associated therewith, the composition comprising a therapeutically effective amount of an agent that stimulates genito-urinary tract epithelial cells to produce NGAL, and a therapeutically effective amount of an NGAL protein or a functional derivative thereof.

27. The composition of claim 26, wherein the agent that stimulates genito-urinary tract epithelial cells to produce NGAL is selected from the group consisting of: (a) a non-toxic derivative of lipid A, (b) a non-toxic derivative of lipopolysaccharide, (c) a non-toxic derivative of endotoxin, (d) an activator of the TLR4-NFkB pathway, (e) an activator of the TLR11-NFkB pathway, (f) an NFκB activator, (g) a NRF2 modulator, and (h) a HIF modulator.

28. A method of identifying an agent that stimulates epithelial cells of the urinary tract to produce NGAL mRNA or NGAL protein, the method comprising:
(a) providing a test population of urinary tract epithelial cells and a control population of urinary tract epithelial cells,
(b) contacting the test population of urinary tract epithelial cells with one or more test agents,
(c) contacting the control population of urinary tract epithelial cells with no agent or with one or more negative control agents, and
(d) determining the level of NGAL mRNA or NGAL protein in the test population and the control population, or in a culture supernatant thereof,
wherein a level of NGAL mRNA or NGAL protein in the test population, or a culture supernatant thereof, that is higher than the level of NGAL mRNA or NGAL protein in the control population, or a culture supernatant thereof, indicates that the test agent is an agent that stimulates production of NGAL mRNA or NGAL protein by the urinary tract epithelial cells.

29. A method of identifying an agent that stimulates epithelial cells of the urinary tract to produce NGAL mRNA or NGAL protein, the method comprising:
(a) providing a population of urinary tract epithelial cells,
(b) determining the control level of NGAL mRNA or NGAL protein in the population of urinary tract epithelial cells, or in a culture supernatant thereof, wherein the control level is the level of NGAL mRNA or NGAL protein present prior to contacting the urinary tract epithelial cells with one or more test agents,
(c) contacting the urinary tract epithelial cells with one or more test agents,
(d) determining the test level of NGAL mRNA or NGAL protein in the population of urinary tract epithelial cells, or in a culture supernatant thereof, wherein the test level is the level of NGAL mRNA or NGAL protein present subsequent to contacting the urinary tract epithelial cells with the one or more test agents,
wherein if the test level of NGAL mRNA or NGAL protein exceeds the control level of NGAL mRNA or NGAL protein, the test agent is an agent that stimulates production of NGAL mRNA or NGAL protein by the urinary tract epithelial cells.


SEQUENCE LISTING (49 sequences)

SEQ ID NO: 1 - DNA, 20 bp, Artificial Sequence
  Description of Artificial Sequence Synthetic primer
ctcagaacttgatccctgcc

SEQ ID NO: 2 - DNA, 20 bp, Artificial Sequence
  Description of Artificial Sequence Synthetic primer
tccttgaggcccagacactt

SEQ ID NO: 3 - DNA, 20 bp, Artificial Sequence
  Description of Artificial Sequence Synthetic primer
ctaaggccaaccgtgaaaag

SEQ ID NO: 4 - DNA, 20 bp, Artificial Sequence
  Description of Artificial Sequence Synthetic primer
tctcagctgtggtggtgaag

SEQ ID NO: 5 - DNA, 24 bp, Artificial Sequence
  Description of Artificial Sequence Synthetic primer
aagactcaactcagaacttgatcc

SEQ ID NO: 6 - DNA, 24 bp, Artificial Sequence
  Description of Artificial Sequence Synthetic primer
gaggaagcttggacaggaatctgg

SEQ ID NO: 7 - DNA, 24 bp, Artificial Sequence
  Description of Artificial Sequence Synthetic primer
acgacaacatcatcttctctgtcc

SEQ ID NO: 8 - DNA, 27 bp, Artificial Sequence
  Description of Artificial Sequence Synthetic primer
tgcgaggccagaggccacttgtgtagc

SEQ ID NO: 9 - DNA, 27 bp, Artificial Sequence
  Description of Artificial Sequence Synthetic primer
gtccttctcactttgacagaagtcagg

SEQ ID NO: 10 - DNA, 27 bp, Artificial Sequence
  Description of Artificial Sequence Synthetic primer
cacatctcatgctgctcagatagccac

SEQ ID NO: 11 - DNA, 20 bp, Artificial Sequence
  Description of Artificial Sequence Synthetic primer
ctcagaacttgatccctgcc

SEQ ID NO: 12 - DNA, 20 bp, Artificial Sequence
  Description of Artificial Sequence Synthetic primer
tccttgaggcccagagactt

SEQ ID NO: 13 - DNA, 20 bp, Artificial Sequence
  Description of Artificial Sequence Synthetic primer
ctaaggccaaccgtgaaaag

SEQ ID NO: 14 - DNA, 20 bp, Artificial Sequence
  Description of Artificial Sequence Synthetic primer
tctcagctgtggtggtgaag

SEQ ID NO: 15 - DNA, 22 bp, Artificial Sequence
  Description of Artificial Sequence Synthetic primer
aagttggtgttgacgttgtcgc

SEQ ID NO: 16 - DNA, 20 bp, Artificial Sequence
  Description of Artificial Sequence Synthetic primer
agcgcctttaacgaacatcg

SEQ ID NO: 17 - DNA, 20 bp, Artificial Sequence
  Description of Artificial Sequence Synthetic primer
agcgtgttgagattgttcgc

SEQ ID NO: 18 - DNA, 24 bp, Artificial Sequence
  Description of Artificial Sequence Synthetic primer
aaaccactgctttgtccttcctgc

SEQ ID NO: 19 - DNA, 25 bp, Artificial Sequence
  Description of Artificial Sequence Synthetic primer
ctgtttctcaatcaatgggccagtg

SEQ ID NO: 20 - DNA, 25 bp, Artificial Sequence
  Description of Artificial Sequence Synthetic primer
tatgccaccgacaataccgatatgg

SEQ ID NO: 21 - DNA, 25 bp, Artificial Sequence
  Description of Artificial Sequence Synthetic primer
agctggacttttagcgtaacggctg

SEQ ID NO: 22 - DNA, 25 bp, Artificial Sequence
  Description of Artificial Sequence Synthetic primer
cgacatcttatccaccagaaaccgc

SEQ ID NO: 23 - DNA, 22 bp, Artificial Sequence
  Description of Artificial Sequence Synthetic primer
gccactggcccctgattgtgaa

SEQ ID NO: 24 - DNA, 21 bp, Artificial Sequence
  Description of Artificial Sequence Synthetic primer
aatccgcaagaaaatggcccg

SEQ ID NO: 25 - DNA, 24 bp, Artificial Sequence
  Description of Artificial Sequence Synthetic primer
tagctgaaacggagcgactggacg

SEQ ID NO: 26 - DNA, 22 bp, Artificial Sequence
  Description of Artificial Sequence Synthetic primer
acgtcggcggtgcgtttaacct

SEQ ID NO: 27 - DNA, 20 bp, Artificial Sequence
  Description of Artificial Sequence Synthetic primer
ccaaagcgcagggcatcaaa

SEQ ID NO: 28 - DNA, 21 bp, Artificial Sequence
  Description of Artificial Sequence Synthetic primer
aaaacaagcttccgcacgccg

SEQ ID NO: 29 - DNA, 23 bp, Artificial Sequence
  Description of Artificial Sequence Synthetic primer
agcggggatgatttcctcaacac

SEQ ID NO: 30 - DNA, 23 bp, Artificial Sequence
  Description of Artificial Sequence Synthetic primer
gctgaggattttactgccacgcc

SEQ ID NO: 31 - DNA, 22 bp, Artificial Sequence
  Description of Artificial Sequence Synthetic primer
cgctgttcggtccggtactcaa

SEQ ID NO: 32 - DNA, 22 bp, Artificial Sequence
  Description of Artificial Sequence Synthetic primer
tgaactggccctgttcccggat

SEQ ID NO: 33 - DNA, 20 bp, Artificial Sequence
  Description of Artificial Sequence Synthetic primer
acggccaaagccagaataac

SEQ ID NO: 34 - DNA, 22 bp, Artificial Sequence
  Description of Artificial Sequence Synthetic primer
ttaccgtaaagcacggaaaccg

SEQ ID NO: 35 - DNA, 25 bp, Artificial Sequence
  Description of Artificial Sequence Synthetic primer
ccggtctggattccgaagctggtta

SEQ ID NO: 36 - DNA, 23 bp, Artificial Sequence
  Description of Artificial Sequence Synthetic primer
agaccatctggtggagtttgccg

SEQ ID NO: 37 - DNA, 22 bp, Artificial Sequence
  Description of Artificial Sequence Synthetic primer
attaccaaacgtcccaccaacg

SEQ ID NO: 38 - DNA, 22 bp, Artificial Sequence
  Description of Artificial Sequence Synthetic primer
aaacgcgtggtaagagcatcac

SEQ ID NO: 39 - DNA, 24 bp, Artificial Sequence
  Description of Artificial Sequence Synthetic primer
cccatacgcaacaggcaattgatg

SEQ ID NO: 40 - DNA, 23 bp, Artificial Sequence
  Description of Artificial Sequence Synthetic primer
caccctgcgcctaagtctcatga

SEQ ID NO: 41 - DNA, 24 bp, Artificial Sequence
  Description of Artificial Sequence Synthetic primer
cttcagcgaagaaatggcagacca

SEQ ID NO: 42 - DNA, 25 bp, Artificial Sequence
  Description of Artificial Sequence Synthetic primer
tgatataccggtcgtgatgcaaacc

SEQ ID NO: 43 - DNA, 25 bp, Artificial Sequence
  Description of Artificial Sequence Synthetic primer
ggcgttacagaaaaacctggttgcc

SEQ ID NO: 44 - DNA, 24 bp, Artificial Sequence
  Description of Artificial Sequence Synthetic primer
atgggtatgttgcgtatcgccaaa

SEQ ID NO: 45 - DNA, 22 bp, Artificial Sequence
  Description of Artificial Sequence Synthetic primer
gcactcacctgctcgatcgcat

SEQ ID NO: 46 - DNA, 25 bp, Artificial Sequence
  Description of Artificial Sequence Synthetic primer
tctcatccataaccaagcctggtgc

SEQ ID NO: 47 - DNA, 65 bp, Artificial Sequence
  Description of Artificial Sequence Synthetic oligonucleotide
tcctgcccatctctgctcactgtccccctgcagccagacttccggagcgatcaggtaggaccctg

SEQ ID NO: 48 - DNA, 59 bp, Artificial Sequence
  Description of Artificial Sequence Synthetic oligonucleotide
tctagacgcggtggtaccataacttgctatagcatacattatacgaagttatggtacct

SEQ ID NO: 49 - DNA, 60 bp, Artificial Sequence
  Description of Artificial Sequence Synthetic oligonucleotide
cgatctcagtcaggacttgccctgatgaggagtccagattcctgtccaagcttcctcatt